SEQUENCE LISTING (15 sequences)

SEQ ID NO: 1 - protein, 15 aa, synthetic construct
  REGION 1..15: note = Synthetic
KASQSVDYDGDSYMN

SEQ ID NO: 2 - protein, 7 aa, synthetic construct
  REGION 1..7: note = Synthetic
DASNLES

SEQ ID NO: 3 - protein, 9 aa, synthetic construct
  REGION 1..9: note = Synthetic
QQSNEDPWT

SEQ ID NO: 4 - protein, 5 aa, synthetic construct
  REGION 1..5: note = Synthetic
DDYIH

SEQ ID NO: 5 - protein, 17 aa, synthetic construct
  REGION 1..17: note = Synthetic
RIDPADGHTKYAPKFQV

SEQ ID NO: 6 - protein, 10 aa, synthetic construct
  REGION 1..10: note = Synthetic
YGYGREVFDY

SEQ ID NO: 7 - protein, 111 aa, synthetic construct
  REGION 1..111: note = Synthetic
DIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIK

SEQ ID NO: 8 - protein, 119 aa, synthetic construct
  REGION 1..119: note = Synthetic
QVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSS

SEQ ID NO: 9 - protein, 218 aa, synthetic construct
  REGION 1..218: note = Synthetic
DIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 446 aa, synthetic construct
  REGION 1..446: note = Synthetic
QVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 11 - protein, 327 aa, synthetic construct
  REGION 1..327: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 327 aa, synthetic construct
  REGION 1..327: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 327 aa, synthetic construct
  REGION 1..327: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 107 aa, synthetic construct [sequence omitted]

SEQ ID NO: 15 - protein, 673 aa, Homo sapiens
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

(19) United States
(12) Patent Application Publication
(10) Pub. No.: US 2024/0117021 A1
(43) Pub. Date: Apr. 11, 2024

(54) ANTI-COMPLEMENT C1S ANTIBODY FORMULATION

(71) Applicant: Bioverativ USA Inc., Waltham, MA (US)

(72) Inventors: Sanket Patke, Burlington, MA (US); Anvay Ashish Ukidve, Cambridge, MA (US)

(73) Assignee: Bioverativ USA Inc., Waltham, MA (US)

(21) Appl. No.: 18/334,567

(22) Filed: Jun. 14, 2023

Related U.S. Application Data
(60) Provisional application No. 63/352,475, filed on Jun. 15, 2022.

(51) Int. Cl. C07K 16/18; A61K 47/12; A61K 47/18; A61K 47/26

(52) U.S. Cl. CPC C07K 16/18; A61K 47/12; A61K 47/183; A61K 47/26; C07K 2317/565

(57) ABSTRACT

Provided herein are compositions comprising a humanized antibody that specifically binds complement component C1s (anti-C1s antibody) that are capable of stable long-term storage. The compositions may contain, in addition to the humanized antibody, arginine or a salt thereof.

### RELATED APPLICATION

This application claims the benefit under 35 U.S.C. § 119(e) of U.S. provisional application Ser. No. 63/352,475, filed Jun. 15, 2022, the entire content of which is incorporated herein by reference.

### REFERENCE TO AN ELECTRONIC SEQUENCE LISTING

The contents of the electronic sequence listing (B155370017WO00-SEQ-JRV.xml; Size: 21,726 bytes; and Date of Creation: Jun. 9, 2023) is herein incorporated by reference in its entirety.

### BACKGROUND

The complement system is a well-known effector mechanism of the immune response, providing not only protection against pathogens and other harmful agents but also recovery from injury. The classical complement pathway is triggered by activation of the first component of complement, referred to as the C1 complex, which includes C1q, C1r, and C1s proteins. Upon binding of C1 to an immune complex, the C1s component, a di-isopropyl fluorophosphate (DFP)-sensitive serine protease, cleaves complement components C4 and C2 to initiate activation of the classical complement pathway. The classical complement pathway appears to play a role in many diseases and disorders and there is a need for stable formulations of antibodies targeting this pathway.

### SUMMARY

The present disclosure provides compositions comprising a humanized antibody that specifically binds complement C1s protein (i.e., a humanized anti-complement C1s antibody, also referred to herein as a “humanized anti-C1s antibody,” a “humanized C1s antibody,” or a “subject antibody”). The present disclosure also provides methods of treating a complement-mediated disease or disorder, comprising administering a composition of the present disclosure.

The subject antibody was found to have a higher propensity to self-associate and to also have a tendency for isomerization at an aspartic acid residue at position number 32 in the light chain variable domain sequence, located in the first complementarity determining region (CDR1) of the light chain (LC D32 isomerization). A set of comprehensive studies was performed to understand the impact of formulation composition and process conditions on the biophysical and chemical stability of the subject antibody. The studies described herein demonstrate that the presence of arginine or a salt thereof reduces the rate of D32 isomerization. Resistance to isomerization was observed in sample formulations using different batches of subject antibody under refrigerated, ambient and accelerated storage conditions when using the compositions of the disclosure. The compositions of the disclosure preserve antibody stability during long term storage and provide protection against physicochemical stresses, while also inhibiting LC D32 isomerization, antibody aggregation, and deamidation. The compositions of the disclosure further allow the subject antibody to be formulated as a liquid.

In one aspect, the disclosure provides a composition comprising: (a) a humanized antibody that specifically binds complement component C1s, wherein the antibody comprises a light chain (LC) complementarity determining region (CDR) 1 comprising the amino acid sequence of SEQ ID NO: 1, a LC CDR2 comprising the amino acid sequence of SEQ ID NO: 2, a LC CDR3 comprising the amino acid sequence of SEQ ID NO: 3 and a heavy chain (HC) CDR1 comprising the amino acid sequence of SEQ ID NO: 4, an HC CDR2 comprising the amino acid sequence of SEQ ID NO: 5, and an HC CDR3 comprising the amino acid sequence of SEQ ID NO: 6; and (b) about 50 mM to about 200 mM arginine or a salt thereof.

In some embodiments, the composition comprises about 100 mM to about 200 mM or 20 about 100 mM to about 150 mM of arginine or a salt thereof. In some embodiments, the arginine salt is arginine hydrochloride, arginine citrate, arginine oxalate, arginine phosphate, arginine succinate, or arginine tartrate. In some embodiments, the arginine salt is arginine hydrochloride. In some embodiments, the composition comprises about 150 mM arginine hydrochloride. In some embodiments, the arginine hydrocholoride is L-arginine hydrochloride.

In some embodiments, the composition comprises a buffer. In some embodiments, the composition comprises a buffer at a concentration of about 1 mM to about 50 mM, about 5 mM to about 25 mM, or about 10 mM to about 20 mM. In some embodiments, the buffer is histidine, acetate, citrate, oxalate, phosphate, succinate, or tartrate. In some embodiments, the buffer is histidine. In some embodiments, the composition comprises about 10 mM histidine.

In some embodiments, the composition further comprises a stabilizer. In some embodiments, the composition further comprises a stabilizer at a concentration of about 1% to about 8% (w/v) or about 1% to about 5% (w/v). In some embodiments, the stabilizer is sucrose, sorbitol, or trehalose. In some embodiments, the stabilizer is sucrose. In some embodiments, the composition comprises about 3% (w/v) sucrose.

In some embodiments, the composition further comprises a chelator. In some embodiments, the composition comprises a chelator at a concentration of about 1 μM to about 50 μM, about 5 μM to about 25 μM or about 10 μM to about 25 μM. In some embodiments, the chelator is diethylenetriamine pentaacetate (DTPA), ethylenediaminetetraacetic acid (EDTA). In some embodiments, the composition comprises about 10 μM diethylenetriamine pentaacetate (DTPA). In some embodiments, the chelator is methionine. In some embodiments, the composition comprises about 5 mM to about 10 mM methionine.

In some embodiments, the composition further comprises a surfactant. In some embodiments, the composition comprises a surfactant at a concentration of about 0.01% to about 0.1% (w/v) or about 0.03% to about 0.06% (w/v). In some embodiments, the surfactant is polysorbate 80 (PS80) or poloxamer 188 (P188). In some embodiments, the surfactant is PS80, optionally wherein the composition comprises about 0.06% (w/v) polysorbate 80.

In some embodiments, the composition has a pH of about 6 to about 7.5, about 6 to about 7, about 6.5 to about 7.5, or about 6.5 to about 7.1. In some embodiments, the composition has a pH of about 6.8.

In some embodiments, the composition comprises about 50 mg/mL to about 250 mg/mL, or about 100 mg/mL to about 200 mg/mL of the anti-C1s antibody. In some embodiments, the composition comprises about 150 mg/mL of the anti-C1s antibody.

In some embodiments, the composition comprises: (a) about 50 mg/mL to about 250 mg/mL of the antibody; (b) about 50 mM to about 200 mM arginine HCl; (c) about 1 mM to about 50 mM histidine; (d) about 1% to about 8% (w/v) sucrose; (e) about 1 μM to about 50 μM diethylenetriamine pentaacetate (DTPA); and (f) about 0.01% to about 0.1% (w/v) polysorbate 80 (PS80), and the composition has a pH of about 6 to about 7.5. In some embodiments, the arginine hydrocholoride is L-arginine hydrochloride.

In some embodiments, the composition comprises: (a) about 150 mg/mL of the antibody; (b) about 150 mM arginine HCl; (c) about 10 mM histidine; (d) about 3% (w/v) sucrose; (e) about 10 μM DTPA; and (f) about 0.06% (w/v) PS80, and the composition has a pH of about 6.5 to about 7.1, or about 6.8. In some embodiments, the arginine hydrocholoride is L-arginine hydrochloride.

In some embodiments, the antibody comprises a light chain variable region (VL) comprising the amino acid sequence of SEQ ID NO: 7 and a heavy chain variable region (VH) comprising the amino acid sequence of SEQ ID NO: 8. In some embodiments, the antibody is a Fab fragment, a F(ab′)2 fragment, a scFv, or a Fv. In some embodiments, the antibody comprises a heavy chain constant region of the isotype IgG4. In some embodiments, the IgG4 constant region comprises a proline, a glutamic acid, a leucine, and a serine substitutions at amino acid residues 108, 115, 308, and 314, respectively, relative to the IgG4 constant region sequence of SEQ ID NO: 11. In some embodiments, the antibody comprises a heavy chain constant region comprising the amino acid sequence of SEQ ID NO: 13. In some embodiments, the antibody comprises a light chain comprising the amino acid sequence of SEQ ID NO: 9 and a heavy chain comprising the amino acid sequence of SEQ ID NO: 10.

In some embodiments, the antibody has a lower isomerization rate at D32 of the light chain, located in CDR1, as compared to the antibody in a corresponding formulation without arginine or a salt thereof, optionally wherein the D32 isomerization is determined by total peptide map analysis. In some embodiments, the antibody (i) has an isomerization rate that is at least about 2-3% lower per week at 40° C., at least about 1-3% lower per month at 25° C. or at least about 0.4-0.6% lower per month at 5° C., as compared to the antibody in a corresponding formulation without arginine or a salt thereof; or (ii) has an isomerization rate after 12 weeks of storage at 25° C. that is about 10% lower as compared to the antibody in a corresponding formulation without arginine or a salt thereof.

In some embodiments, the antibody has an isomerization rate of less than 7.5% after 12 weeks of storage at 5° C., less than 20% after 12 weeks of storage at 25° C., or less than 35% after 12 weeks of storage at 40° C.

In some embodiments, the composition is in liquid, lyophilized, or reconstituted lyophilized form. In some embodiments, the composition is in liquid form.

In one aspect, the present disclosure provides a container containing a composition of the disclosure. In some embodiments, the container is a vial or a syringe. In some embodiments, the container is a syringe. In some embodiments, the syringe is a pre-filled syringe.

In one aspect, the present disclosure provides a kit or an article of manufacture, comprising a container of the disclosure.

In one aspect, the present disclosure provides a pharmaceutical unit dosage form suitable for parenteral administration to a human, comprising a composition of the disclosure in a container.

In one aspect, the present disclosure provides a method comprising administering to a human a composition of the disclosure.

In one aspect, the present disclosure provides a method of reducing the level of a complement component cleavage product in a human, the method comprising administering to the human a composition of the disclosure.

In one aspect, the present disclosure provides a method of inhibiting C1s-mediated cleavage of a complement component in a human, the method comprising administering to the human a composition of the disclosure.

In some embodiments, the human has cold agglutinin disease (CAD), immunothrombocytopenic purpura (ITP), chronic inflammatory demyelinating polyradiculoneuropathy (CIDP), or antibody mediated rejection (AMR).

In one aspect, the present disclosure provides a method of treating a complement-mediated disease in a human in need thereof, comprising administering to the human a composition of the disclosure. In some embodiments, the complement-mediated disease is cold agglutinin disease (CAD), immunothrombocytopenic purpura (ITP), chronic inflammatory demyelinating polyradiculoneuropathy (CIDP), or antibody mediated rejection (AMR).

In some embodiments, the administration is intravenous or subcutaneous.

In one aspect, the present disclosure provides a drug delivery device comprising a primary container containing a composition of the disclosure, wherein the drug delivery device is a sleeve-triggered auto-injector with manual needle insertion.

Each of International Publication Nos. WO 2014/066744, filed Oct. 25, 2013, entitled Anti-Complement C1s Antibodies and Uses Thereof, WO 2016/164358, filed Apr. 5, 2016, entitled Humanized Anti-C1s Antibodies and Methods of Use Thereof, and WO 2018/071676, filed Oct. 12, 2017, entitled Anti-C1s Antibodies and Methods of Use Thereof, is herein incorporated by reference in its entirety.

### BRIEF DESCRIPTION OF THE DRAWINGS

FIG. 1 shows the Biacore binding versus LC D32 isomerization rate or forced isomerization study.

FIGS. 2A-2D show the effect of pH and stabilizer on LC D32 isomerization. FIG. 2A shows the effect of pH and stabilizer at 5° C. FIG. 2B shows the effect of pH and stabilizer at 25° C. FIG. 2C shows the effect of pH and stabilizer at 40° C. FIG. 2D summarizes the effect of pH and stabilizer on the isomerization rate at the different temperatures.

FIGS. 3A-3E show the analytics for orbital shaking study. FIG. 3A: HMW species, FIG. 3B: Turbidity, FIGS. 3C-3E: Sub-visible particles (≤2 μm, ≤10 μm, ≤25 μm particles/mL). (h=hours; d=days).

FIGS. 4A-4E show the analytics for wrist action shaking study. FIG. 4A: HMW species, FIG. 4B: Turbidity, FIGS. 4C-4E: Sub-visible particles (≤2 μm, ≤10 μm, ≤25 μm particles/mL). (h=hours).

FIGS. 5A-5D show the analytics for wrist action shaking+silicone spiking study. FIG. 5A: Turbidity, FIGS. 5B-5D: Sub-visible particles (≤2 μm, ≤10 μm, ≤25 μm particles/mL). (h=hours).

FIGS. 6A-6F show the analytics for IV Bag dilution study. FIG. 6A: HMW species, FIG. 6B: Turbidity, FIG. 6C: Concentration, FIGS. 6D-6F: Sub-visible particles (≤2 μm, ≤10 μm, ≤25 μm particles/mL). (h=hours).

FIGS. 7A-7F show the analytics for photostability study. FIG. 7A: HMW species, FIG. 7B: Turbidity, FIG. 7C: PS80 concentration, FIGS. 7D-7F: Sub-visible particles (≤2 μm, ≤10 μm, ≤25 μm particles/mL). (h=hours; d=days).

FIGS. 8A-8E show the analytics from Batch 1 chelator studies. FIG. 8A shows the PS80 concentration for 10 μM EDTA vs. 10 μM DTPA, FIG. 8B shows the % PS80 remaining for 10/50 μM EDTA vs. 10/50 μM DTPA, FIG. 8C: Ester profile, FIG. 8D: HMW species, FIG. 8E: Biacore analysis. (W=weeks; M=months; C=degrees Celsius; e.g., “3M-25C” denotes 3 months of storage at 25° C.).

FIG. 9 shows the visual analysis of Batch 1 formulations stored at 40° C. for 3 months.

FIGS. 10A-10E show the analytics for Batch 2 chelator study. FIG. 10A shows the PS80 concentration for 10 μM EDTA vs 10 μM DTPA, FIG. 10B shows the % PS80 remaining for 10/50 μM EDTA vs. 10/50 μM DTPA, FIG. 10C: HMW species, FIG. 10D: LMW species, FIG. 10E: Biacore analysis. (W=weeks; M=months; C=degrees Celsius; e.g., “3M-25C” denotes 3 months of storage at 25° C.).

FIGS. 11A-11E show the analytics for Batch 3 chelator study. FIG. 11A shows the PS80 concentration for 10 μM EDTA vs 10 μM DTPA, FIG. 11B shows the % PS80 remaining for 10/50 μM EDTA vs. 10/50 μM DTPA, FIG. 11C: HMW species, FIG. 11D: LMW species, FIG. 11E: Biacore analysis. (W=weeks; M=months; C=degrees Celsius; e.g., “3M-25C” denotes 3 months of storage at 25° C.).

FIGS. 12A-12E show the analytics for Batch 4 chelator study. FIG. 12A shows the PS80 concentration for 10 μM EDTA vs 10 μM DTPA, FIG. 12B shows the % PS80 comparison-10/50 μM EDTA vs. 10/50 μM DTPA, FIG. 12C: HMW species, FIG. 12D: LMW species, FIG. 12E: Biacore analysis. (W=weeks; M=months; C=degrees Celsius; e.g., “3M-25C” denotes 3 months of storage at 25° C.).

FIGS. 13A-13E show the analytics from pre-filled syringe (PFS) stability study of buffer type (H=histidine buffer; K=potassium phosphate buffer) and pH (value noted after buffer type) for Batch 1 samples. FIG. 13A: HMW species, FIG. 13B: Concentration, FIG. 13C: pH, FIG. 13D: Turbidity, FIG. 13E: PS80 concentration. (W=weeks; M=months; C=degrees Celsius; e.g., “3M-25C” denotes 3 months of storage at 25° C.).

FIGS. 14A-14C show the sub-visible particle analysis for PFS stability study of buffer type (H=histidine buffer; K=potassium phosphate buffer) and pH (value noted after buffer type) for Batch 1 samples. FIG. 14A: Sub-visible particles≤2 μm particles/mL, FIG. 14B: Sub-visible particles≤10 μm particles/mL, FIG. 14C: Sub-visible particles≤25 μm particles/mL. (W=weeks; M=months; C=degrees Celsius; e.g., “3M-25C” denotes 3 months of storage at 25° C.) (OMPI=OMPI PFS; BD=BD PFS).

FIGS. 15A-15E show the analytics from vial stability study of buffer type (H=histidine buffer; K=potassium phosphate buffer) and pH (value noted after buffer type) for Batch 1 samples. FIG. 15A: HMW species, FIG. 15B: Concentration, FIG. 15C: pH, FIG. 15D: Turbidity, FIG. 15E: PS80 concentration. (W=weeks; M=months; C=degrees Celsius; e.g., “3M-25C” denotes 3 months of storage at 25° C.).

FIGS. 16A-16C show the sub-visible particle analysis for vial stability study of buffer type (H=histidine buffer; K=potassium phosphate buffer) and pH (value noted after buffer type) for Batch 1 samples. FIG. 16A: Sub-visible particles≤2 μm particles/mL, FIG. 16B: Sub-visible particles≤10 μm particles/mL, FIG. 16C: Sub-visible particles≤25 μm particles/mL. (W=weeks; M=months; C=degrees Celsius; e.g., “3M-25C” denotes 3 months of storage at 25° C.).

FIGS. 17A-17G show the analytics for the frozen-storage stability study for Batch 1 samples. FIG. 17A: HMW species, FIG. 17B: Concentration, FIG. 17C: pH, FIG. 17D: Turbidity, FIGS. 17E-17G: Sub-visible particles (≤2 μm, ≤10 μm, ≤25 μm particles/mL). (M=months).

FIGS. 18A-18E show the peptide map and Biacore binding analysis for Batch 1 samples. FIG. 18A: LC D32 Isomerization, FIG. 18B: heavy chain (HC) M251 Oxidation, FIG. 18C: HC PENNYK Deamidation, FIG. 18D: HC VSNK Deamidation, FIG. 18E: Biacore analysis. (W=weeks; M=months; C=degrees Celsius; e.g., “6M-25C” denotes 6 months of storage at 25° C.).

FIGS. 19A-19G show the analytics from the PFS stability study for Batch 2 samples. FIG. 19A: HMW species, FIG. 19B: LMW species, FIG. 19C: Concentration, FIG. 19D: pH, FIG. 19E: Turbidity, FIG. 19F: PS80 concentration, FIG. 19G: Charged variant analysis. (W=weeks; M=months; C=degrees Celsius; e.g., “3M-25C” denotes 3 months of storage at 25° C.).

FIGS. 20A-20C show the sub-visible particle analysis for PFS stability study for Batch 2 samples. FIG. 20A: Sub-visible particles≤2 μm particles/mL, FIG. 20B: Sub-visible particles≤10 μm particles/mL, FIG. 20C: Sub-visible particles≤25 μm particles/mL. (W=weeks; M=months; C=degrees Celsius; e.g., “3M-25C” denotes 3 months of storage at 25° C.).

FIGS. 21A-21G show the analytics from vial stability study for Batch 2 samples. FIG. 21A: HMW species, FIG. 21B: LMW species, FIG. 21C: Concentration, FIG. 21D: pH, FIG. 21E: Turbidity, FIG. 21F: PS80 concentration, FIG. 21G: Charged variant analysis. (W=weeks; M=months; C=degrees Celsius; e.g., “3M-25C” denotes 3 months of storage at 25° C.).

FIGS. 22A-22C show the sub-visible particle analysis for vial stability study for Batch 2 samples. FIG. 22A: Sub-visible particles≤2 μm particles/mL, FIG. 22B: Sub-visible particles≤10 μm particles/mL, FIG. 22C: Sub-visible particles≤25 μm particles/mL. (W=weeks; M=months; C=degrees Celsius; e.g., “3M-25C” denotes 3 months of storage at 25° C.).

FIGS. 23A-23F show the analytics for the frozen-storage stability study for Batch 2 samples. FIG. 23A: HMW species, FIG. 23B: LMW species, FIG. 23C: Concentration, FIG. 23D: pH, FIG. 23E: Turbidity, FIG. 23F: Sub-visible particles (≤2 μm, ≤10 μm, ≤25 μm particles/mL). (M=months).

FIGS. 24A-24E show the peptide map and Biacore binding analysis for Batch 2 samples. FIG. 24A: LC D32 Isomerization, FIG. 24B: HC M251 Oxidation, FIG. 24C: HC PENNYK Deamidation, FIG. 24D: HC VSNK Deamidation, FIG. 24E: Biacore analysis. (V=vial; P=PFS) (W=weeks; M=months; C=degrees Celsius; e.g., “3M-25C” denotes 3 months of storage at 25° C.).

FIGS. 25A-25G show the analytics from PFS stability study for Batch 3 samples. FIG. 25A: HMW species, FIG. 25B: LMW species, FIG. 25C: Concentration, FIG. 25D: pH, FIG. 25E: Turbidity, FIG. 25F: PS80 concentration, FIG. 25G: Sub-visible particles (≤2 μm particles/mL, ≤10 μm particles/mL≤25 μm particles/mL). (W=weeks; M=months; C=degrees Celsius; e.g., “3M-25C” denotes 3 months of storage at 25° C.).

FIGS. 26A-26G show the analytics from vial stability study for Batch 3 samples. FIG. 26A: HMW species, FIG. 26B: LMW species, FIG. 26C: Concentration, FIG. 26D: pH, FIG. 26E: Turbidity, FIG. 26F: PS80 concentration, FIG. 26G: Sub-visible particles (≤2 μm particles/mL, ≤10 μm particles/mL ≤25 μm particles/mL). (W=weeks; M=months; C=degrees Celsius; e.g., “3M-25C” denotes 3 months of storage at 25° C.).

FIGS. 27A-27E show the peptide map and Biacore binding analysis for Batch 3 samples. FIG. 27A: LC D32 Isomerization, FIG. 27B: HC M251 Oxidation, FIG. 27C: HC PENNYK Deamidation, FIG. 27D: HC VSNK Deamidation, FIG. 27E: Biacore analysis. (V=vial; P=PFS). (W=weeks; M=months; C=degrees Celsius; e.g., “3M-25C” denotes 3 months of storage at 25° C.).

FIGS. 28A-28G show the analytics from vial stability study for Batch 4 samples. FIG. 28A: HMW species, FIG. 28B: LMW species, FIG. 28C: Concentration, FIG. 28D: pH, FIG. 28E: Turbidity, FIG. 28F: PS80 concentration, FIG. 28G: Sub-visible particles (≤2 μm particles/mL, ≤10 μm particles/mL ≤25 μm particles/mL). (W=weeks; M=months; C=degrees Celsius; e.g., “3M-25C” denotes 3 months of storage at 25° C.).

FIGS. 29A-29E show the peptide map and Biacore binding analysis for Batch 4 samples. FIG. 29A: LC D32 Isomerization, FIG. 29B: HC M251 Oxidation, FIG. 29C: HC PENNYK Deamidation, FIG. 29D: HC VSNK Deamidation, FIG. 29E: Biacore analysis. (V=vial). (W=weeks; M=months; C=degrees Celsius; e.g., “1M-5C” denotes 1 month of storage at 5° C.).

FIGS. 30A-30G show the analytics from vial stability study for Batch 5 samples. FIG. 30A: HMW species, FIG. 30B: LMW species, FIG. 30C: Concentration, FIG. 30D: pH, E: Turbidity, FIG. 30F: PS80 concentration, FIG. 30G: Sub-visible particles (≤2 μm particles/mL, ≤10 μm particles/mL≤25 μm particles/mL). (W=weeks; M=months; C=degrees Celsius; e.g., “1M-5C” denotes 1 month of storage at 5° C.).

FIGS. 31A-31E show the analysis of samples for wrist action shaking in vials. FIG. 31A: HMW species, FIG. 31B:LMW species, FIG. 31C: Turbidity, FIG. 31D: Charge variant analysis (% Acidic, % Monomer, % Basic), FIG. 31E: Sub-visible particles (≤2 μm, ≤10 μm, ≤25 μm particles/mL). (h=hours).

FIGS. 32A-32E show the analysis of samples from wrist action shaking in PFS. FIG. 32A: HMW species, FIG. 32B: LMW species, FIG. 32C: Turbidity, FIG. 32D: Charge variant analysis (% Acidic, % Monomer, % Basic), FIG. 32E: Sub-visible particles (≤2 μm, ≤10 μm, ≤25 μm particles/mL). (h=hours).

FIGS. 33A-33F show the analysis of samples from freeze-thaw cycling. FIG. 33A: HMW species, FIG. 33B:LMW species, FIG. 33C:Turbidity, FIG. 33D: Concentration, FIG. 33E: pH, FIG. 33F: Sub-visible particles (≤2 μm, ≤10 μm, ≤25 μm particles/mL). (FT=freeze thaw cycles).

FIGS. 34A-34I show the analysis of samples for photostability. FIG. 34A: HMW species, FIG. 34B: LMW species, FIG. 34C: Turbidity, FIG. 34D: Concentration, FIG. 34E: pH, F:PS 80 concentration, FIG. 34G: Charge variant analysis, FIG. 34H: Total peptide map analysis, FIG. 34I: Sub-visible particles (≤2 μm, ≤10 μm, ≤25 μm particles/mL). (d=days).

### DETAILED DESCRIPTION

### Humanized Anti-C1s Antibodies

The compositions of the present disclosure comprise a humanized anti-C1s antibody. A humanized anti-C1s antibody of the disclosure binds to and inhibits the activated form of C1s within the classical pathway (CP). The complement system is a component of the innate immune system that mediates humoral immunity. The mechanism of the antibody is specific to the CP, leaving the lectin and the alternative pathways functionally intact.

In some embodiments, a humanized anti-C1s antibody of the present disclosure inhibits C1s-mediated cleavage of complement component C4, e.g., by inhibiting enzymatic activity of the serine-protease domain of C1s. In some embodiments, a humanized anti-C1s antibody of the present disclosure inhibits C1s-mediated cleavage of complement component C2. In some embodiments, a humanized anti-C1s antibody of the present disclosure inhibits C1s-mediated cleavage of C4 and C2.

In some embodiments, a humanized anti-C1s antibody of the present disclosure binds a complement C1s protein having the amino acid sequence depicted in SEQ ID NO: 15. Amino acid sequence SEQ ID NO: 15, provided below, represents the Homo sapiens complement C1s protein:

(SEQ ID NO: 15)
EPTMYGEILSPNYPQAYPSEVEKSWDIEVPEGYGIHLYFTHLDIELSENC
AYDSVQIISGDTEEGRLCGQRSSNNPHSPIVEEFQVPYNKLQVIFKSDFS
NEERFTGFAAYYVATDINECTDFVDVPCSHFCNNFIGGYFCSCPPEYFLH
DDMKNCGVNCSGDVFTALIGEIASPNYPKPYPENSRCEYQIRLEKGFQVV
VTLRREDEDVEAADSAGNCLDSLVFVAGDRQFGPYCGHGFPGPLNIETKS
NALDIIFQTDLTGQKKGWKLRYHGDPMPCPKEDTPNSVWEPAKAKYVFRD
VVQITCLDGFEVVEGRVGATSFYSTCQSNGKWSNSKLKCQPVDCGIPESI
ENGKVEDPESTLFGSVIRYTCEEPYYYMENGGGGEYHCAGNGSWVNEVLG
PELPKCVPVCGVPREPFEEKQRIIGGSDADIKNFPWQVFFDNPWAGGALI
NEYWVLTAAHVVEGNREPTMYVGSTSVQTSRLAKSKMLTPEHVFIHPGWK
LLEVPEGRTNFDNDIALVRLKDPVKMGPTVSPICLPGTSSDYNLMDGDLG
LISGWGRTEKRDRAVRLKAARLPVAPLRKCKEVKVEKPTADAEAYVFTPN
MICAGGEKGMDSCKGDSGGAFAVQDPNDKTKFYAAGLVSWGPQCGTYGLY
TRVKNYVDWIMKTMQENSTPRED.

In some embodiments, a humanized anti-C1s antibody of the present disclosure binds a human complement C1s protein with a dissociation constant (KD) of no more than 2.5 nM. In some embodiments, a humanized anti-C1s antibody of the present disclosure binds a human complement C1s protein with a KD of no more than 2 nM. In some embodiments, a humanized anti-C1s antibody of the present disclosure binds a human complement C1s protein with a KD of no more than 1 nM. In some embodiments, a humanized anti-C1s antibody of the present disclosure binds a human complement C1s protein with a KD of no more than 0.9 nM, no more than 0.8 nM, no more than 0.7 nM, no more than 0.6 nM, no more than 0.5 nM, no more than 0.4 nM, no more than 0.3 nM, no more than 0.2 nM, or no more than 0.1 nM. In some embodiments, a humanized anti-C1s antibody of the present disclosure binds a human complement C1s protein with a KD of no more than 0.3 nM. In some embodiments, a humanized anti-C1s antibody of the present disclosure binds a human complement C1s protein with a KD of no more than 0.2 nM. In some embodiments, a humanized anti-C1s antibody of the present disclosure binds a human complement C1s protein with a KD of no more than 0.1 nM. Methods to measure binding of an antibody to human complement C1s protein can be determined by one skilled in the art.

In some embodiments, a humanized anti-C1s antibody of the present disclosure binds a human complement C1s protein with a KD of no more than 90 pM, no more than 80 pM, no more than 70 pM, no more than 60 pM, no more than 50 pM, no more than 40 pM, no more than 30 pM, no more than 20 pM, no more than 10 pM, no more than 9 pM, no more than 8 pM, no more than 7 pM, no more than 6 pM, no more than 5 pM, no more than 4 pM, no more than 3 pM, no more than 2 pM, or no more than 1 pM.

In some embodiments, a humanized anti-C1s antibody of the present disclosure inhibits the classical complement pathway with a half-maximal inhibitory concentration (IC₅₀) of 10⁻⁸ M or less, 5×10⁻⁹ M or less, or 10⁻⁹ M or less.

“Antibody” encompasses antibodies or immunoglobulins of any isotype, including but not limited to humanized antibodies and chimeric antibodies. An antibody may be a single-chain antibody (scAb) or a single domain antibody (dAb) (e.g., a single domain heavy chain antibody or a single domain light chain antibody; see Holt et al. (2003) Trends Biotechnol. 21:484). The term “antibody” also encompasses fragments of antibodies (antibody fragments) that retain specific binding to an antigen. “Antibody” further includes single-chain variable fragments (scFvs), which are fusion proteins of the variable regions of the heavy (V_{H}) and light chains (V_{L}) of antibodies, connected with a short linker peptide, and diabodies, which are noncovalent dimers of scFv fragments that include the V_{H} and V_{L} connected by a small peptide linker (Zapata et al., Protein Eng. 8(10): 1057-1062 (1995)). Other fusion proteins that comprise an antigen-binding portion of an antibody and a non-antibody protein are also encompassed by the term “antibody.”

“Antibody fragments” comprise a portion of an intact antibody, for example, the antigen binding or variable region of the intact antibody. Examples of antibody fragments include an antigen-binding fragment (Fab), Fab′, F(ab′)₂, a variable domain Fv fragment (Fv), an Fd fragment, and an antigen binding fragment of a chimeric antigen receptor.

Papain digestion of antibodies produces two identical antigen-binding fragments, referred to as “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment that contains a complete antigen-recognition and -binding site. This region includes a dimer of one heavy-chain variable domain and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Fab” fragments contain the constant domain of the light chain and the first constant domain (CH₁) of the heavy chain. Fab fragments differ from Fab′ fragments by the addition of a few residues at the carboxyl terminus of the heavy chain CH₁ domain including at least one cysteine from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“scFv” antibody fragments comprise the V_{H} and V_{L} of an antibody, wherein these regions are present in a single polypeptide chain. In some embodiments, the Fv polypeptide further comprises a polypeptide linker between the V_{H} and V_{L} regions, which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

“Diabody” refers to a small antibody fragment with two antigen-binding sites, which fragments comprise a V_{H} connected to a V_{L} in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, Hollinger et al. Proc. Natl. Acad. Sci. USA 90: 6444-6448 (1993).

An antibody can be monovalent or bivalent. An antibody can be an Ig monomer, which is a “Y-shaped” molecule that consists of four polypeptide chains: two heavy chains and two light chains connected by disulfide bonds.

Antibodies can be detectably labeled, e.g., with a radioisotope, an enzyme that generates a detectable product, and/or a fluorescent protein. Antibodies can be further conjugated to other moieties, such as members of specific binding pairs, e.g., biotin member of biotin-avidin specific binding pair. Antibodies can also be bound to a solid support, including, but not limited to, polystyrene plates and/or beads.

An “isolated” antibody is one that has been identified and separated and/or recovered from a component of its natural environment (i.e., is not naturally occurring). Contaminant components of its natural environment are materials that would interfere with uses (e.g., diagnostic or therapeutic uses) of the antibody, and can include enzymes, hormones, and other proteinaceous or nonproteinaceous solutes. In some embodiments, an antibody is purified (1) to greater than 90%, greater than 95%, greater than 98% or greater than 99% by weight of antibody as determined by the Lowry method, for example, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or non-reducing conditions using Coomassie blue or silver stain. Isolated antibodies encompass antibodies in situ within recombinant cells, as at least one component of the antibody's natural environment will not be present. In some embodiments, an isolated antibody is prepared by at least one purification step.

A “monoclonal antibody” is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries).

A “complementarity determining region (CDR)” is the non-contiguous antigen combining sites found within the variable region of both heavy and light chain polypeptides. CDRs have been described by Lefranc et al. (2003) Developmental and Comparative Immunology 27:55; Kabat et al., J. Biol. Chem. 252:6609-6616 (1977); Kabat et al., U. S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of any of the Kabat, Lefranc, Chothia, or MacCallum definitions (also referred to as numbering systems) to refer to a CDR of an antibody or grafted antibodies or variants thereof is intended to be within the scope of the term as defined and used herein.

The terms “LC CDR1,” “LC CDR2,” and “LC CDR3” refer, respectively, to the first, second, and third CDRs in a light chain variable region. As used herein, the terms “HC CDR1,” “HC CDR2,” and “HC CDR3” refer, respectively, to the first, second, and third CDRs in a heavy chain variable region. As used herein, the terms “CDR1,” “CDR2,” and “CDR3” refer, respectively, to the first, second and third CDRs of either chain's variable region.

A “framework” when used in reference to an antibody variable region includes all amino acid residues outside the CDR regions within the variable region of an antibody. A variable region framework is generally a discontinuous amino acid sequence that includes only those amino acids outside of the CDRs. A “framework region” includes each domain of the framework that is separated by the CDRs.

A “humanized antibody” is an antibody comprising portions of antibodies of different origin, wherein at least one portion comprises amino acid sequences of human origin. For example, the humanized antibody can comprise portions derived from an antibody of nonhuman origin with the requisite specificity, such as a mouse, and from antibody sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized antibody is an antibody containing at least one chain comprising a CDR derived from an antibody of nonhuman origin and a framework region derived from a light and/or heavy chain of human origin (e.g., CDR-grafted antibodies with or without framework changes). Chimeric or CDR-grafted single chain antibodies are also encompassed by the term humanized immunoglobulin. See, e.g., Cabilly et al., U. S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1. See also, Ladner et al., U.S. Pat. No. 4,946,778; Huston, U.S. Pat. No. 5,476,786; and Bird, R. E. et al., Science, 242: 423-426 (1988)), regarding single chain antibodies.

In some embodiments, a humanized antibody is produced using synthetic and/or recombinant nucleic acids to prepare genes (e.g., cDNA) encoding the desired humanized chain. For example, nucleic acid (e.g., DNA) sequences coding for humanized variable regions can be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. For example, cloned variable regions can be mutagenized, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993).

In some embodiments, a humanized anti-C1s antibody described herein is a full-length IgG, an Ig monomer, a Fab fragment, a F(ab′)₂ fragment, a Fd fragment, a scFv, a scAb, or a Fv. In some embodiments, a humanized anti-C1s antibody described herein is a full-length IgG. In some embodiments, the heavy chain of any of the humanized anti-C1s antibodies as described herein comprises a heavy chain constant region (CH) or a portion thereof (e.g., CH1, CH2, CH3, or a combination thereof). The heavy chain constant region can be of any suitable origin, e.g., human, mouse, rat, or rabbit. In some embodiments, the heavy chain constant region is from a human IgG (a gamma heavy chain), e.g., IgG1, IgG2, or IgG4.

In some embodiments, mutations can be introduced into the heavy chain constant region of any one of the humanized anti-C1s antibodies described herein. In some embodiments, one, two or more mutations (e.g., amino acid substitutions) are introduced into the heavy chain constant region (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more mutations (e.g., amino acid substitutions) are introduced into the hinge region of the heavy chain constant region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more amino acid mutations (i.e., substitutions, insertions or deletions) are introduced into an IgG constant domain, or FcRn-binding fragment thereof to alter (e.g., decrease or increase) half-life of the antibody in vivo. In some embodiments, the one or more mutations are introduced into an Fc or hinge-Fc domain fragment. See, e.g., International Publication Nos. WO 02/060919; WO 98/23289; and WO 97/34631; and U.S. Pat. Nos. 5,869,046; 6,121,022; 6,277,375; and 6,165,745 for examples of mutations that will alter (e.g., decrease or increase) the half-life of an antibody in vivo.

In some embodiments, the constant region antibody described herein is an IgG1 constant region and comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat. Additional mutations that may be introduced to the heavy chain constant region that would increase the half-life of the antibody are known in the art, e.g., the M428L/N434S (EU numbering; M459L/N466S Kabat numbering) mutations as described in Zalevsky et al., Nat Biotechnol. 2010 Feb; 28(2): 157-159.

In some embodiments, one, two or more amino acid substitutions are introduced into an IgG constant domain Fc region to alter the effector function(s) of the antibody. The effector ligand to which affinity is altered can be, for example, an Fc receptor or the C1 component of complement. This approach is described in further detail in U.S. Pat. Nos. 10 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, at least one amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, at least one amino acid in the constant region can be replaced with a different amino acid residue such that the antibody has altered C1q binding and/or reduced or abolished complement dependent cytotoxicity (CDC). This approach is described in further detail in U.S. Pat. No. 6,194,551 (Idusogie et al.). In some embodiments, at least one amino acid residue in the N-terminal region of the CH2 domain of an antibody described herein is altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or to increase the affinity of the antibody for an Fcγ receptor. This approach is described further in International Publication No. WO 00/42072.

In some embodiments, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. In some embodiments, to reduce residual antibody-dependent cellular cytotoxicity, a L235E (EU numbering, corresponding to L248E in Kabat numbering) mutation is introduced to the heavy chain constant region, e.g., as described in Benhnia et al., J. Virology, Dec. 2009, p. 12355-12367.

In some embodiments, a humanized anti-C1s antibody comprises a light chain complementarity determining region 1 (LC CDR1) comprising the amino acid sequence of KASQSVDYDGDSYMN (SEQ ID NO: 1). In some embodiments, a humanized anti-C1s antibody comprises a light chain complementarity determining region 2 (LC CDR2) comprising the amino acid sequence of DASNLES (SEQ ID NO: 2). In some embodiments, a humanized anti-C1s antibody comprises a light chain complementarity determining region 3 (LC CDR3) comprising the amino acid sequence of QQSNEDPWT (SEQ ID NO: 3). In some embodiments, a humanized anti-C1s antibody comprises an LC CDR1 comprising the amino acid sequence of SEQ ID NO: 1, an LC CDR2 comprising the amino acid sequence of SEQ ID NO: 2, and an LC CDR3 comprising the amino acid sequence of SEQ ID NO: 3.

In some embodiments, a humanized anti-C1s antibody comprises a heavy chain complementarity determining region 1 (HC CDR1) comprising the amino acid sequence of DDYIH (SEQ ID NO: 4). In some embodiments, a humanized anti-C1s antibody comprises a heavy chain complementarity determining region 2 (HC CDR2) comprising the amino acid sequence of RIDPADGHTKYAPKFQV (SEQ ID NO: 5). In some embodiments, a humanized anti-C1s antibody comprises a heavy chain complementarity determining region 3 (HC CDR3) comprising the amino acid sequence of YGYGREVFDY (SEQ ID NO: 6). In some embodiments, a humanized anti-C1s antibody comprises an HC CDR1 comprising the amino acid sequence of SEQ ID NO: 4, an HC CDR2 comprising the amino acid sequence of SEQ ID NO: 5, and an HC CDR3 comprising the amino acid sequence of SEQ ID NO: 6.

In some embodiments, a humanized anti-C1s antibody comprises an LC CDR1 that comprises the amino acid sequence of SEQ ID NO: 1, an LC CDR2 comprising the amino acid sequence of SEQ ID NO: 2, and an LC CDR3 comprising the amino acid sequence of SEQ ID NO: 3, an HC CDR1 comprising the amino acid sequence of SEQ ID NO: 4, an HC CDR2 comprising the amino acid sequence of SEQ ID NO: 5, an HC CDR3 comprising the amino acid sequence of SEQ ID NO: 6.

In some embodiments, a humanized anti-C1s antibody comprises a light chain variable region (VL) comprising the amino acid sequence of DIVLTQSPDSLAVSLGERATISCKASQSVDYDGDSYMNWYQQKPGQPPKILIYDASN LESGIPARFSGSGSGTDFTLTISSLEPEDFAIYYCQQSNEDPWTFGGGTKVEIK (SEQ ID NO: 7). In some embodiments, a humanized anti-C1s antibody comprises a LC CDR1, LC CDR2, and LC CDR3 of a VL comprising the amino acid sequence of SEQ ID NO: 7.

In some embodiments, a humanized anti-C1s antibody comprises a heavy chain variable region (VH) comprising the amino acid sequence of QVQLVQSGAEVKKPGASVKLSCTASGFNIKDDYIHWVKQAPGQGLEWIGRIDPADG HTKYAPKFQVKVTITADTSTSTAYLELSSLRSEDTAVYYCARYGYGREVFDYWGQG TTVTVSS (SEQ ID NO: 8). HC CDR1, HC CDR2, and HC CDR3 of a VH comprising the amino acid sequence of SEQ ID NO: 8.

In some embodiments, a humanized anti-C1s antibody comprises a VL comprising the amino acid sequence of SEQ ID NO: 7 and a VH comprising the amino acid sequence of SEQ ID NO: 8.

In some embodiments, a humanized anti-C1s antibody comprises a LC CDR1, LC CDR2, and LC CDR3 of a VL comprising the amino acid sequence of SEQ ID NO: 7 and HC CDR1, HC CDR2, and HC CDR3 of a VH comprising the amino acid sequence of SEQ ID NO: 8.

In some embodiments, a humanized anti-C1s antibody comprises a light chain (LC) comprising the amino acid sequence of

(SEQ ID NO: 9)
DIVLTQSPDSLAVSLGERATISCKASQSVDYDGDSYMNWYQQKPGQPPKI
LIYDASNLESGIPARFSGSGSGTDFTLTISSLEPEDFAIYYCQQSNEDPW
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

In some embodiments, a humanized anti-C1s antibody comprises a heavy chain (HC) comprising the amino acid sequence of

(SEQ ID NO: 10)
QVQLVQSGAEVKKPGASVKLSCTASGFNIKDDYIHWVKQAPGQGLEWIGR
IDPADGHTKYAPKFQVKVTITADTSTSTAYLELSSLRSEDTAVYYCARYG
YGREVEDYWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFEGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVLHEALHSHYTQKSLSLSLGK.

In some embodiments, a humanized anti-C1s antibody comprises a LC comprising the amino acid sequence of SEQ ID NO: 9 and a HC comprising the amino acid sequence of SEQ ID NO: 10.

In some embodiments, a humanized anti-C1s antibody comprises an LC CDR1 comprising an amino acid sequence containing no more than 3 amino acid variations (e.g., no more than 3, 2, or 1 amino acid variation(s)) relative to the LC CDR1 amino acid sequence of SEQ ID NO: 1. In some embodiments, a humanized anti-C1s antibody comprises an LC CDR2 comprising an amino acid sequence containing no more than 3 amino acid variations (e.g., no more than 3, 2, or 1 amino acid variation(s)) relative to the LC CDR2 amino acid sequence of SEQ ID NO: 2. In some embodiments, a humanized anti-C1s antibody comprises an LC CDR3 comprising an amino acid sequence containing no more than 3 amino acid variations (e.g., no more than 3, 2, or 1 amino acid variation(s)) relative to the LC CDR3 amino acid sequence of SEQ ID NO: 3.

In some embodiments, a humanized anti-C1s antibody comprises an HC CDR1 comprising an amino acid sequence containing no more than 3 amino acid variations (e.g., no more than 3, 2, or 1 amino acid variation(s)) relative to the HC CDR1 amino acid sequence of SEQ ID NO: 4. In some embodiments, a humanized anti-C1s antibody comprises an HC CDR2 comprising an amino acid sequence containing no more than 3 amino acid variations (e.g., no more than 3, 2, or 1 amino acid variation(s)) relative to the HC CDR2 amino acid sequence of SEQ ID NO: 5. In some embodiments, a humanized anti-C1s antibody comprises an HC CDR3 comprising an amino acid sequence containing no more than 3 amino acid variations (e.g., no more than 3, 2, or 1 amino acid variation(s)) relative to the HC CDR3 amino acid sequence of SEQ ID NO: 6.

In some embodiments, a humanized anti-C1s antibody comprises a VL comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VL amino acid sequence of SEQ ID NO: 7.

In some embodiments, a humanized anti-C1s antibody comprises a VH comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VH amino acid sequence of SEQ ID NO: 8.

In some embodiments, a humanized anti-C1s antibody comprises a VL comprising an LC CDR1 comprising the amino acid sequence of SEQ ID NO: 1, an LC CDR2 comprising the amino acid sequence of SEQ ID NO: 2, an LC CDR3 comprising the amino acid sequence of SEQ ID NO: 3, and comprises framework regions that contain no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VL sequence of SEQ ID NO: 7.

In some embodiments, a humanized anti-C1s antibody comprises a VH comprising an HC CDR1 comprising the amino acid sequence of SEQ ID NO: 4, an HC CDR2 comprising the amino acid sequence of SEQ ID NO: 5, an HC CDR3 comprising the amino acid sequence of SEQ ID NO: 6, and comprises framework regions that contain no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VH sequence of SEQ ID NO: 8.

In some embodiments, a humanized anti-C1s antibody comprises (a) a VL comprising an LC CDR1 comprising the amino acid sequence of SEQ ID NO: 1, an LC CDR2 comprising the amino acid sequence of SEQ ID NO: 2, an LC CDR3 comprising the amino acid sequence of SEQ ID NO: 3, and comprises framework regions that contain no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VL sequence of SEQ ID NO: 7, and (b) a VH comprising an HC CDR1 comprising the amino acid sequence of SEQ ID NO: 4, an HC CDR2 comprising the amino acid sequence of SEQ ID NO: 5, an HC CDR3 comprising the amino acid sequence of SEQ ID NO: 6, and comprises framework regions that contain no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VH sequence of SEQ ID NO: 8.

In some embodiments, a humanized anti-C1s antibody comprises a VL comprising an amino acid sequence having at least 80% (e.g., 80%, 85%, 90%, 95%, 96%, 97%, 98%, or 99%) identity to the VL amino acid sequence of SEQ ID NO: 7.

In some embodiments, a humanized anti-C1s antibody comprises a VH comprising an amino acid sequence having at least 80% (e.g., 80%, 85%, 90%, 95%, 96%, 97%, 98%, or 99%) identity to the VH amino acid sequence of SEQ ID NO: 8.

In some embodiments, a humanized anti-C1s antibody comprises a VL comprising an LC CDR1 comprising the amino acid sequence of SEQ ID NO: 1, an LC CDR2 comprising the amino acid sequence of SEQ ID NO: 2, an LC CDR3 comprising the amino acid sequence of SEQ ID NO: 3 and comprises framework regions that have at least 80% (e.g., 80%, 85%, 90%, 95%, 96%, 97%, 98%, or 99%) identity to the framework regions of the VL sequence of SEQ ID NO: 7.

In some embodiments, a humanized anti-C1s antibody comprises a VH comprising an HC CDR1 comprising the amino acid sequence of SEQ ID NO: 4, an HC CDR2 comprising the amino acid sequence of SEQ ID NO: 5, an HC CDR3 comprising the amino acid sequence of SEQ ID NO: 6 and comprises framework regions that have at least 80% (e.g., 80%, 85%, 90%, 95%, 96%, 97%, 98%, or 99%) identity to the framework regions of the VH sequence of SEQ ID NO: 8.

In some embodiments, a humanized anti-C1s antibody comprises (a) a VL comprising an LC CDR1 comprising the amino acid sequence of SEQ ID NO: 1, an LC CDR2 comprising the amino acid sequence of SEQ ID NO: 2, an LC CDR3 comprising the amino acid sequence of SEQ ID NO: 3, and comprises framework regions that have at least 80% (e.g., 80%, 85%, 90%, 95%, 96%, 97%, 98%, or 99%) identity to the framework regions of the VL sequence of SEQ ID NO: 7, and (b) a VH comprising an HC CDR1 comprising the amino acid sequence of SEQ ID NO: 4, an HC CDR2 comprising the amino acid sequence of SEQ ID NO: 5, an HC CDR3 comprising the amino acid sequence of SEQ ID NO: 6, and comprises framework regions that have at least 80% (e.g., 80%, 85%, 90%, 95%, 96%, 97%, 98%, or 99%) identity to the framework regions of the VH sequence of SEQ ID NO: 8.

In some embodiments, the heavy chain constant region in any one of the humanized anti-C1s antibodies described herein is an IgG4 constant region, or a variant there of. Examples of IgG4 constant regions and variants are provided in Table 1.

TABLE 1
Examples of Heavy Chain Constant Regions

| Heavy Chain | |
|---|---|
| Constant | |
| Region | Amino Acid Sequence |
| IgG4 constant | ASTKGPSVFPLAPCSRSTSESTAALGCLVKDYFPEPVTVSWNSGALTSGVHTFPAVLOSSG |
| region WT | LYSLSSVVTVPSSSLGTKTYTCNVDHKPSNTKVDKRVESKYGPPCPSCPAPEFLGGPSVFL |
| | FPPKPKDTLMISRTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPSQEEMTKNQVS |
| | LTCLVKGFYPSDIAVEWESNGQPENNYKTTPPVLDSDGSFFLYSRLTVDKSRWQEGNVFSC |
| | SVMHEALHNHYTQKSLSLSLGK (SEQ ID NO: 11) |
| IgG4 constant | ASTKGPSVFPLAPCSRSTSESTAALGCLVKDYFPEPVTVSWNSGALTSGVHTFPAVLQSSG |
| region variant 1 | LYSLSSVVTVPSSSLGTKTYTCNVDHKPSNTKVDKRVESKYGPPCPPCPAPEFEGGPSVEL |
| | FPPKPKDTLMISRTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPSQEEMTKNQVS |
| | LTCLVKGFYPSDIAVEWESNGQPENNYKTTPPVLDSDGSFFLYSRLTVDKSRWQEGNVFSC |
| | SVMHEALHNHYTQKSLSLSLGK (SEQ ID NO: 12) |
| IgG4 constant | ASTKGPSVFPLAPCSRSTSESTAALGCLVKDYFPEPVTVSWNSGALTSGVHTFPAVLQSSG |
| region variant 2 | LYSLSSVVTVPSSSLGTKTYTCNVDHKPSNTKVDKRVESKYGPPCPPCPAPEFEGGPSVEL |
| | FPPKPKDTLMISRTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPSQEEMTKNQVS |
| | LTCLVKGFYPSDIAVEWESNGQPENNYKTTPPVLDSDGSFFLYSRLTVDKSRWQEGNVFSC |
| | SVLHEALHSHYTQKSLSLSLGK (SEQ ID NO: 13) |

In some embodiments, the light chain of any of the humanized anti-C1s antibodies described herein may further comprise a light chain constant region (C_{L}). In some examples, the C_{L} is a kappa light chain. In other examples, the C_{L} is a lambda light chain. In some embodiments, the C_{L} is a kappa light chain, the sequence of which is provided below:

(SEQ ID NO: 14)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SENRGEC

Other antibody heavy and light chain constant regions are well known in the art, e.g., those provided in the IMGT database (imgt.org) or at vbase2.org/vbstat.php, both of which are incorporated by reference herein.

### Compositions

The present disclosure provides compositions, including pharmaceutical compositions or formulations, comprising a humanized anti-C1s antibody. A humanized anti-C1s antibody of the present disclosure can be formulated into pharmaceutical compositions by combination with appropriate pharmaceutically acceptable carriers, pharmaceutically acceptable diluents, or other pharmaceutically acceptable excipients.

Exemplary antibody concentrations in a composition of the disclosure can range from about 50 mg/mL to about 250 mg/mL, about 75 mg/mL to about 225 mg/mL, about 100 mg/mL to about 200 mg/mL, about 100 mg/mL to about 150 mg/mL, about 150 mg/mL to about 200 mg/mL, about 125 mg/mL to about 175 mg/mL, or about 140 mg/mL to about 160 mg/mL. In some embodiments, the antibody concentration in a composition of the disclosure is about 50 mg/mL, about 75 mg/mL, about 100 mg/mL, about 125 mg/mL, about 130 mg/mL, about 140 mg/mL, about 150 mg/mL, about 160 mg/mL, about 170 mg/mL, about 175 mg/mL, about 200 mg/mL, or about 250 mg/mL. In some embodiments, a composition of the disclosure comprises about 150 mg/mL of the humanized anti-C1s antibody.

In some embodiments, a composition of the disclosure comprises arginine or a salt thereof. Arginine may function as a stabilizer. As described herein, the presence of arginine reduces the rate of isomerization of the aspartic acid residue at position number 32 in the light chain variable domain sequence (D32 isomerization rate) of the subject antibody. Exemplary concentrations of arginine or a salt thereof in a composition can range from about 50 mM to about 200 mM, about 75 mM to about 175 mM, about 100 mM to about 150 mM, about 125 mM to about 175 mM, or about 125 mM to about 150 mM. In some embodiments, a composition of the disclosure comprises about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, or about 200 mM arginine or a salt thereof. Exemplary salts of arginine include arginine citrate, arginine hydrochloride, arginine oxalate, arginine phosphate, arginine succinate, or arginine tartrate. Other salts of arginine may also be used. In some embodiments, a composition of the disclosure comprises arginine hydrochloride. In some embodiments, a composition of the disclosure comprises about 50 mM to about 200 mM, about 75 mM to about 175 mM, about 100 mM to about 150 mM, about 125 mM to about 175 mM, or about 125 mM to about 150 mM of arginine hydrochloride. In some embodiments, a composition of the disclosure comprises about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, or about 200 mM arginine hydrochloride. In some embodiments, a composition of the disclosure comprises about 150 mM arginine hydrochloride. In some embodiments, the arginine hydrocholoride is L-arginine hydrochloride.

In some embodiments, a composition of the disclosure comprises a buffering agent. Exemplary concentrations of a buffering agent in a composition can range from about 1 mM to about 50 mM, about 10 mM to about 40 mM, about 10 mM to about 30 mM, about 10 mM to about 25 mM, about 10 mM to about 25 mM, about 5 mM to about 25 mM, about 5 mM to about 20 mM, or about 5 mM to about 10 mM. In some embodiments, a composition of the disclosure comprises about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM of a buffering agent. Exemplary buffering agents include acetate, citrate, histidine, oxalate, phosphate, succinate, and tartrate. Other buffering agents may also be used. In some embodiments, the buffering agent is histidine. In some embodiments, a composition of the disclosure comprises about 1 mM to about 50 mM, about 10 mM to about 40 mM, about 10 mM to about 30 mM, about 10 mM to about 25 mM, about 10 mM to about 25 mM, about 5 mM to about 25 mM, about 5 mM to about 20 mM, or about 5 mM to about 10 mM histidine. In some embodiments, a composition of the disclosure comprises about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM of histidine. In some embodiments, a composition of the disclosure comprises about 10 mM histidine. The histidine buffer may comprise histidine and a histidine salt. The concentration of histidine refers to the concentration of histidine and any optional salt of histidine that is present. The contribution of individual components to the concentration depends on the target pH. In some embodiments, the histidine buffer comprises histidine and a histidine hydrochloride. In some embodiments, the concentration of histidine refers to the concentration of both histidine and histidine hydrochloride.

In some embodiments, a composition of the disclosure comprises a stabilizer in addition to arginine. Exemplary concentrations of a stabilizer in a composition can range from about 1% to about 8% (w/v), about 1% to about 5% (w/v), about 1% to about 3% (w/v), or about 3% to about 5% (w/v). In some embodiments, a composition of the disclosure comprises about 1% (w/v), about 2% (w/v), about 3% (w/v), about 4% (w/v), about 5% (w/v), about 6% (w/v), about 7% (w/v), or about 8% (w/v) of a stabilizer. In some embodiments, the stabilizer is a sugar, sugar alcohol, or an amino sugar. Exemplary sugars, sugar alcohols, and amino sugars include fructose, galactose, glucose, lactose, maltose, mannose, raffinose, sorbitol, sorbose, sucrose, galactosamine, glucosamine, N-methylglucosamine, and neuraminic acid. Other stabilizers may also be used. In some embodiments, a composition of the disclosure comprises sucrose, trehalose, or sorbitol. In some embodiments, a composition of the disclosure comprises sucrose. In some embodiments, a composition of the disclosure comprises about 1% to about 8% (w/v), about 1% to about 5% (w/v), about 1% to about 3% (w/v), or about 3% to about 5% (w/v) of sucrose. In some embodiments, a composition of the disclosure comprises about 1% (w/v), about 2% (w/v), about 3% (w/v), about 4% (w/v), about 5% (w/v), about 6% (w/v), about 7% (w/v), or about 8% (w/v) of sucrose. In some embodiments, a composition of the disclosure comprises about 3% (w/v) sucrose. Sucrose may also function as a cryoprotectant. In some embodiments, a composition may comprise a different cryoprotectant selected from ethylene glycol, dimethyl sulfoxide (DMSO), glycerol, trehalose, and propylene glycol. Other cryoprotectants may also be used. In some embodiments, the concentration of a cryoprotectant in a composition can range from about 1% to about 10% (w/v), about 1% to about 5% (w/v), about 1% to about 3% (w/v), or about 3% to about 5% (w/v). In some embodiments, a composition of the disclosure comprises about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v) of a cryoprotectant.

In some embodiments, a composition of the disclosure comprises a chelator. Exemplary concentrations of a chelator in a composition can range from about 1 μM to about 50 μM, about 5 μM to about 40 μM, about 5 μM to about 30 μM, about 5 μM to about 25 μM, about 5 μM to about 20 μM, about 5 μM to about 15 μM, about 10 μM to about 20 μM, about 10 μM to about 25 μM, or about 10 μM to 30 μM. In some embodiments, a composition of the disclosure comprises a chelator at a concentration of about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, or about 20 μM. Exemplary chelators include ethylenediaminetetraacetic acid (EDTA), diethylenetriamine pentaacetate (DTPA), dihyroxy ethyl glycine, citric acid, tartaric acid, and methionine. Other chelators may also be used. In some embodiments, the chelator is methionine. In some embodiments, a composition of the disclosure comprises about 5 mM to about 10 mM, about 5 mM to about 7.5 mM, about 7.5 mM to about 10 mM, about 6 mM to about 8 mM, or about 8 mM to about 10 mM methionine. In some embodiments, a composition of the disclosure comprises about 5 mM, about 5.5 mM, about 6 mM, about 6.5 mM, about 7 mM, about 7.5 mM, about 8 mM, about 8.5 mM, about 9 mM, about 9.5 mM, or about 10 mM methionine. The skilled artisan would recognize that the concentration of a chelator may vary depending on the chelator being used. In some embodiments, the chelator is DTPA. In some embodiments, a composition of the disclosure comprises about 1 μM to about 50 μM, about 5 μM to about 40 μM, about 5 μM to about 30 μM, about 5 μM to about 25 μM, about 5μM to about 20 μM, about 5 μM to about 15 μM, about 10 μM to about 20 μM, about 10 μM to about 25 μM, or about 10 μM to 30 μM DTPA. In some embodiments, a composition of the disclosure comprises about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, or about 20 μM DTPA. In some embodiments, a composition of the disclosure comprises about 10 μM DTPA.

In some embodiments, a composition of the disclosure comprises a surfactant. Exemplary concentrations of a surfactant in a composition can range from about 0.01% to about 0.1% (w/v), about 0.03% to about 0.6% (w/v), about 0.03% to about 0.08% (w/v), about 0.03% to about 0.1% (w/v), about 0.05% to about 0.1% (w/v), or about 0.06% to about 0.1% (w/v). In some embodiments, a composition of the disclosure comprises a surfactant at a concentration of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), or about 0.1% (w/v). Exemplary surfactants include polysorbates (e.g., polysorbate 20 (PS20), polysorbate 40 (PS40), polysorbate 60 (PS60), and polysorbate 80 (PS80)), dicarboxylic acids, oxalic acid, succinic acid, fumaric acid, phthalic acid, polyoxyethylene sorbitan monooleate, poloxamers (e.g., P188), and polyethylene glycol. Other surfactants may also be used. In some embodiments, the surfactant is P188. In some embodiments, the surfactant is PS80. In some embodiments, a composition of the disclosure comprises about 0.01% to about 0.1% (w/v), about 0.03% to about 0.6% (w/v), about 0.03% to about 0.08% (w/v), about 0.03% to about 0.1% (w/v), about 0.05% to about 0.1% (w/v), about 0.06% to about 0.1% (w/v) PS80. In some embodiments, a composition of the disclosure comprises about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), or about 0.1% (w/v) PS80. In some embodiments, a composition of the disclosure comprises about 0.06% (w/v) PS80.

A composition of the disclosure may have a pH of from about 6 to about 7.5, about 6 to about 7, about 6.5 to about 7.5, or about 6.5 to about 7.1. In some embodiments, a composition of the disclosure has a pH of about 6.0, about 6.1, about 6.2, about 6.3, about 6.4, about 6.5, about 6.6. about 6.7, about 6.8, about 6.9, about 7.0, about 7.1, about 7.2, about 7.3, about 7.4, or about 7.5. In some embodiments, a composition of the disclosure has a pH of about 6.5-7.1. In some embodiments, a composition of the disclosure has a pH of about 6.8. The studies described herein demonstrate that adjusting the pH in compositions comprising arginine was beneficial for reducing D32 isomerization of the subject antibody. The pH of the composition may be measured by any means known to those of skill in the art. A means for measuring pH is using a pH meter with a micro-electrode. The pH of the composition may be adjusted using any means known in the art (e.g., by adding or adjusting the concentration of an acid, base, or buffering agent).

In some embodiments, the disclosure provides a composition comprising a humanized anti-C1s antibody, arginine or a salt thereof, and one or more of: a buffer, a stabilizer or cryoprotectant, a chelator, and a surfactant.

In some embodiments, the disclosure provides a composition comprising (a) about 50 mg/mL to about 250 mg/mL of a humanized anti-C1s antibody; (b) about 50 mM to about 200 mM arginine HCl; (c) about 1 mM to about 50 mM histidine; (d) about 1% to about 10% (w/v) sucrose; (e) about 1 μM to about 50 μM DTPA; and (f) about 0.01% to about 0.1% (w/v) polysorbate 80 (PS80), and wherein the composition has a pH of about 6 to about 7. In some embodiments, the arginine hydrocholoride is L-arginine hydrochloride.

In some embodiments, the disclosure provides a composition comprising (a) about 50 mg/mL to about 250 mg/mL of a humanized anti-C1s antibody; (b) about 50 mM to about 200 mM arginine citrate; (c) about 1 mM to about 50 mM histidine; (d) about 1% to about 10% (w/v) sucrose; (e) about 1 μM to about 50 μM DTPA; and (f) about 0.01% to about 0.1% (w/v) polysorbate 80 (PS80), and wherein the composition has a pH of about 6 to about 7.

In some embodiments, the disclosure provides a composition comprising (a) about 50 mg/mL to about 250 mg/mL of a humanized anti-C1s antibody; (b) about 50 mM to about 200 mM arginine oxalate; (c) about 1 mM to about 50 mM histidine; (d) about 1% to about 10% (w/v) sucrose; (e) about 1 μM to about 50 μM DTPA; and (f) about 0.01% to about 0.1% (w/v) polysorbate 80 (PS80), and wherein the composition has a pH of about 6 to about 7.

In some embodiments, the disclosure provides a composition comprising (a) about 50 mg/mL to about 250 mg/mL of a humanized anti-C1s antibody; (b) about 50 mM to about 200 mM arginine phosphate; (c) about 1 mM to about 50 mM histidine; (d) about 1% to about 10% (w/v) sucrose; (e) about 1 μM to about 50 μM DTPA; and (f) about 0.01% to about 0.1% (w/v) polysorbate 80 (PS80), and wherein the composition has a pH of about 6 to about 7.

In some embodiments, the disclosure provides a composition comprising (a) about 50 mg/mL to about 250 mg/mL of a humanized anti-C1s antibody; (b) about 50 mM to about 200 mM arginine succinate; (c) about 1 mM to about 50 mM histidine; (d) about 1% to about 10% (w/v) sucrose; (e) about 1 μM to about 50 μM DTPA; and (f) about 0.01% to about 0.1% (w/v) polysorbate 80 (PS80), and wherein the composition has a pH of about 6 to about 7.

In some embodiments, the disclosure provides a composition comprising (a) about 50 mg/mL to about 250 mg/mL of a humanized anti-C1s antibody; (b) about 50 mM to about 200 mM arginine tartrate; (c) about 1 mM to about 50 mM histidine; (d) about 1% to about 10% (w/v) sucrose; (e) about 1 μM to about 50 μM DTPA; and (f) about 0.01% to about 0.1% (w/v) polysorbate 80 (PS80), and wherein the composition has a pH of about 6 to about 7.

In some embodiments, the disclosure provides a composition comprising (a) about 50 mg/mL to about 250 mg/mL of a humanized anti-C1s antibody; (b) about 50 mM to about 200 mM arginine hydrochloride; (c) about 1 mM to about 50 mM acetate; (d) about 1% to about 10% (w/v) sucrose; (e) about 1 μM to about 50 μM DTPA; and (f) about 0.01% to about 0.1% (w/v) polysorbate 80 (PS80), and wherein the composition has a pH of about 6 to about 7. In some embodiments, the arginine hydrocholoride is L-arginine hydrochloride.

In some embodiments, the disclosure provides a composition comprising (a) about 50 mg/mL to about 250 mg/mL of a humanized anti-C1s antibody; (b) about 50 mM to about 200 mM arginine hydrochloride; (c) about 1 mM to about 50 mM citrate; (d) about 1% to about 10% (w/v) sucrose; (e) about 1 μM to about 50 μM DTPA; and (f) about 0.01% to about 0.1% (w/v) polysorbate 80 (PS80), and wherein the composition has a pH of about 6 to about 7. In some embodiments, the arginine hydrocholoride is L-arginine hydrochloride.

In some embodiments, the disclosure provides a composition comprising (a) about 50 mg/mL to about 250 mg/mL of a humanized anti-C1s antibody; (b) about 50 mM to about 200 mM arginine hydrochloride; (c) about 1 mM to about 50 mM oxalate; (d) about 1% to about 10% (w/v) sucrose; (e) about 1 μM to about 50 μM DTPA; and (f) about 0.01% to about 0.1% (w/v) polysorbate 80 (PS80), and wherein the composition has a pH of about 6 to about 7. In some embodiments, the arginine hydrocholoride is L-arginine hydrochloride.

In some embodiments, the disclosure provides a composition comprising (a) about 50 mg/mL to about 250 mg/mL of a humanized anti-C1s antibody; (b) about 50 mM to about 200 mM arginine hydrochloride; (c) about 1 mM to about 50 mM phosphate; (d) about 1% to about 10% (w/v) sucrose; (e) about 1 μM to about 50 μM DTPA; and (f) about 0.01% to about 0.1% (w/v) polysorbate 80 (PS80), and wherein the composition has a pH of about 6 to about 7. In some embodiments, the arginine hydrocholoride is L-arginine hydrochloride.

In some embodiments, the disclosure provides a composition comprising (a) about 50 mg/mL to about 250 mg/mL of a humanized anti-C1s antibody; (b) about 50 mM to about 200 mM arginine hydrochloride; (c) about 1 mM to about 50 mM succinate; (d) about 1% to about 10% (w/v) sucrose; (e) about 1 μM to about 50 μM DTPA; and (f) about 0.01% to about 0.1% (w/v) polysorbate 80 (PS80), and wherein the composition has a pH of about 6 to about 7. In some embodiments, the arginine hydrocholoride is L-arginine hydrochloride.

In some embodiments, the disclosure provides a composition comprising (a) about 50 mg/mL to about 250 mg/mL of a humanized anti-C1s antibody; (b) about 50 mM to about 200 mM arginine hydrochloride; (c) about 1 mM to about 50 mM tartrate; (d) about 1% to about 10% (w/v) sucrose; (e) about 1 μM to about 50 μM DTPA; and (f) about 0.01% to about 0.1% (w/v) polysorbate 80 (PS80), and wherein the composition has a pH of about 6 to about 7. In some embodiments, the arginine hydrocholoride is L-arginine hydrochloride.

In some embodiments, the disclosure provides a composition comprising (a) about 50 mg/mL to about 250 mg/mL of a humanized anti-C1s antibody; (b) about 50 mM to about 200 mM arginine hydrochloride; (c) about 1 mM to about 50 mM histidine; (d) about 1% to about 10% (w/v) sorbitol; (e) about 1 μM to about 50 μM DTPA; and (f) about 0.01% to about 0.1% (w/v) polysorbate 80 (PS80), and wherein the composition has a pH of about 6 to about 7. In some embodiments, the arginine hydrocholoride is L-arginine hydrochloride.

In some embodiments, the disclosure provides a composition comprising (a) about 50 mg/mL to about 250 mg/mL of a humanized anti-C1s antibody; (b) about 50 mM to about 200 mM arginine hydrochloride; (c) about 1 mM to about 50 mM histidine; (d) about 1% to about 10% (w/v) trehalose; (e) about 1 μM to about 50 μM DTPA; and (f) about 0.01% to about 0.1% (w/v) polysorbate 80 (PS80), and wherein the composition has a pH of about 6 to about 7. In some embodiments, the arginine hydrocholoride is L-arginine hydrochloride.

In some embodiments, the disclosure provides a composition comprising (a) about 50 mg/mL to about 250 mg/mL of a humanized anti-C1s antibody; (b) about 50 mM to about 200 mM arginine hydrochloride; (c) about 1 mM to about 50 mM histidine; (d) about 1% to about 10% (w/v) sucrose; (e) about 1 μM to about 50 μM EDTA; and (f) about 0.01% to about 0.1% (w/v) polysorbate 80 (PS80), and wherein the composition has a pH of about 6 to about 7. In some embodiments, the arginine hydrocholoride is L-arginine hydrochloride.

In some embodiments, the disclosure provides a composition comprising (a) about 50 mg/mL to about 250 mg/mL of a humanized anti-C1s antibody; (b) about 50 mM to about 200 mM arginine hydrochloride; (c) about 1 mM to about 50 mM histidine; (d) about 1% to about 10% (w/v) sucrose; (e) about 5 mM to about 10 mM methionine; and (f) about 0.01% to about 0.1% (w/v) polysorbate 80 (PS80), and wherein the composition has a pH of about 6 to about 7. In some embodiments, the arginine hydrocholoride is L-arginine hydrochloride.

In some embodiments, the disclosure provides a composition comprising (a) about 50 mg/mL to about 250 mg/mL of a humanized anti-C1s antibody; (b) about 50 mM to about 200 mM arginine hydrochloride; (c) about 1 mM to about 50 mM histidine; (d) about 1% to about 10% (w/v) sucrose; (e) about 1 μM to about 50 μM DTPA; and (f) about 0.01% to about 0.1% (w/v) poloxamer 188 (P188), and wherein the composition has a pH of about 6 to about 7. In some embodiments, the arginine hydrocholoride is L-arginine hydrochloride.

In some embodiments, the disclosure provides a composition comprising (a) about 150 mg/mL of the antibody; (b) about 150 mM arginine HCl; (c) about 10 mM histidine; (d) about 3% (w/v) sucrose; (e) about 10 μM DTPA; and (f) about 0.06% (w/v) PS80, and wherein the composition has a pH of about 6.5 to about 7.1, or about 6.8. In some embodiments, a composition comprises (a) about 150 mg/mL of the antibody; (b) about 150 mM arginine HCl; (c) about 20 mM citrate; and (f) about 0.02% (w/v) PS80, and wherein the composition has a pH of about 6.5. In some embodiments, the arginine hydrocholoride is L-arginine hydrochloride.

A composition of the disclosure may comprise other excipients including, but not limited to, water for injection, diluents, solubilizing agents, soothing agents, additional buffers, inorganic or organic salts, antioxidants, or the like. In some embodiments, a composition of the disclosure comprises no other excipients, except those described above. Other pharmaceutically acceptable carriers or excipients such as those described in Remington's Pharmaceutical Sciences 16th edition, Osol, A. Ed. (1980) may be included in the formulation provided that they do not adversely affect the desired characteristics of the formulation. In some embodiments, a preservative may be added. In some embodiments, the composition is substantially free of preservatives. Cryoprotectants or lyoprotectants may be included in lyophilized formulations.

A composition of the disclosure can be in a liquid form, a lyophilized form wherein the lyophilized preparation is to be reconstituted with a sterile solution prior to administration, or a liquid form reconstituted from a lyophilized form. The standard procedure for reconstituting a lyophilized composition is to add back a volume of pure water (typically equivalent to the volume removed during lyophilization); however solutions comprising antibacterial agents can be used for the production of pharmaceutical compositions for parenteral administration; see also Chen (1992) Drug Dev Ind Pharm 18, 1311-54. In some embodiments, a composition of the disclosure is a liquid form. A liquid formulation may be ready for injection, or may be diluted prior to injection.

In some embodiments, the disclosure provides a stable liquid antibody formulation comprising a humanized anti-C1s antibody, arginine or a salt thereof, and one or more of: a buffer, a stabilizer or cryoprotectant, a chelator, and a surfactant.

In some embodiments, the disclosure provides a stable liquid antibody formulation comprising (a) about 50 mg/mL to about 250 mg/mL of a humanized anti-C1s antibody; (b) about 50 mM to about 200 mM arginine HCl; (c) about 1 mM to about 50 mM histidine; (d) about 1% to about 10% (w/v) sucrose; (e) about 1 μM to about 50 μM DTPA; and (f) about 0.01% to about 0.1% (w/v) polysorbate 80 (PS80), and wherein the composition has a pH of about 6 to about 7. In some embodiments, the arginine hydrocholoride is L-arginine hydrochloride.

In some embodiments, the disclosure provides a stable liquid antibody formulation comprising (a) about 150 mg/mL of the antibody; (b) about 150 mM arginine HCl; (c) about 10 mM histidine; (d) about 3% (w/v) sucrose; (e) about 10 μM DTPA; and (f) about 0.06% (w/v) PS80, and wherein the composition has a pH of about 6.5 to about 7.1, or about 6.8. In some embodiments, the arginine hydrocholoride is L-arginine hydrochloride.

The contemplated compositions are stable at 5° C. for at least about 1, 2, 3, 4, 5, 6, 7, 8, 9, 10, 11, or 12 months, or more. In some embodiments, the compositions are stable at 5° C. for at least about 12, 18, 24 or 30 months, or more. In some embodiments, they are stable at 5° C. for at least about 6 months or more. In some embodiments, they are stable at 5° C. for at least about 9 months. In some embodiments, they are stable at 5° C. for at least about 1 year or more, or more than about 2 years.

The compositions of the disclosure exhibit high levels of stability. The term “stable,” as used herein in reference to the compositions, means that the antibodies within the compositions retain an acceptable degree of structure and/or function and/or biological activity after storage for a defined amount of time. A composition may be stable even though the antibody contained therein does not maintain 100% of its structure and/or function and/or biological activity after storage for a defined amount of time. Under certain circumstances, maintenance of about 80%, about 85%, about 90%, about 95%, about 96%, about 97%, about 98% or about 99% of an antibody's structure and/or function and/or biological activity after storage for a defined amount of time may be regarded as “stable.”

In some embodiments, stability is measured by determining the percentage of native antibody remaining in the formulation after storage for a defined amount of time at a given temperature. The percentage of native antibody can be determined by, for example, size exclusion chromatography (e.g., size exclusion high performance liquid chromatography [SE-HPLC]) or any other method known in the art to characterize % monomer, % high molecular weight species, and % low molecular weight species. In some embodiments, stability is measured by determining thermal stability (e.g., by performing differential scanning calorimetry (DSC). In some embodiments, stability is measured by determining mechanical stability (e.g., by performing controlled agitation). In some embodiments, stability is measured by determining solution turbidity (e.g., by measuring optical density (OD) from 340 nm to 360 nm). In some embodiments, stability is determined by measuring sub-visible particles (e.g., by light obscuration using a liquid particle counter or by micro-flow imaging (MFI)). In some embodiments, stability is determined by measuring chemical degradation such as isomerization, oxidation, and deamidation using tryptic peptide mapping. In some embodiments, stability is determined by measuring solution opalescence using micro-nephelometry.

In some embodiments, stability may be assessed indirectly by measuring the PS80 concentration. In some embodiments, PS80 concentration is measured by high performance liquid chromatography with charged aerosol detector (HPLC-CAD).

Stability may also be assessed by measuring the biological activity and/or binding affinity of the antibody to its target. For example, a formulation of the present invention may be regarded as stable if, after storage at e.g., 5° C., 25° C., 45° C., etc. for a defined amount of time (e.g., 1 to 12 months), the humanized anti-C1s antibody contained within the composition binds to human C1s with an affinity that is at least 50%, 60%, 70%, 80%, 90%, 95%, or more of the binding affinity of the antibody prior to said storage.

The compositions of the disclosure inhibit antibody isomerization. In some embodiments, the isomerization is aspartic acid to isoaspartic acid. In some embodiments, the isomerization is at D32 of the light chain, located in CDR1, of the antibody. A certain level of isomerization may be acceptable as long as the function of the antibody is not compromised. Thus, a composition may have an acceptable level of isomerization even though the antibody contained therein does not maintain 100% of its structure and/or function and/or biological activity after storage for a defined amount of time. Under certain circumstances, maintenance of about 80%, about 85%, about 90%, about 95%, about 96%, about 97%, about 98% or about 99% of an antibody's structure and/or function and/or biological activity after storage for a defined amount of time may be regarded as acceptable levels of isomerization. Isomerization may be determined by any suitable method known in the art. In some embodiments, isomerization is determined by performing total peptide map analysis. In some embodiments, isomerization is measured by determining the percentage of isomerization in the formulation after storage for a defined amount of time at a given temperature. In some embodiments, the antibody has an isomerization rate of less than about 10%, less than about 9%, less than about 8%, or less than about 7.5% after 12 weeks of storage at 5° C. In some embodiments, the antibody has an isomerization rate of less than about 25%, less than about 24%, less than about 23%, less than about 21%, or less than about 20% after 12 weeks of storage at 25° C. In some embodiments, the antibody has an isomerization rate of less than about 40%, less than about 39%, less than about 38%, less than about 37%, less than about 36%, or less than about 35% after 12 weeks of storage at 40° C.

In some embodiments, the antibody has a lower isomerization rate (e.g., at D32 in the light chain CDR1) as compared to the antibody in a corresponding formulation without arginine or a salt thereof. In some embodiments, the antibody has an isomerization rate that is at least about 2% or at least about 3% per week at 40° C. In some embodiments, the antibody has an isomerization rate that is at least about 1-3% lower per month at 25° C. or at least about 0.4%, 0.5%, or 0.6% lower per month at 5° C., as compared to the antibody in a corresponding formulation without arginine or a salt thereof. In some embodiments, the antibody has an isomerization rate after 12 weeks of storage at 25° C. that is about 1%, 2%, 3%, 4%, 5%, 6%, 7%, 8%, 9%, or 10% lower as compared to the antibody in a corresponding formulation without arginine or a salt thereof.

### Routes of Administration

An antibody of the present disclosure can be administered to a subject using any available conventional methods and routes suitable for delivery of conventional drugs, including systemic or localized routes. In general, routes of administration contemplated by the disclosure include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, intrathecal, and intravenous routes, i.e., any route of administration other than through the alimentary canal. Parenteral administration can be carried to effect systemic or local delivery of a subject antibody. Where systemic delivery is desired, administration typically involves invasive or systemically absorbed topical or mucosal administration of pharmaceutical preparations.

Routes of administration can be combined, if desired, or adjusted depending upon the desired effect. A composition can be administered in a single dose or in multiple doses. In some embodiments, a composition of the disclosure is administered intravenously. In some embodiments, a composition of the disclosure is administered subcutaneously.

### Dosages and Dosage Forms

A suitable dosage can be determined by an attending physician or other qualified medical personnel, based on various clinical factors. As is well known in the medical arts, dosages for any one patient depend upon many factors, including the patient's size, body surface area, age, the particular compound to be administered, sex of the patient, time, and route of administration, general health, and other drugs being administered concurrently.

Those of skill will readily appreciate that dose levels and administration schedules can vary as a function of the specific antibody, the severity of the symptoms and the susceptibility of the subject to side effects. Preferred dosages and administration schedules for a given compound may be determined by those of skill in the art by a variety of means.

The disclosure also provides a pharmaceutical unit dosage form comprising a therapeutically effective amount of a composition of the disclosure for the treatment of one or more complement-mediated disease in a subject through administration of the dosage form to the subject. In some embodiments, the subject is a human. The term “pharmaceutical unit dosage form” refers to a physically discrete unit suitable as unitary dosages for the subjects to be treated, each unit containing a predetermined quantity of active compound calculated to produce the desired therapeutic/prophylactic effect.

The unit dosage form may be a container comprising the formulation. Suitable containers include, but are not limited to, sealed ampoules, vials (e.g., a glass vial), bottles, syringes, and test tubes. The containers may be formed from a variety of materials, such as glass or plastic, and may have a sterile access port (for example, the container may be a vial having a stopper that can be pierced by a hypodermic injection needle). In some embodiments, the container is a vial. In some embodiments, the container is a pre-filled syringe. Generally, the container should maintain the sterility and stability of the formulation.

In some embodiments, the composition is packaged in a vial (10R) that is made of type I glass, and closed with a stopper (flurotec coated chlorobutyl) sealed with flip-off caps with flange (aluminum). The vials are, in some embodiments, filled with 8 mL of the composition.

In some embodiments, the composition is packaged in a pre-filled syringe made of glass (OMPI) and closed with a rubber stopper (Novapure). The pre-filled syringes, are, in some embodiments, filled with 2 mL of the composition.

An exemplary drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

An exemplary sleeve-triggered auto-injector with manual needle insertion is described in International Publication WO2015/004052. Example audible end-of-dose feedback mechanisms are described in International Publications WO2016/193346 and WO2016/193348. An example needle-safety mechanism after using an auto-injector is described in International Publication WO2016/193352. An example needle sheath remover mechanism for a syringe auto-injector is described in International Publication WO2016/193353. An example support mechanism for supporting an axial position of a syringe is described in International Publication WO2016/193355.

### Kits and Articles of Manufacture

The present disclosure provides a kit or an article of manufacture comprising a composition of the disclosure. The kit or article of manufacture comprises a container comprising a composition of the disclosure. The kit or article of manufacture may further comprise one or more containers comprising pharmaceutically acceptable excipients, and include other materials desirable from a commercial and user standpoint, including filters, needles and syringes. Associated with the kits can be instructions customarily included in commercial packages of therapeutic, prophylactic or diagnostic products, that contain information about, for example, the indications, usage, dosage, manufacture, administration, contra-indications, and/or warnings concerning the use of such therapeutic, prophylactic or diagnostic products. The kit can also be associated with a label that can be any kind of data carrier (e.g., a leaflet, sticker, chip, print or bar code) comprising information. In certain embodiments, the instructions as listed above can be comprised in or on the label. The kit can further comprise a device for administration of the formulation, and particularly a device that contains the composition, i.e., a pre-filled device such as, but not limited to, a pre-filled syringe or a pre-filled autoinjector. The kit can also comprise a container comprising the composition, i.e., a pre-filled container, such as a pre-filled vial, cartouche, sachet, or ampoule.

### Complement-Mediated Diseases

The compositions of the present disclosure are useful for the treatment of complement-mediated diseases. Thus, in one aspect, the present disclosure provides methods of treating a complement-mediated disease. The method generally involves administering an effective amount of a composition of the disclosure to a subject in need thereof. In some cases, administration of a composition of the disclosure modulates the activity of complement C1s in a cell, a tissue, a fluid, or an organ of an individual, and treats the complement-mediated disease or disorder. The present disclosure provides methods of inhibiting activation of complement component C4 in an individual, the methods comprising administering to the subject an effective amount of a composition of the present disclosure. The present disclosure provides methods of inhibiting complement C1s activity in a subject, the methods comprising administering to the subject an effective amount of a composition of the present disclosure. The present disclosure provides methods of reducing the level of a complement component cleavage product in a subject (e.g., in a fluid, tissue, or organ in an individual), the methods comprising administering to the subject an effective amount of a composition of the present disclosure.

In some cases, an “effective amount” of a composition of the present disclosure is an amount that, when administered in one or more doses to a subject in need thereof, reduces the level of a complement component cleavage product in the subject (e.g., in a fluid, tissue, or organ in the individual). In some cases, an “effective amount” of a composition of the present disclosure, is an amount that, when administered in one or more doses to a subject in need thereof, reduces the level of a complement component cleavage product in the subject (e.g., in a fluid, tissue, or organ in the individual) by at least 10%, at least 20%, at least 30%, at least 40%, at least 50%, at least 60%, at least 65%, at least 70%, at least 75%, at least 80%, at least 85%, at least 90%, at least 95%, or 100%, compared to the level of the complement component cleavage product in the fluid, tissue, or organ in the absence of treatment with the composition, e.g., before treatment with the composition. In some embodiments, the complement component cleavage product is a C4 cleavage product (e.g., C4b). In some embodiments, the complement component cleavage product is a C2 cleavage product (e.g., C2a). In some embodiments, the complement component cleavage product is a C3 cleavage product.

In some cases, an “effective amount” of a composition of the present disclosure, is an amount that, when administered in one or more doses to a subject in need thereof, reduces the activity of the classical complement pathway in the subject (e.g., in a fluid, tissue, or organ in the individual). In some cases, an “effective amount” of a composition of the present disclosure, is an amount that, when administered in one or more doses to a subject in need thereof, reduces, within about 48 hours, within about 24 hours, within about 12 hours, within about 8 hours, or within about 4 hours of administration of the humanized anti-C1s antibody, the activity of the classical complement pathway in the subject (e.g., in a fluid, tissue, or organ in the individual), by at least 10%, at least 20%, at least 30%, at least 40%, at least 50%, at least 60%, at least 65%, at least 70%, at least 75%, at least 80%, at least 85%, at least 90%, at least 95%, or 100%, compared to the activity of the classical complement pathway in the fluid, tissue, or organ in the absence of treatment with the composition, e.g., before treatment with the composition. The level of activity of the classical complement pathway can be determined using any of a variety of methods. As one non-limiting example, the activity of the classical complement pathway can be determined ex vivo, e.g., by determining the level of activity of the classical complement pathway in a blood, serum, or plasma sample obtained from the individual. For example, the classical complement pathway in the blood, serum, or plasma sample can be activated ex vivo, and the amount of a complement component cleavage product (such as C5b-9) generated by such activation can be determined.

In some embodiments, an “effective amount” of a composition of the present disclosure, is an amount that, when administered in one or more doses to a subject in need thereof, maintains a reduction in the level of activity of the classical complement pathway in the subject (e.g., in a fluid, tissue, or organ in the individual) of at least 10%, at least 20%, at least 30%, at least 40%, at least 50%, at least 60%, at least 65%, at least 70%, at least 75%, at least 80%, at least 85%, at least 90%, at least 95%, or 100%, compared to the level of the activity of the classical complement pathway in the fluid, tissue, or organ in the absence of treatment with the composition, e.g., before treatment with the composition, where the reduction is maintained for a period of time of from about 4 hours to about 30 days (e.g., from 4 hours to 8 hours, from 8 hours to 24 hours, from 2 days to 4 days, from 4 days to 7 days, from 7 days to 14 days, from 14 days to 21 days, or from 21 days to 30 days).

In some embodiments, an “effective amount” of a composition of the present disclosure, is an amount that, when administered in one or more doses to a subject in need thereof, maintains a reduction in the level of a complement component cleavage product in the subject (e.g., in a fluid, tissue, or organ in the individual) of at least 10%, at least 20%, at least 30%, at least 40%, at least 50%, at least 60%, at least 65%, at least 70%, at least 75%, at least 80%, at least 85%, at least 90%, at least 95%, or 100%, compared to the level of the complement component cleavage product in the fluid, tissue, or organ in the absence of treatment with the composition, e.g., before treatment with the composition, where the reduction is maintained for a period of time of from about 4 hours to about 30 days (e.g., from 4 hours to 8 hours, from 8 hours to 24 hours, from 2 days to 4 days, from 4 days to 7 days, from 7 days to 14 days, from 14 days to 21 days, or from 21 days to 30 days).

In some cases, an “effective amount” of a composition of the present disclosure, is an amount that, when administered in one or more doses to a subject in need thereof, reduces production of C4b2a (i.e., complement C4b and C2a complex; also known as “C3 convertase”) in the subject (or in a fluid, tissue, or organ of the individual) by at least 10%, at least 20%, at least 30%, at least 40%, at least 50%, at least 60%, at least 65%, at least 70%, at least 75%, at least 80%, at least 85%, at least 90%, at least 95%, or 100%, compared to the amount of C4b2a produced in the subject, or the fluid, tissue, or organ, in the absence of treatment with the composition, e.g., before treatment with the composition.

The present disclosure provides a method to modulate complement activation. In some embodiments the method inhibits complement activation, for example to reduce production of C4b2a. In some embodiments, the present disclosure provides a method to modulate complement activation in an individual having a complement-mediated disease, the method comprising administering to the individual a composition of the present disclosure. In some embodiments such a method inhibits complement activation.

In some embodiments, a complement-mediated disease is characterized by the presence in a cell, a tissue, or a fluid of an elevated (higher than normal) amount of C1s or of an elevated level of complement C1s activity. For example, in some embodiments, a complement-mediated disease is characterized by the presence in brain tissue and/or cerebrospinal fluid of an elevated amount and/or an elevated activity of C1s. A “higher than normal” amount of C1s in a cell, a tissue, or a fluid indicates that the amount of C1s in the cell, tissue or fluid is higher than a normal, control level, e.g., higher than a normal, control level for an individual or population of individuals of the same age group. A “higher than normal” level of C1s activity in a cell, a tissue, or a fluid indicates that the proteolytic cleavage effected by C1s in the cell, tissue or fluid is higher than a normal, control level, e.g., higher than a normal, control level for an individual or population of individuals of the same age group. In some embodiments, an individual having a complement-mediated disease exhibits one or more additional symptoms of such a disease. It should be understood that the term “disease” encompasses “disorders.” The two terms may be used interchangeably. In some embodiments, a complement-mediated disease is a classical complement-mediated disease.

In some embodiments, a complement-mediated disease is characterized by the presence in a cell, a tissue, or a fluid of a lower than normal amount of C1s or of a lower level of complement C1s activity. For example, in some embodiments, a complement-mediated disease is characterized by the presence in brain tissue and/or cerebrospinal fluid of a lower amount and/or a lower activity of C1s. A “lower than normal” amount of C1s in a cell, a tissue, or a fluid indicates that the amount of C1s in the cell, tissue or fluid is lower than a normal, control level, e.g., lower than a normal, control level for an individual or population of individuals of the same age group. A “lower than normal” level of C1s activity in a cell, a tissue, or a fluid indicates that the proteolytic cleavage effected by C1s in the cell, tissue or fluid is lower than a normal, control level, e.g., lower than a normal, control level for an individual or population of individuals of the same age group. In some embodiments, an individual having a complement-mediated disease exhibits one or more additional symptoms of such a disease.

A complement-mediated disease is a disease in which the amount or activity of complement C1s is such as to cause disease in an individual. In some embodiments, the complement-mediated disease is selected from the group consisting of alloimmune disease, autoimmune disease, cancer, hematological disease, infectious disease, inflammatory disease, ischemia-reperfusion injury, neurodegenerative disease, neurodegenerative disorder, ocular disease, renal disease, transplant rejection, vascular disease, and vasculitis disease. In some embodiments, the complement-mediated disease is an autoimmune disease. In some embodiments, the complement-mediated disease is an alloimmune disease. In some embodiments, the complement-mediated disease is cancer. In some embodiments, the complement-mediated disease is an infectious disease. In some embodiments, the complement-mediated disease is an inflammatory disease. In some embodiments, the complement-mediated disease is a hematological disease. In some embodiments, the complement-mediated disease is an ischemia-reperfusion injury. In some embodiments, the complement-mediated disease is ocular disease. In some embodiments, the complement-mediated disease is a renal disease. In some embodiments, the complement-mediated disease is transplant rejection. In some embodiments, the complement-mediated disease is antibody-mediated transplant rejection. In some embodiments, the complement-mediated disease is a vascular disease. In some embodiments, the complement-mediated disease is a vasculitis disorder. In some embodiments, the complement-mediated disease is a neurodegenerative disease.

Examples of a complement-mediated disease or disorder include, but are not limited to, age-related macular degeneration, Alzheimer's disease, amyotrophic lateral sclerosis, anaphylaxis, antibody mediated rejection (AMR), argyrophilic grain dementia, arthritis (e.g., rheumatoid arthritis), asthma, atherosclerosis, atypical hemolytic uremic syndrome, autoimmune diseases (including, e.g., autoimmune hemolytic anemia (AIHA); warm AIHA; mixed AIHA; etc.), Barraquer-Simons syndrome, Behçet's disease, British type amyloid angiopathy, bullous pemphigoid, Buerger's disease, C1q nephropathy, cancer, catastrophic antiphospholipid syndrome, cerebral amyloid angiopathy, chronic inflammatory demyelinating polyradiculoneuropathy (CIDP), cold agglutinin disease (CAD), corticobasal degeneration, Creutzfeldt-Jakob disease, Crohn's disease, cryoglobulinemic vasculitis, dementia pugilistica, dementia with Lewy Bodies (DLB), diffuse neurofibrillary tangles with calcification, Discoid lupus erythematosus, Down's syndrome, Evan's syndrome, focal segmental glomerulosclerosis, formal thought disorder, frontotemporal dementia (FTD), frontotemporal dementia with parkinsonism linked to chromosome 17, frontotemporal lobar degeneration, Gerstmann-Straussler-Scheinker disease, Guillain-Barré syndrome, Hallervorden-Spatz disease, hemolytic-uremic syndrome, hereditary angioedema, hypophosphastasis, idiopathic pneumonia syndrome, immune complex diseases, inclusion body myositis, infectious disease (e.g., disease caused by bacterial (e.g., Neisseria meningitidis or Streptococcus) viral (e.g., human immunodeficiency virus (HIV)), or other infectious agents), inflammatory disease, ischemia/reperfusion injury, mild cognitive impairment, immunothrombocytopenic purpura (ITP), molybdenum cofactor deficiency (MoCD) type A, membranoproliferative glomerulonephritis (MPGN) I, membranoproliferative glomerulonephritis (MPGN) II (dense deposit disease), membranous nephritis, multi-infarct dementia, lupus (e.g., systemic lupus erythematosus (SLE)), glomerulonephritis, Kawasaki disease, multifocal motor neuropathy, multiple sclerosis, multiple system atrophy, myasthenia gravis, myocardial infarction, myotonic dystrophy, neuromyelitis optica, Niemann-Pick disease type C, non-Guamanian motor neuron disease with neurofibrillary tangles, Parkinson's disease, Parkinson's disease with dementia, paroxysmal nocturnal hemoglobinuria, Pemphigus vulgaris, Pick's disease, postencephalitic parkinsonism, polymyositis, prion protein cerebral amyloid angiopathy, progressive subcortical gliosis, progressive supranuclear palsy, psoriasis, sepsis, Shiga-toxin E coli (STEC)-HuS, spinal muscular atrophy, stroke, subacute sclerosing panencephalitis, Tangle only dementia, transplant rejection, vasculitis (e.g., ANCA associated vasculitis), Wegner's granulomatosis, sickle cell disease, cryoglobulinemia, mixed cryoglobulinemia, essential mixed cryoglobulinemia, Type II mixed cryoglobulinemia, Type III mixed cryoglobulinemia, nephritis, drug-induced thrombocytopenia, lupus nephritis, Epidermolysis bullosa acquisita, delayed hemolytic transfusion reaction, hypocomplementemic urticarial vasculitis syndrome, pseudophakic bullous keratopathy, and platelet refractoriness.

In some embodiments, the complement-mediated disease is CAD. In some embodiments, the complement-mediated disease is ITP. In some embodiments, the complement-mediated disease is CIDP. In some embodiments, the complement-mediated disease is AMR.

In some embodiments, a composition of the present disclosure prevents or delays the onset of at least one symptom of a complement-mediated disease in a subject. In some embodiments, a composition of the present disclosure reduces or eliminates at least one symptom of a complement-mediated disease in a subject. Examples of symptoms include, but are not limited to, symptoms associated with autoimmune disease, cancer, hematological disease, infectious disease, inflammatory disease, ischemia-reperfusion injury, neurodegenerative disease, neurodegenerative disorder, renal disease, transplant rejection, ocular disease, vascular disease, or a vasculitis disorder. The symptom can be a neurological symptom, for example, impaired cognitive function, memory impairment, loss of motor function, etc. The symptom can also be the activity of C1s protein in a cell, tissue, or fluid of an individual. The symptom can also be the extent of complement activation in a cell, tissue, or fluid of an individual.

By “treatment” is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as a complement-mediated disease. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts (wherein the term “host” is used interchangeably herein with the terms “subject,” “individual,” and “patient”) are treatable according to the subject methods. Generally such hosts are “mammals” or “mammalian,” where these terms are used broadly to describe organisms which are within the class mammalia, including the orders carnivore (e.g., cats), herbivores (e.g., cattle, horses, and sheep), omnivores (e.g., dogs, goats, and pigs), rodentia (e.g., mice, guinea pigs, and rats), and primates (e.g., humans, chimpanzees, and monkeys). In some embodiments, the host is an individual that has a complement system, such as a mammal, fish, or invertebrate. In some embodiments, the host is a complement system-containing mammal, fish, or invertebrate companion animal, agricultural animal, work animal, zoo animal, or lab animal. In some embodiments, the host is human.

### EXAMPLES

### Example 1. Design of Formulation Development Studies

SAR445088 is a humanized immunoglobulin G (IgG) subclass 4 (IgG4), monoclonal antibody (mAb), produced in Chinese hamster ovary (CHO) cells, that binds to and inhibits the activated form of C1 s within the classical pathway (CP). The mechanism of SAR445088 is specific to the CP, leaving the lectin and the alternative pathways functionally intact. The LC CDR1, LC CDR2, LC CDR3, HC CDR1, HC CDR2, HC CDR3, VH, VL, and heavy and light chain sequences of the SAR445088 are described elsewhere in the application as SEQ ID NOs. 1-10. The sequences are also described in U.S. Pat. Nos. 9,512,233, 10,729,767, and U.S. Patent Application Publication No. US 2020/0048332, each of which is herein incorporated by reference in its entirety.

Early-stage development studies have shown that this antibody has a higher propensity to self-associate (kD −22.7 mL/g in 10 mM histidine, pH 6.0 and −12.4 mL/g in 10 mM histidine, pH 6.0+150 mM NaCl) and has a tendency for isomerization at an aspartic acid in the light chain CDR1 (LC D32 isomerization). Formulation development studies were conducted to assess the feasibility and liabilities associated with an optimized, histidine-based liquid drug product (DP). A set of comprehensive studies was performed to understand the impact of formulation composition and process conditions on the biophysical and chemical stability of SAR445088. The impacts of buffer species and pH, polysorbate 80 concentrations, and metal chelators were all critically assessed for optimized development of liquid SAR445088 DP. The specific assessments and their purpose were as follows:

### Managing D32 Isomerization

To identify the edge of failure for prevention of D32 isomerization and to assess the impact of pH and stabilizer on D32 isomerization.

### Polysorbate 80 Screen

To assess the optimal concentrations of polysorbate 80 (PS80) needed to stabilize SAR445088 liquid formulations upon agitation-induced stress and exposure to light. In addition, the optimal PS80 concentration in the liquid DP was determined by testing the ability of various PS80 concentrations to mitigate turbidity increases and/or sub-visible particle formation in SAR445088 samples upon a 50-fold dilution to 3 mg/mL with 0.9% normal saline.

### Metal Spiking and Chelator Comparison Study

To examine the impact of transition metals, which may leach into drug substances (DSs) during manufacturing, on the chemical and physical stability of SAR445088 and PS80. Chelators EDTA and DTPA were also evaluated for their ability to protect the protein from degradation during a set of worst-case stability experiments.

pH/Buffer Screen and DP Stability

To evaluate the effects of buffer identity and pH on the physical and chemical stability of SAR445088 liquid formulations during refrigerated storage, ambient storage, and accelerated storage conditions. Histidine and phosphate buffer systems were chosen for this study. The proposed target formulation matrix was tested for its ability to stabilize high concentration DP solutions during frozen and liquid storage. Effect of addition of 3% sucrose on DP stability was also evaluated.

### DP Robustness Studies

To ensure the final composition of the proposed target formulation is sufficiently robust to withstand agitation-induced stresses, freeze-thawing, and light exposure with no loss of formulation stability during storage or manufacturing.

### Example 2. Materials and Methods

### Materials

Drug substance. SAR445088 formulated drug substance (FDS) was prepared at >150 mg/mL concentration. For a given study performed during formulation development, a specific lot and batch of FDS was used. After formulating the FDS to the target protein and excipient concentrations, formulations were then 0.22 μm-filtered under laminar flow before aseptic filling to prepare the liquid drug product (DP).

Excipients. All excipients used during formulation development studies were ACS, United States Pharmacopeia (USP) and European Pharmacopeia (Eur. Ph.) grade raw materials. The excipients used in the studies included L-Histidine, L-Histidine monohydrochloride, L-Arginine monohydrochloride, sucrose, PS80, sodium phosphate monobasic, sodium phosphate dibasic7-hydrate, EDTA, and DTPA. All excipients were obtained from J. T. Baker with the exception of DTPA, which was obtained from Sigma Aldrich.

### Analytical Methods

pH. The pH of buffers and formulated antibody solutions was measured using a Mettler-Toledo pH probe and meter. The results were considered comparable when the difference between repeated measurements was within 0.1 pH units.

Lunatic for protein concentration. Total protein concentration was determined by measuring the UV absorbance at 280 nm on microfluidic chips on a Big Lunatic spectrometer (Unchained Labs). Measurements were performed in duplicate on 2 to 5 μL volumes of sample (ε1%: 14.6 (g/100 mL)−1 cm−1).

UV plate reader for turbidity and optical density. Sample turbidity was quantified by measuring optical density (OD) from 340 nm to 360 nm on a SpectraMax i3 Microplate Reader (Molecular Devices). For each sample, 200 μL was loaded onto a UV-Vis transparent 96 well plate (Corning). The OD was determined as the average of absorbance values at 340 nm, 345 nm, 350 nm, 355 nm, and 360 nm.

Size-exclusion HPLC for high molecular weight (HMW) species. Aggregation analysis was performed by size exclusion chromatography (SEC). Samples were resolved on a 1260 series HPLC (Agilent, Santa Clara, CA) equipped with a TSK-GEL G3000SWXL (Tosoh Bioscience, Tokyo, Japan) analytical column and matching guard column in 20 mM sodium phosphate, 300 mM sodium chloride, pH 6.5±0.1 at a flowrate of 0.5 mL/min for 30 minutes. Two injections were performed for each sample. Detection was carried out by UV absorbance at 280 nm and the chromatographic peaks were integrated to determine the relative percentage of each eluted species.

Size-exclusion ultra-high pressure liquid chromatography (UPLC) for HMW and low molecular weight (LMW) species. Aggregation analysis was performed by size exclusion chromatography (SEC). Samples were resolved on a Waters UPLC (Waters Corporation, Milford, MA) equipped with a Acquity BEH200 UPLC (Waters Corporation, Milford, MA) analytical column in 50 mM sodium phosphate, 300 mM sodium chloride, pH 7.0 with a flowrate of 0.3 mL/min for 20 minutes. Three injections were performed for each sample. Detection was carried out by UV absorbance at 280 nm and the chromatographic peaks were integrated to determine the relative percentage of each eluted species.

Subvisible particles. Sub-visible particles were also measured by light obscuration on a High accuracy liquid particle counter (HIAC) Model 9703+ (Beckman Coulter). The counter was flushed with 0.22 μm-filtered and degassed MilliQ water until particle counts were ≤1 particle/mL at 10 μm, 2 μm, 10 μm and 25 μm standards were measured to ensure accurate particle counts, then the counter was extensively washed to remove background signal. Using a 1 mL protocol, samples were analyzed using four separate injections of 0.2 mL volumes of sample. The first sample measurement was disregarded and the following three were averaged.

cIEF for charge variants. Protein charge heterogeneity was measured by capillary isoelectric focusing (cIEF) using a Maurice instrument (Protein Simple) with detection via UV absorbance at 280 nm. The samples (1 mL) and the standard solutions were first diluted to 15 mg/mL in formulation buffer, then diluted up to 5 mg/mL with water. Onboard mixing was used to mix samples with MasterMix before analyses. The isoelectric focusing of the samples consisted of a pre-focusing period of 3 minutes at 1500 V followed by a focusing period of over 11 minutes at 3000 V. Results were considered comparable when the difference between samples was equal to or less than 10%.

PS80 determination by charged aerosol detection (CAD). PS80 is a non-ionic surfactant, consisting mainly of a hydrophilic sorbitan polyoxyethylene group linked by an ester to a fatty acid. The separation of PS80 from other sample components, such as protein and formulation constituents, was accomplished by mixed-mode HPLC using an Oasis MAX column (2.1×20 mm, 30 μm particle size, part #186002052) (Waters) and charged aerosol detection (CAD) (Agilent Technologies, CA). The column resin contains a hydrophobic polymer backbone supporting positively charged quaternary amines. As a result, this column provides a combination of reversed-phase and ion exchange capabilities. This allows retention of hydrophobic compounds, such as PS80, and generates electrostatic repulsion to positive charges on proteins/excipients. Samples were eluted using a mobile phase gradient of 2% formic acid in water (A) and 2% formic acid in isopropyl alcohol (B). Each sample was injected and passed through the column for 8 minutes with CAD within the 2.5 to 8 minute detection window.

Quantification of post-translational modifications (PTMs) by LC-MS. Protein samples were diluted to 1 mg/mL, then reduced, alkylated, and digested with a mixture of trypsin and Lys-C using a fully automated method on a STAR liquid handling system (Hamilton). The peptide mapping analysis was performed on a Q Exactive HF LC-MS system. The acquired LC-MS and LC-MS/MS data were processed using BioPharma Finder 4.1 for identification and relative quantitation of modifications.

Potency. C1s was directly immobilized on the surface of Biacore SPR sensor chips. Calibration curves of C1s binding by antibody were generated using the original samples of formulated antibody without buffer exchange. All samples were tested after 1000-fold dilution in running buffer. All samples were tested in triplicate by three different dilution schemes (100×10, 50×20 and 25×40). Control samples were the original samples diluted to 1 μg/mL. Control samples were run every 6 samples.

### Example 3. Management of D32 Isomerization

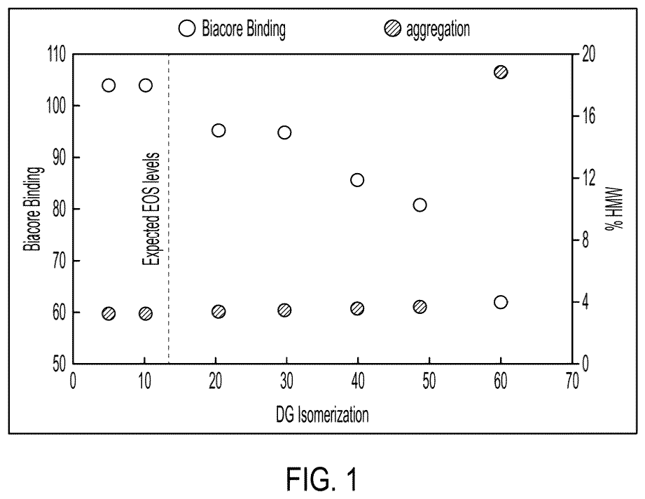

D32 isomerization in light chain (LC) CDR1 region of SAR445088 was identified as a major chemical degradation mechanism which directly affected the Biacore binding of the SAR445088 molecule. As seen in FIG. 1, Biacore binding was inversely affected by D32 isomerization in the CDR1. Biacore binding strongly decreased beyond an isomerization rate of 40%. Based on a benchmarking exercise, expected end of shelf-life (EOS) level of D32 isomerization was estimated to be around 10-15%.

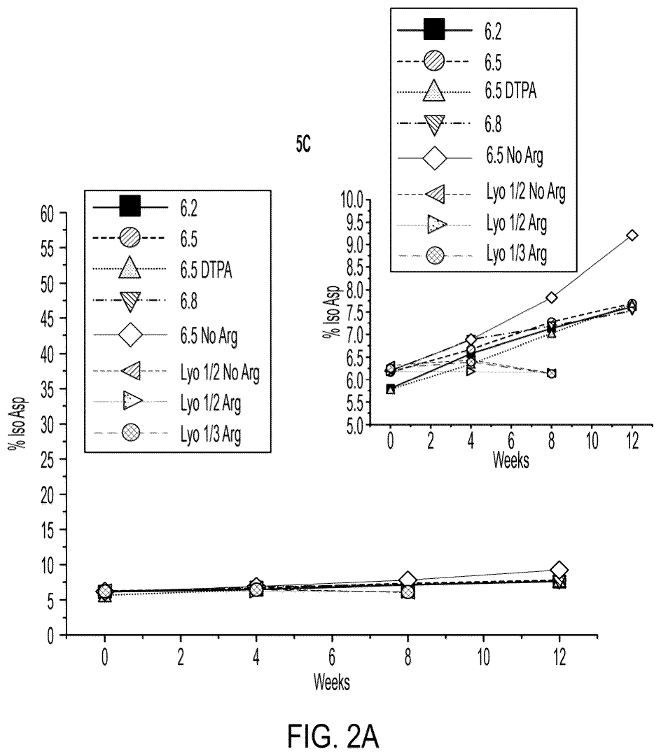

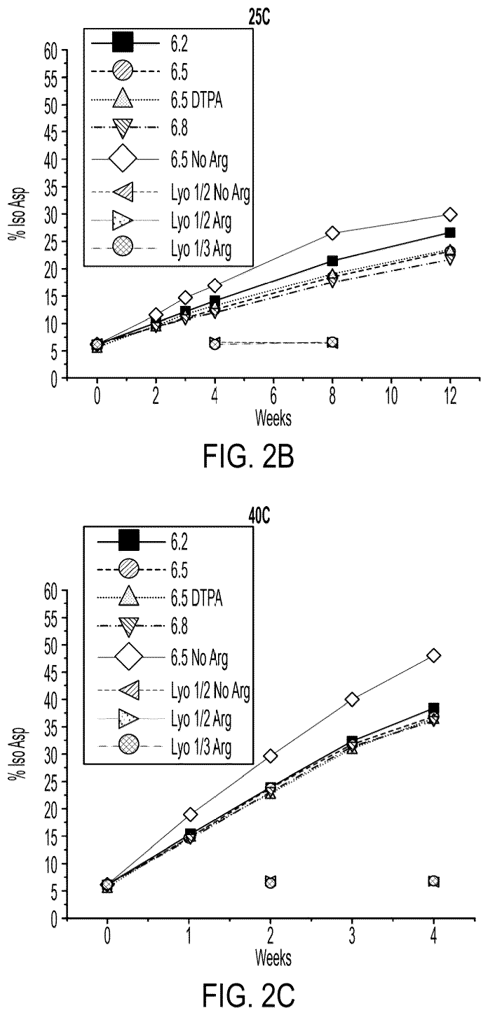

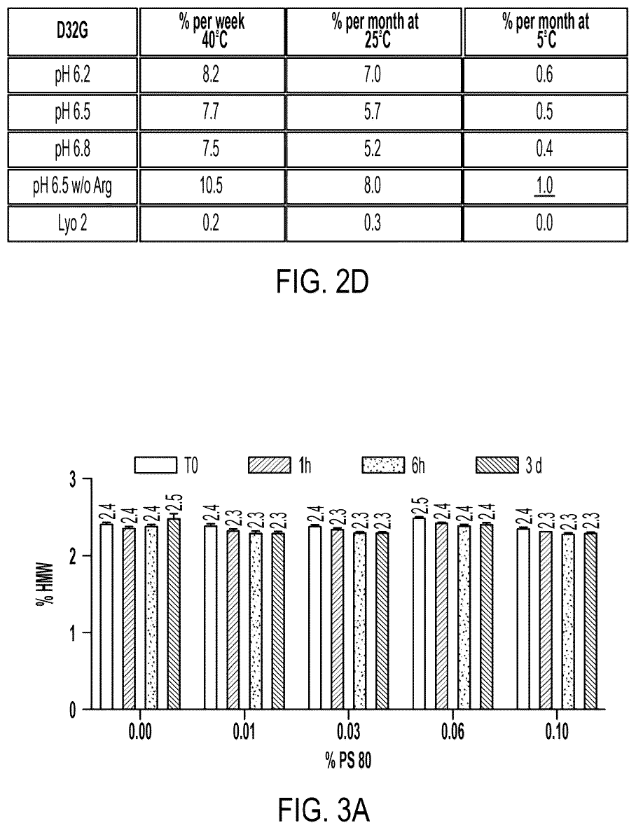

Next, a systematic study was performed to evaluate the effect of pH and presence of stabilizer such as arginine hydrochloride on SAR445088's isomerization rate. As seen in FIGS. 2A-2D, at all the tested temperatures, D32 isomerization rate decreased as the pH increased from 6.2 to 6.8. Further, addition of arginine hydrochloride as a stabilizer further helped reduce the isomerization rate compared to the counterpart without arginine hydrochloride. As expected, lyophilized formulation showed negligible isomerization at all the tested temperatures. Based on these results, a higher pH (pH 6.8) and inclusion of arginine hydrochloride appeared beneficial for further antibody formulation development. Liquid formulations with a high pH can permit deamidation-based degradation, and a preferred SAR445088 formulation has an optimum pH and stabilizer concentration to minimize both isomerization and deamidation.

### Example 4. PS80 Justification Studies

An optimal amount of PS80 in an SAR445088 formulation was determined by observing the resistance of a test formulation (150 mg/mL SAR445088, 150 mM Arginine HCl, 3% sucrose, 10 μM EDTA, 10 mM Histidine, pH 7.0) containing 0-0.1% PS80 to various stresses described in Table 2. The aim of the study was to determine a suitable amount of PS80 for reducing the effects of shear during manufacturing, shipping, and handling, using the study conditions described below.

TABLE 2
Study conditions for PS80 justification.

| STUDY | METHODOLOGY |
|---|---|
| Orbital shaking | Samples were put on an orbital shaker with a speed |
| | of 300 rpm for 1 h, 6 h and 3 days |
| Wrist action shaking | Samples were put on a wrist action shaker for 1 h, 3 h |
| | and 6 h |
| Wrist action shaking + | Samples spiked with silicone oil were put on a wrist |
| silicone spiking | action shaker for 1 h, 3 h and 6 h |
| IV Bag Dilution | Samples diluted 50-fold with 0.9% normal saline to |
| | 3 mg/mL antibody were left in IV bags under |
| | ambient temperature and ambient light for 24 hours |
| Photostability | Samples were kept on a shelf at room temperature |
| | and ambient light for 6 h and 3 days |

### Orbital Shaking Study

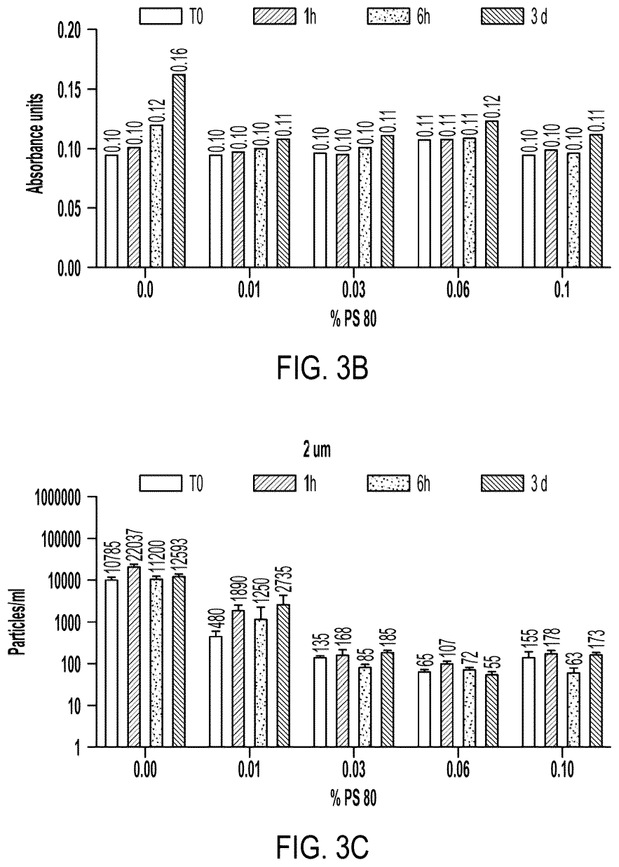

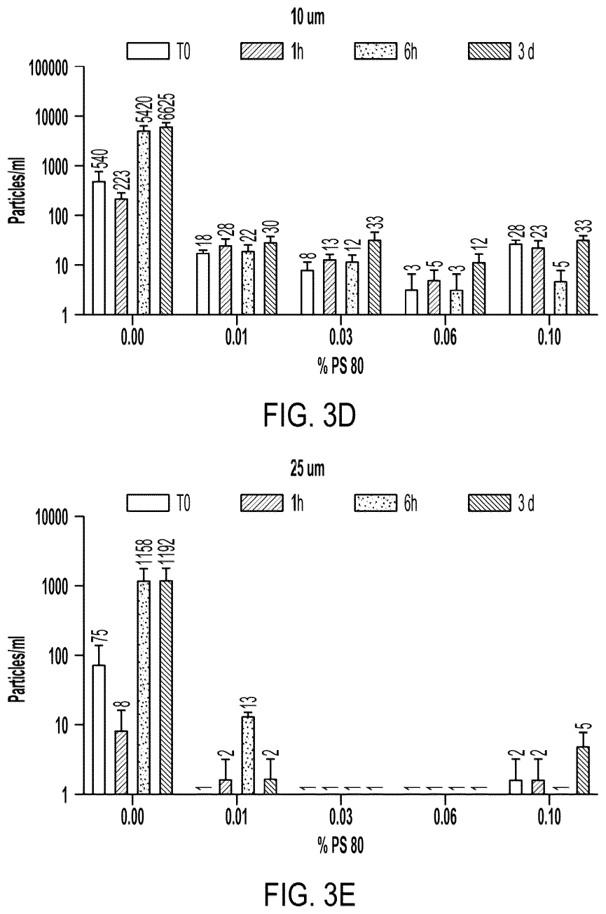

As seen from FIG. 3, the presence of PS80 did not impact the formation of protein HMW species upon orbital shaking (FIG. 3A). Samples without PS80 showed an increase in optical density/turbidity upon orbital shaking over time. However, samples containing as little as 0.01% PS80 showed greater stability (FIG. 3B). Lastly, samples without PS80 showed an increase in 2 μm, 10 μm and 25 μm sub-visible particles upon orbital shaking over time. However, samples containing >0.01% PS80 showed little to no increase in sub-visible particle formation (FIG. 3C-E). Therefore, a concentration of >0.01% PS80 can control degradation of SAR445088 degradation from shaking.

### Wrist Action Shaking

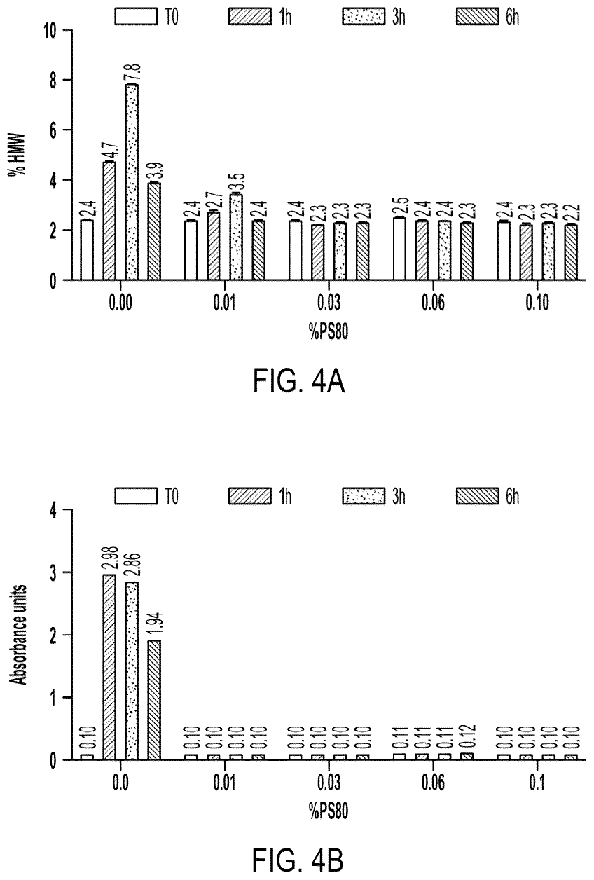

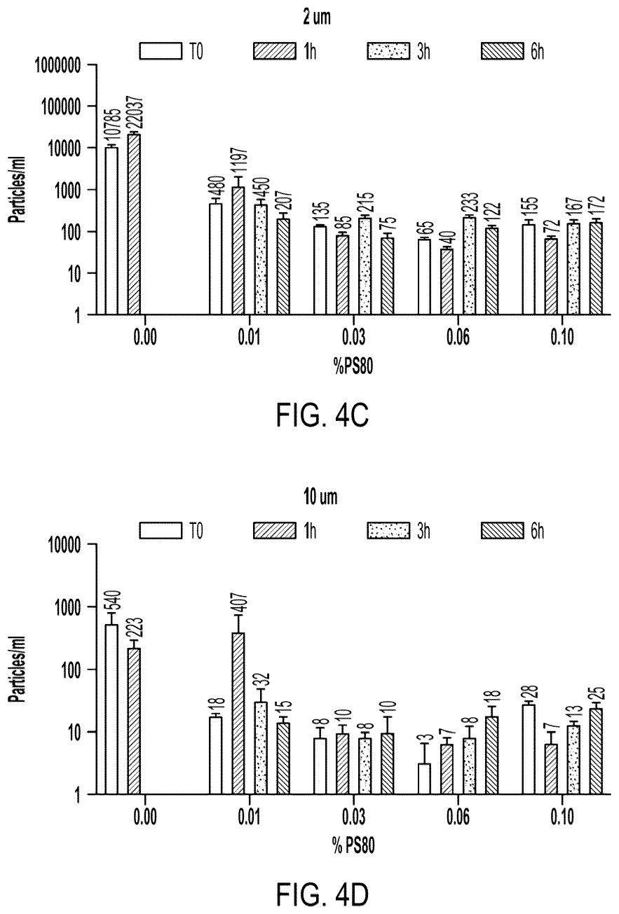

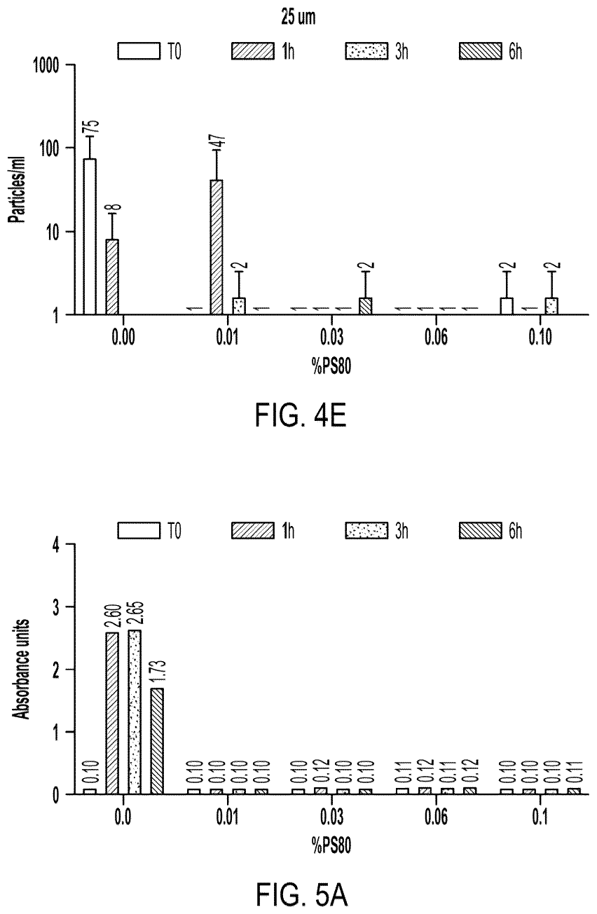

As seen in FIG. 4, samples without PS80 showed the highest propensity of protein to form aggregates (HMW species) upon shaking. Samples with >0.03% PS80 showed good resistance to HMW species formation (FIG. 4A). Samples without PS80 showed increased turbidity as early as 1 h after wrist action shaking. 0.01% PS80 was sufficient to prevent the increase in turbidity (FIG. 4B). Samples without PS80 showed the highest number of 2 μm, 10 μm and 25 μm sub-visible particles, as early as 1 h after shaking. Thereafter, an insoluble whitish precipitate appeared, and the samples without PS80 were not further assessed for sub-visible particles. Samples with >0.03% PS80 showed resistance to sub-visible particle formation (FIGS. 4C-E).

### Wrist Action Shaking+Silicone Spiking

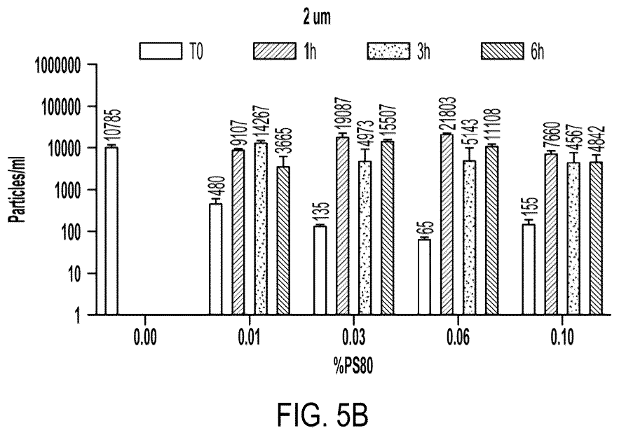

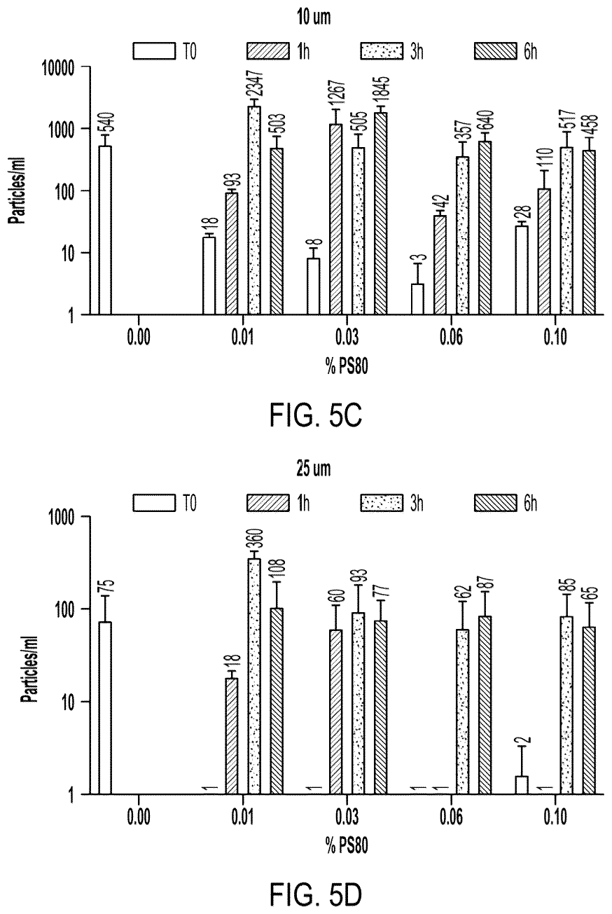

As seen in FIG. 5, samples without PS80 showed increase in turbidity, as early as 1 h, upon wrist action shaking. 0.01% PS80 was sufficient to prevent the increase in turbidity. An insoluble whitish precipitate formed in samples without PS80, and these samples were not assessed for particles after T0 (FIG. 5A). Samples with >0.01% PS80 had a higher number of 2 μm sub-visible particles due to presence of silicone oil. Samples with >0.03% PS80 showed resistance to formation of 10 μm and 25 μm particles upon wrist action shaking in the presence of silicone (FIGS. 5B-D). SEC analysis was not performed to prevent the column from being damaged by the silicone oil.

### IV Bag Dilution Study

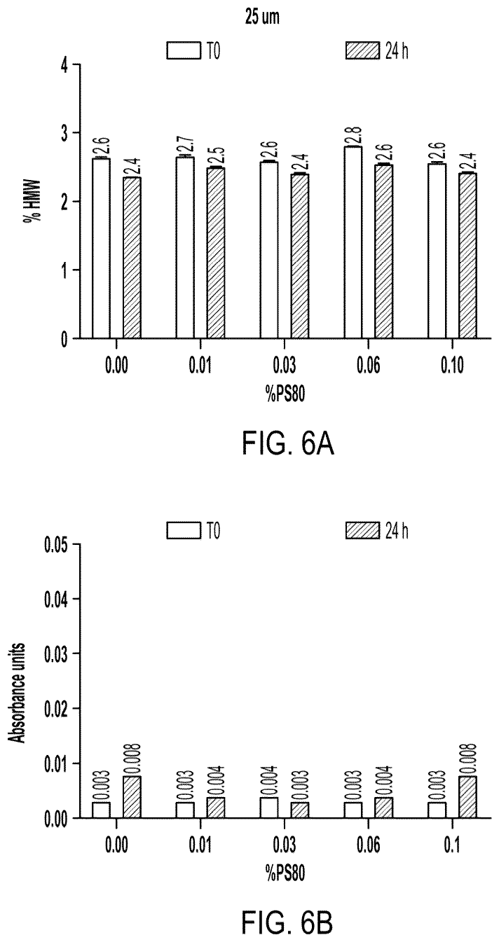

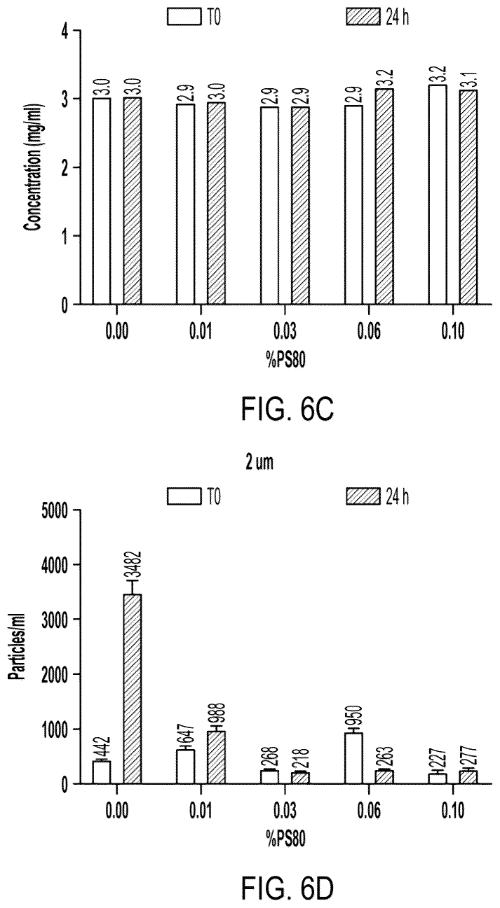

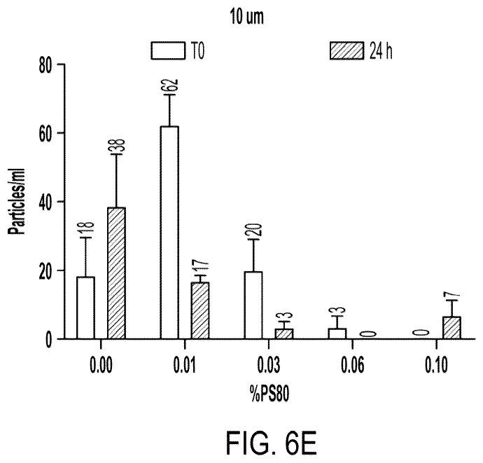

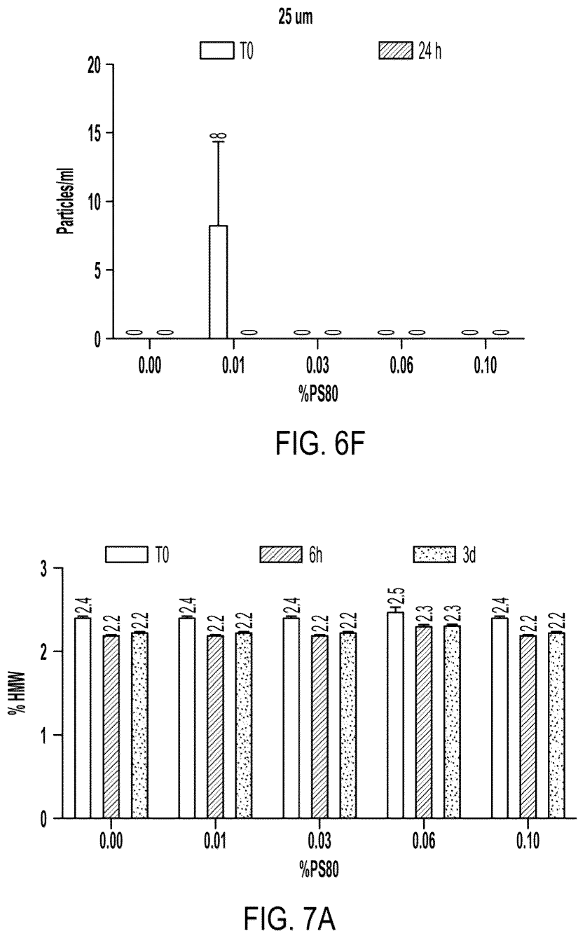

As seen in FIG. 6, PS80 showed no impact on protein aggregate formation in samples upon dilution and holding in IV bags (FIG. 6A). All PS80 concentrations had minimal impact on turbidity over 24 hours in the test IV bags (FIG. 6B). All IV bags had a target protein concentration of 3 mg/mL (FIG. 6C). Samples containing <0.01% PS80 showed an increase in 2 μm and 10 μm sub-visible particles over 24 h in IV bags. Samples containing >0.03% PS80 showed resistance to sub-visible particle formation (FIGS. 6D-F).

### Short-Term Photostability Study

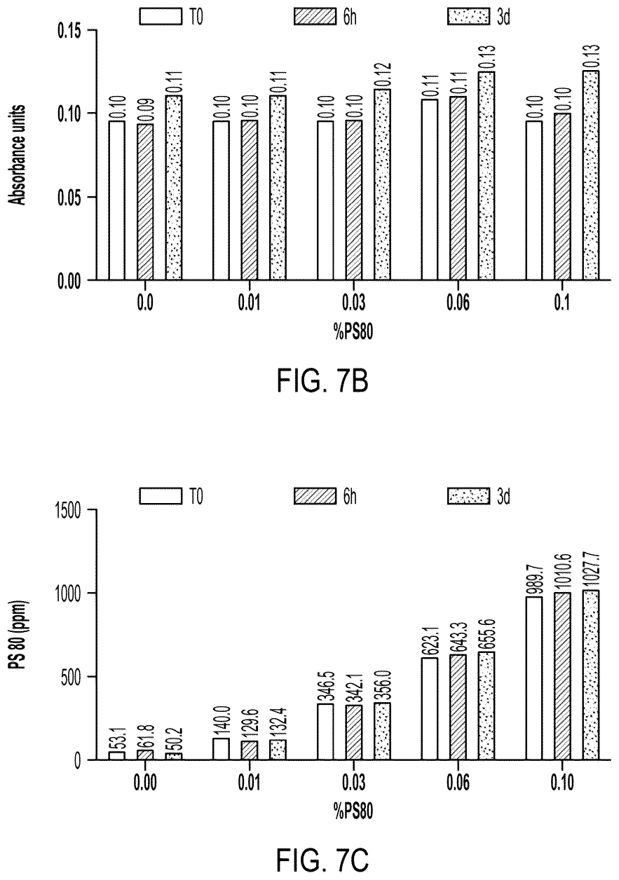

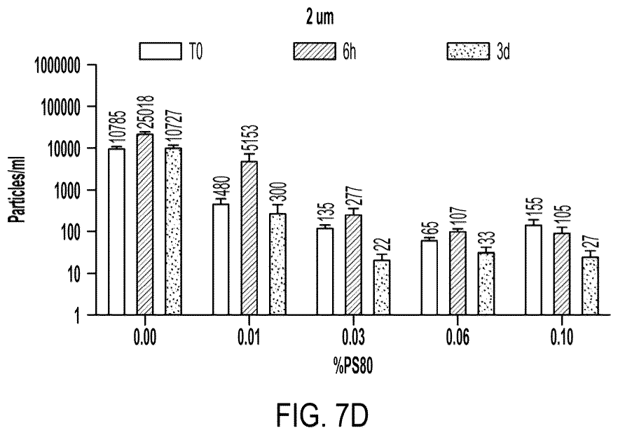

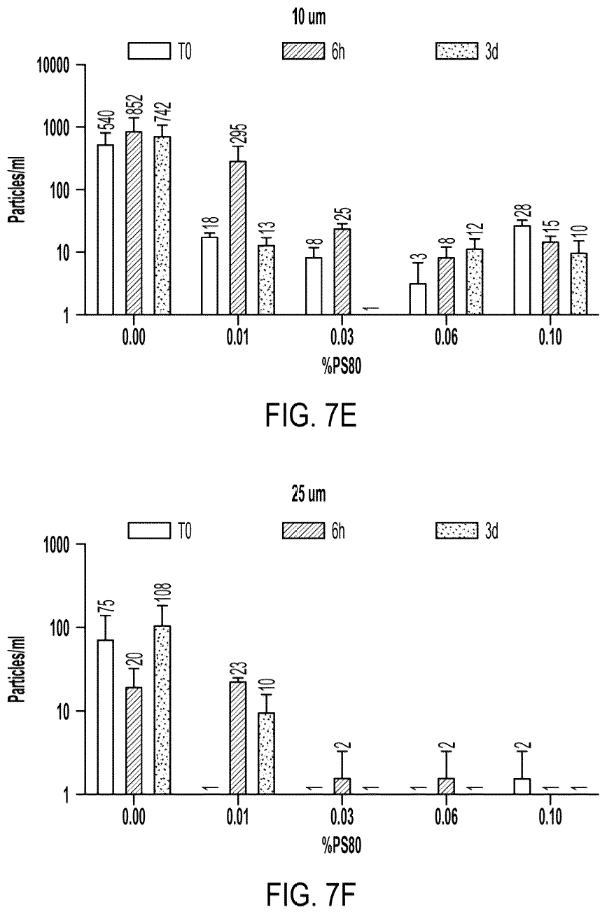

As seen in FIG. 7, PS80 did not impact the ability of the protein to form HMW species upon exposure to ambient light and all tested levels of PS80 had a similar effect on HMW species formation (FIG. 7A). An increase in turbidity was apparent in all samples upon exposure to ambient light for 3 days (FIG. 7B). No PS80 degradation was observed over the course of the entire study for any of the tested conditions (FIG. 7C). Samples containing <0.01% PS80 showed an increase in 2 μm, 10 μm and 25 μm particles over time. Samples with >0.01% PS80 showed resistance to particle formation (FIGS. 7D-F).

### Summary

Considering all mechanical, physical and chemical stresses that the formulation is expected to encounter during its life cycle and that are reflected in the stress tests performed in this study, at least 0.03% PS80 appeared necessary to provide minimally acceptable protection against stress-induced HMW species and sub-visible particle formation. However, 0.06% PS80 (600 ppm) provided more sufficient protection to the antibody formulation against all the tested stresses.

### Example 5. Chelator Justification Studies

The identity and concentration of an appropriate chelator for SAR445088 formulations to prevent PS80 oxidation was determined. Different FDS batches containing 150 mg/mL SAR445088, 150 mM Arginine HCl, 3% sucrose, 0.06% PS80, 10 mM Histidine, pH 7.0 were spiked with chelators (EDTA or DTPA) and metal ions (iron (Fe), copper (Cu), and tungsten (W) and subjected to refrigerated, ambient-temperature, and accelerated (40° C.) stability testing as described in Table 3. The two chelators were selected based on use in approved biologics products. The metal ions were selected based on metals found in water used for manufacturing, possible stainless steel vessel or part contact, and tungsten for needle formation of the glass prefilled syringes.

TABLE 3
Chelator and metal ion spiking plan

| | EDTA | DTPA | | Temperature |
|---|---|---|---|---|
| FDS batch | (μM) | (μM) | Metal ions | (° C.) |
| 1 | 10, 50 | 10, 50 | 50 ppb Fe, Cu, W | 25, 40 |
| 2 | 10, 50 | 10, 50 | 50 ppb Fe, Cu, W | 25, 40 |
| 3 | 10, 25, 50 | 10, 25, 50 | — | 5, 25, 40 |
| 4 | 10, 25, 50 | 10, 25, 50 | — | 5, 25, 40 |

### Batch 1 Chelator Study

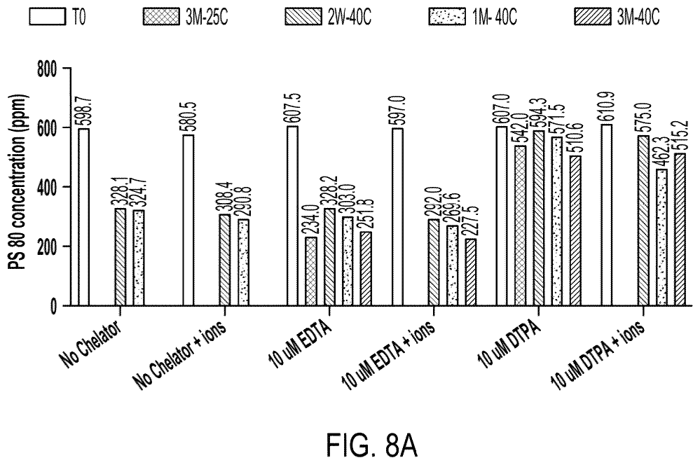

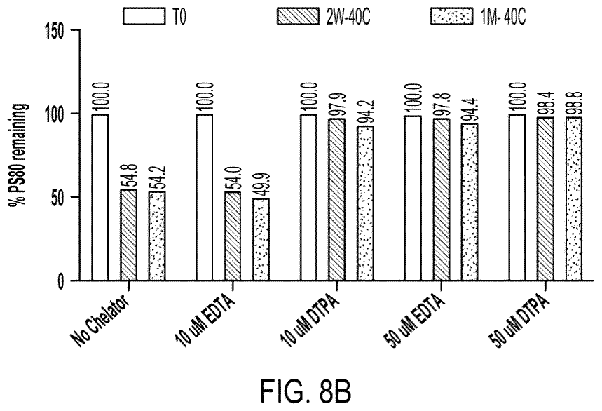

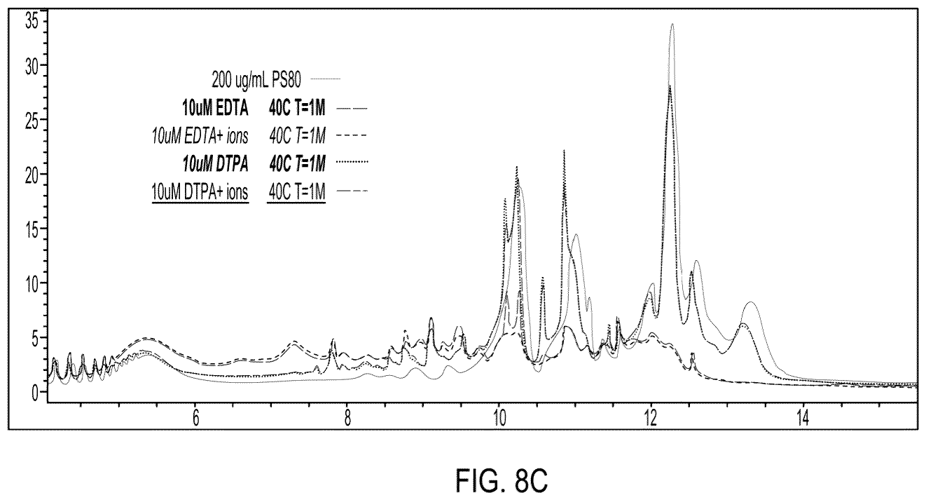

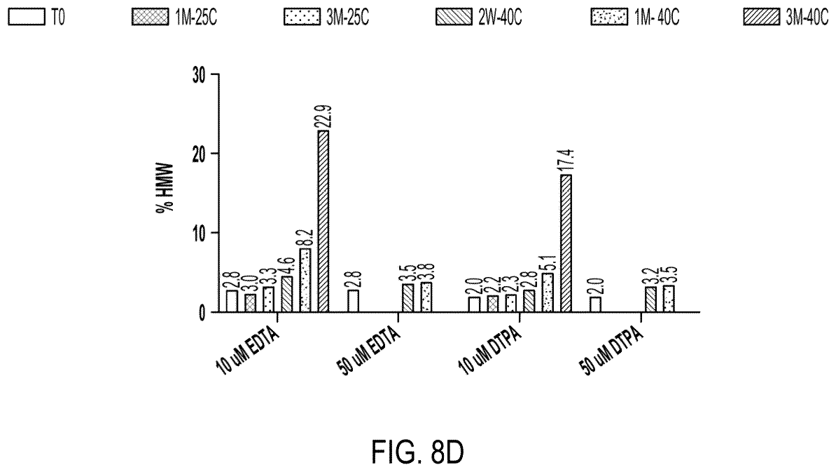

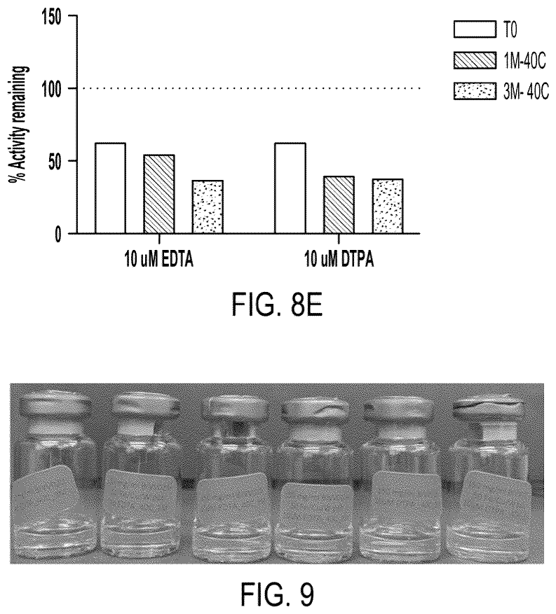

As seen in FIG. 8, PS80 degradation was observed in all samples with 10 μM EDTA with/without metal ions as early as 2 weeks at 40° C., while PS80 degradation was mitigated using 10 μM DTPA even in presence of metal ions for up to 3 months at 40° C. (FIG. 8A). 50 μM EDTA or 50 μM DTPA eliminated PS80 degradation (FIG. 8B). Further, ester profile analysis revealed oxidation to be the root cause of PS80 degradation, and the presence of 10 μM DTPA prevented PS80 oxidation (FIG. 8C). The tendency to form HMW species was greatest at 10 μM EDTA. HMW species formation was lower with 10 μM DTPA than with 10 μM EDTA. 50 μm EDTA and DTPA prevented HMW species formation to a greater extent than 10 μM EDTA, but the difference was less pronounced compared to 10 μM DTPA (FIG. 8D). Solution coloration was also observed at 10 μM EDTA as compared to any other tested group (FIG. 9). Biacore analysis revealed that EDTA and DTPA have no direct impact on the potency of the molecule. A slight decrease in potency was observed over time at 40° C. (FIG. 8E). Chelator had no major effect on the tendency of the molecule to undergo LC D32 isomerization, heavy chain (HC) M251 oxidation, HC PENNYK deamidation or HC VSNK deamidation.

### Batch 2 Chelator Study

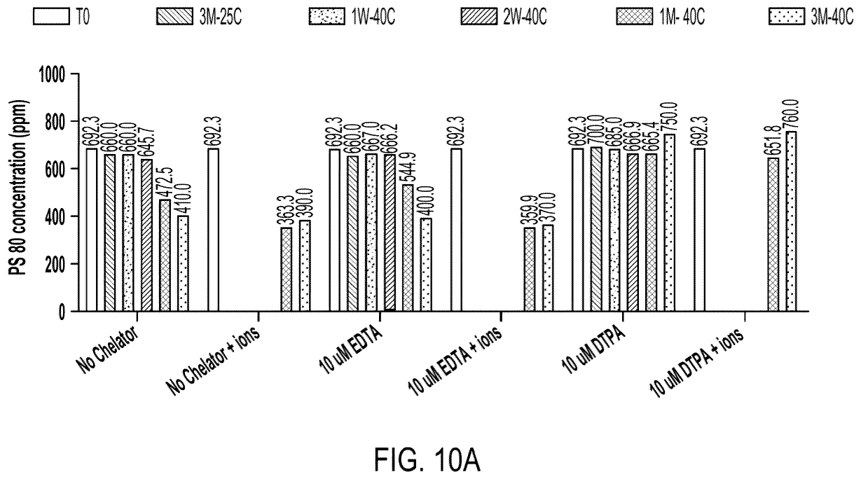

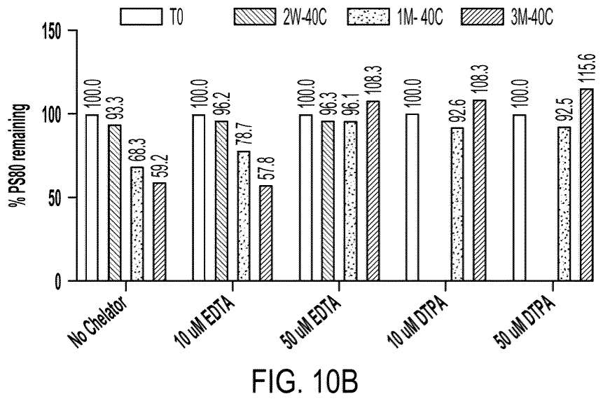

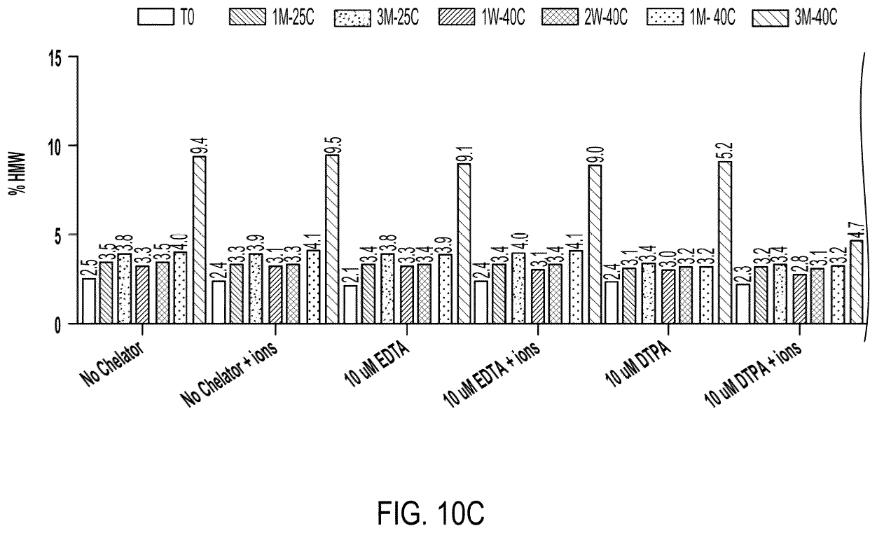

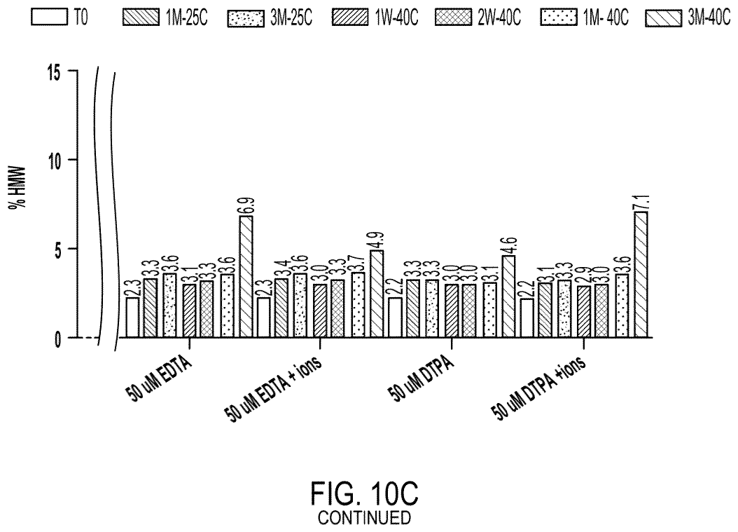

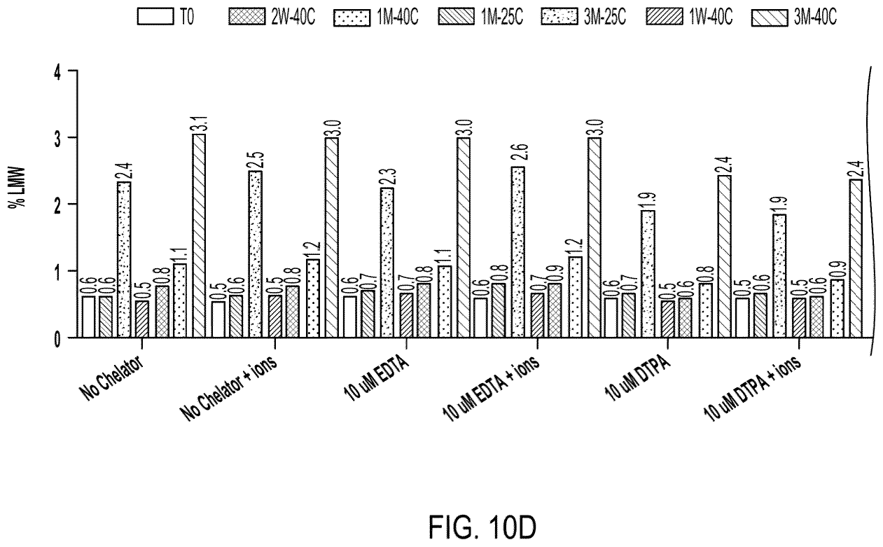

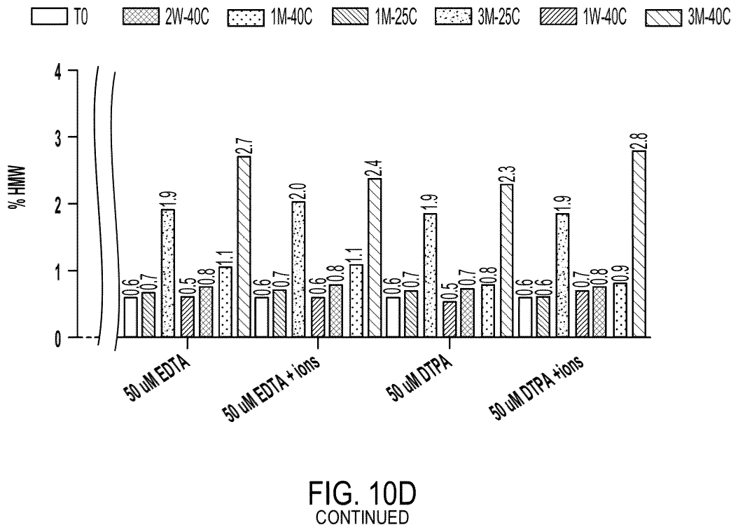

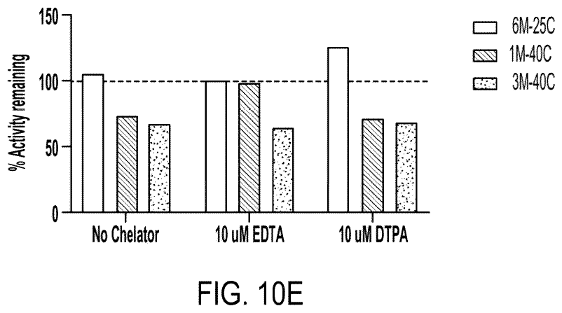

As seen in FIG. 10, PS80 degradation was observed in all samples with 10 μM EDTA with/without ions as early as 2 weeks at 40° C., while PS80 degradation was mitigated using 10 μM DTPA even in presence of metal ions for up to 3 months at 40° C. (FIG. 10A). 50 μM EDTA or 50 μM DTPA eliminated PS80 degradation (FIG. 10B). HMW species formation was greatest at 10 μM EDTA. HMW species formation was less apparent at 10 μM DTPA than at 10 μM EDTA. 50 μm EDTA and DTPA prevented HMW species formation to a greater extent than 10 μM EDTA, but 50 μm EDTA prevented HMW species formation to a similar extent as 10 μM DTPA (FIG. 10C). No significant trends were observed for LMW species formation as a function of chelator (FIG. 10D). Solution coloration was also observed with 10 μM EDTA as compared to any other tested group. Biacore analysis revealed that EDTA and DTPA have no direct impact on the potency of the molecule, as no loss of potency was observed in the presence or absence of the chelators after subjection of samples to 25° C. for 6 months. A slight decrease in potency was observed over time at 40° C. (FIG. 10E). Chelator had no major effect on the tendency of the molecule to undergo LC D32 isomerization, HC M251 oxidation, HC PENNYK deamidation or HC VSNK deamidation.

### Batch 3 Chelator Study

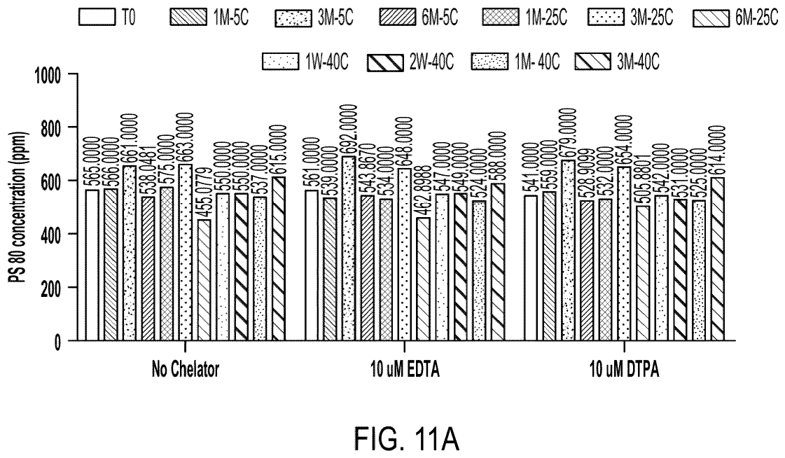

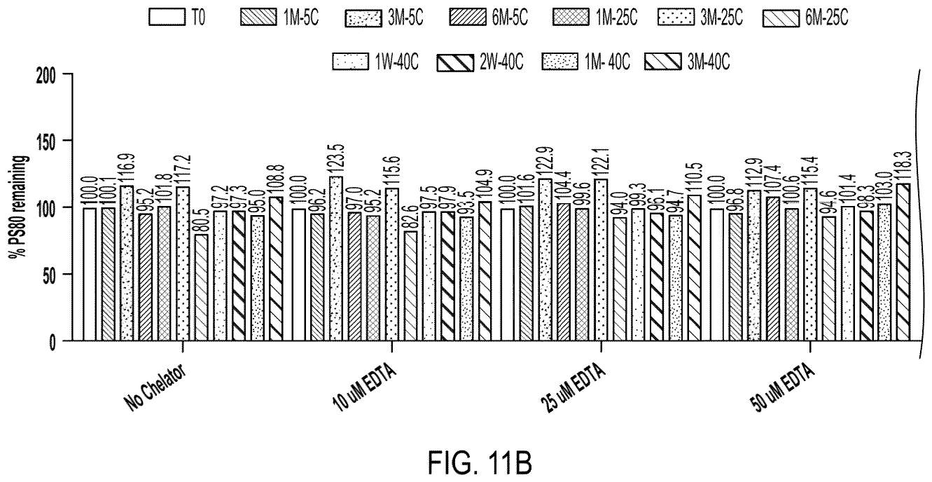

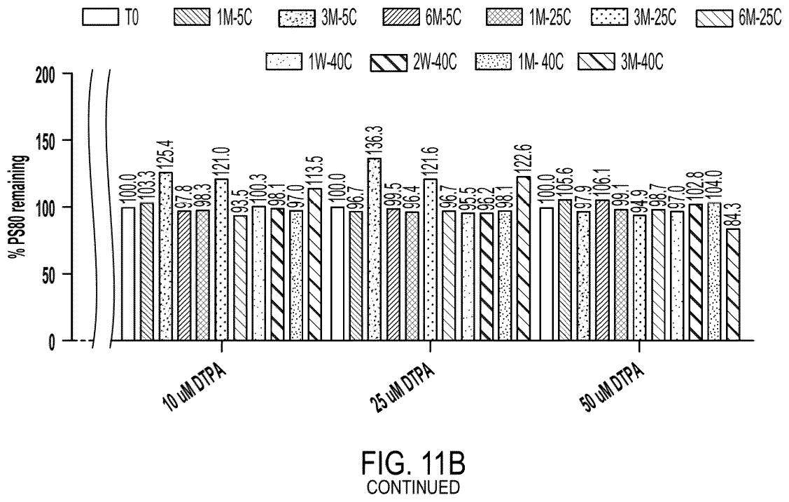

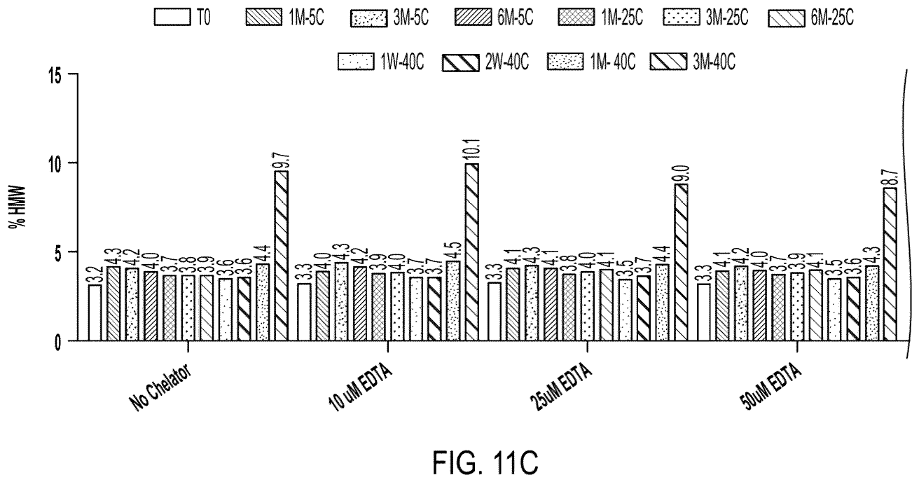

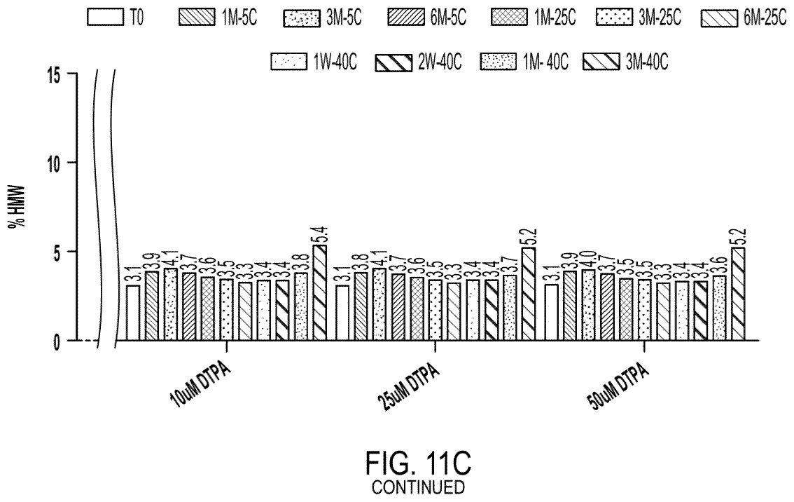

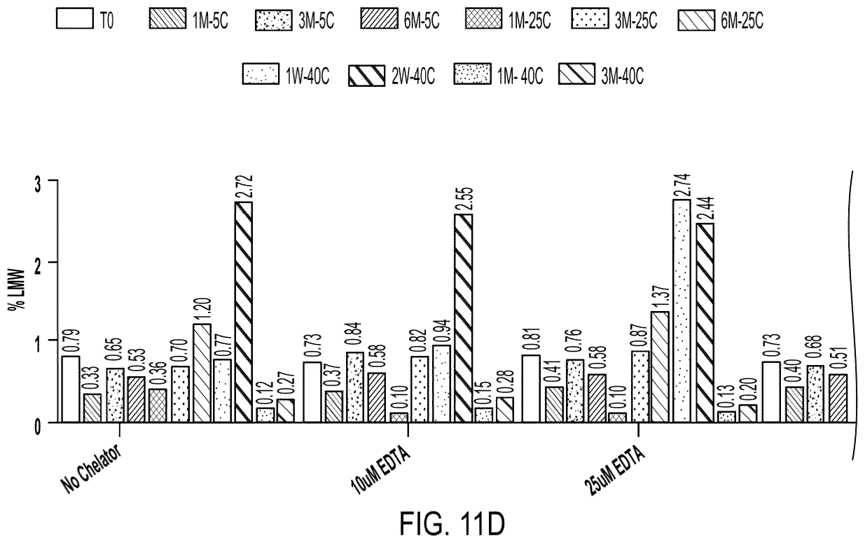

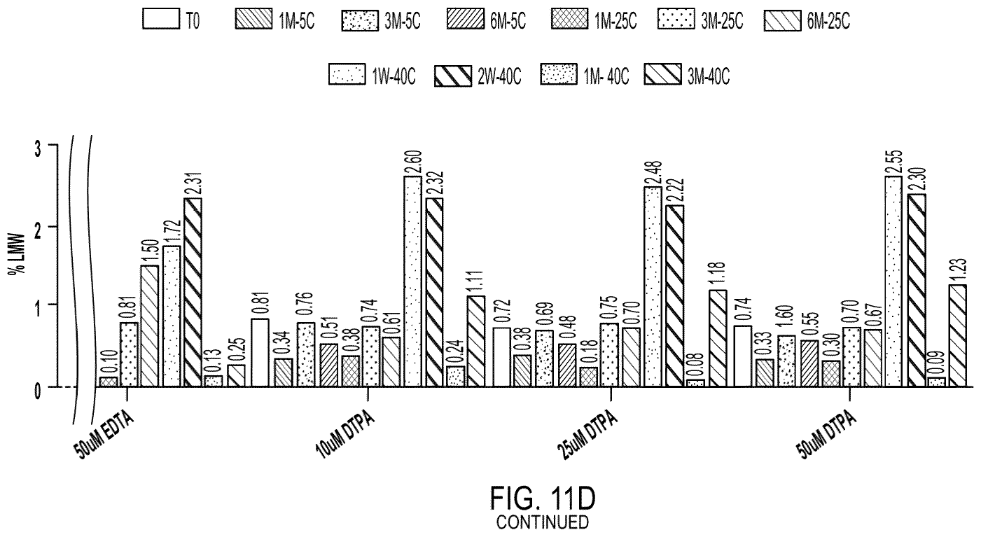

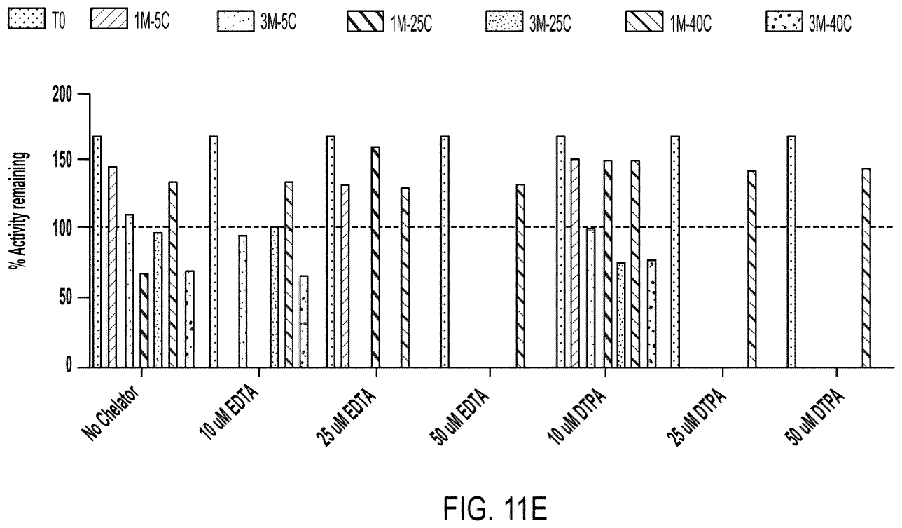

As seen in FIG. 11, less PS80 degradation was observed for all samples compared to the previous batches. Some degree of PS80 degradation was observed for the formulations containing no chelator or 10 μM EDTA compared to 10 μM DTPA after storage for 6 months at 25° C. (FIGS. 11A-B). HMW species formation was greater for formulations containing either no chelator or 10 μM EDTA. Less HMW species formation was apparent at 10 μM DTPA than at 10 μM EDTA (FIG. 11C). No significant trends were observed for LMW species formation as a function of chelator. An overall increase in LMW species formation as a function of storage time and temperature was observed for all groups tested (FIG. 11D). No change in solution coloration was observed for any samples at 5° C. or 25° C.but was observed at 40° C. Biacore analysis revealed that EDTA and DTPA have no direct impact on the potency of the molecule, which retains substantial potency in either chelator after being subjected to 25° C. for 3 months. A slight decrease in potency was observed over time at 40° C. (FIG. 11E). Chelator had no major effect on the tendency of the molecule to undergo LC D32 isomerization, HC M251 oxidation, HC PENNYK deamidation or HC VSNK deamidation.

### Batch 4 Chelator Study

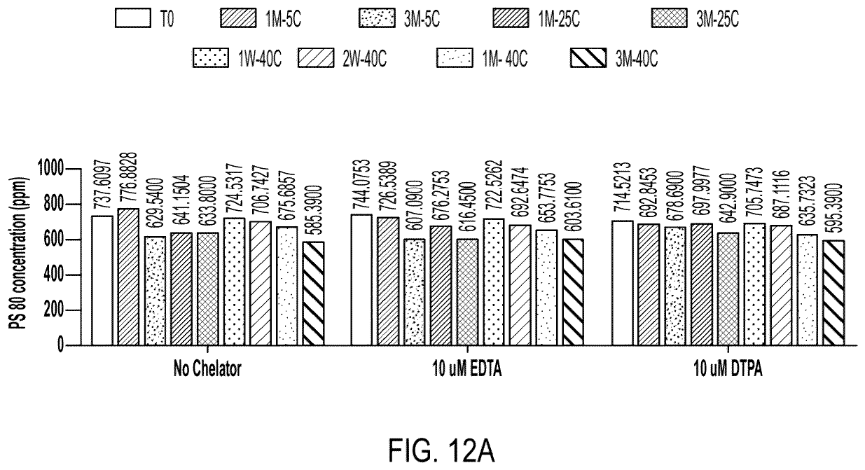

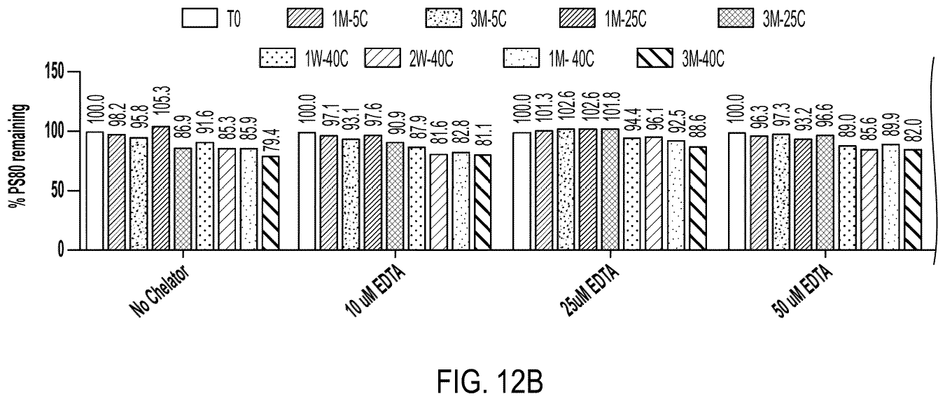

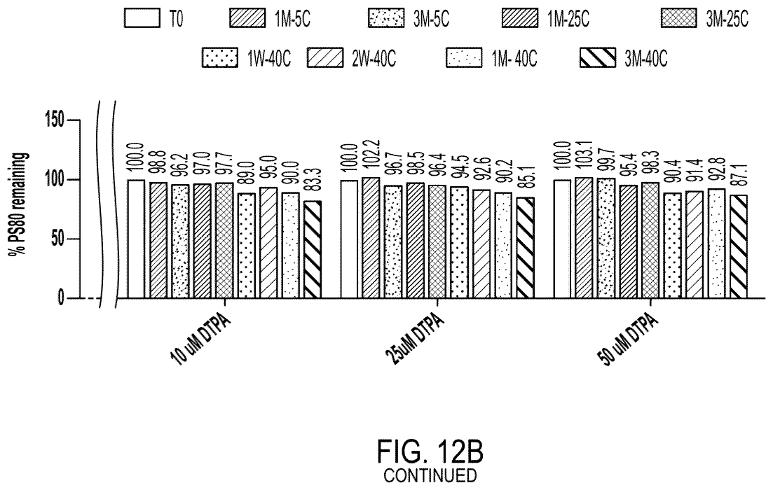

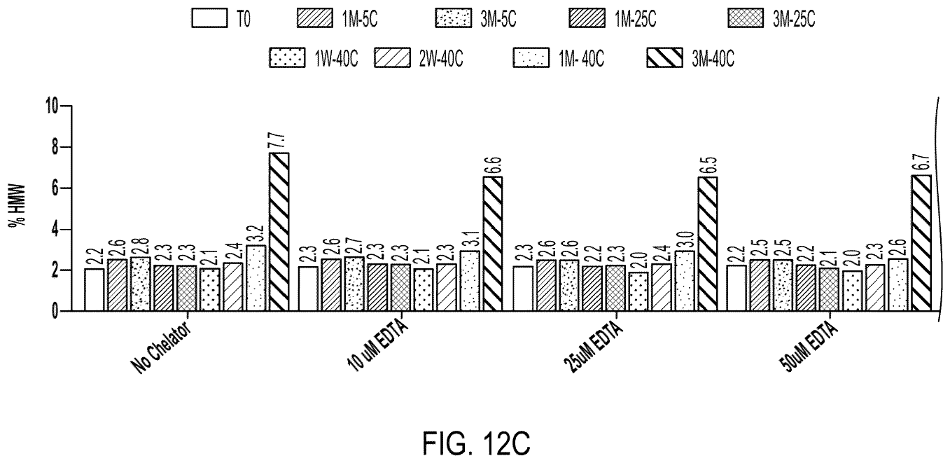

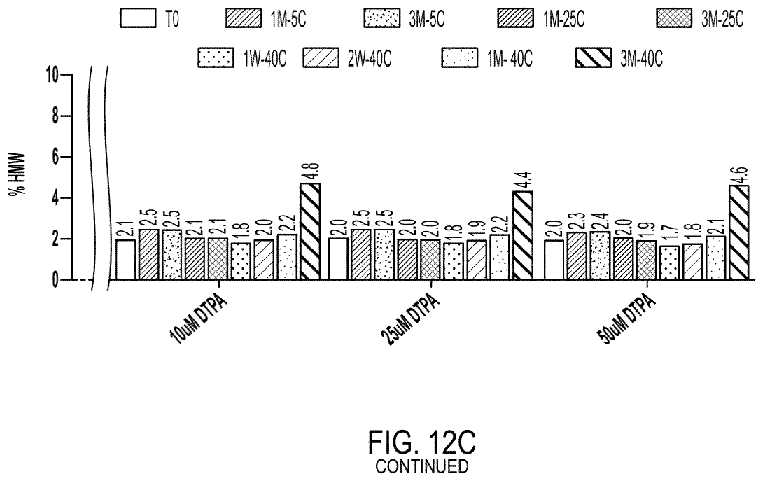

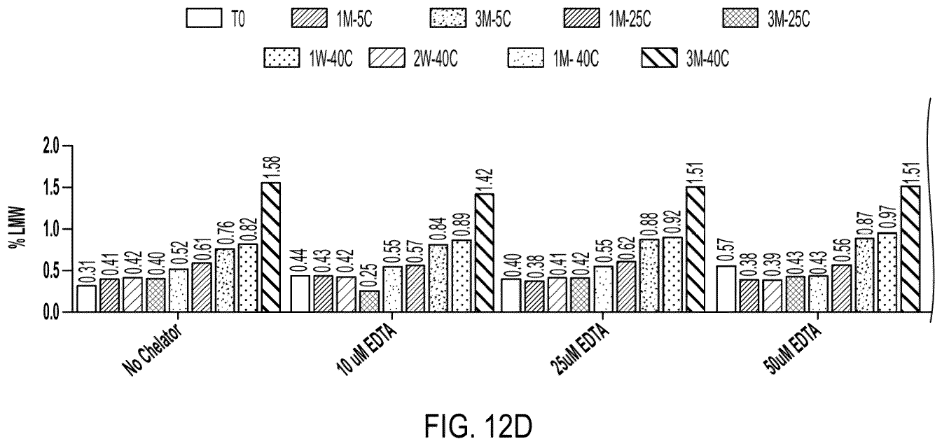

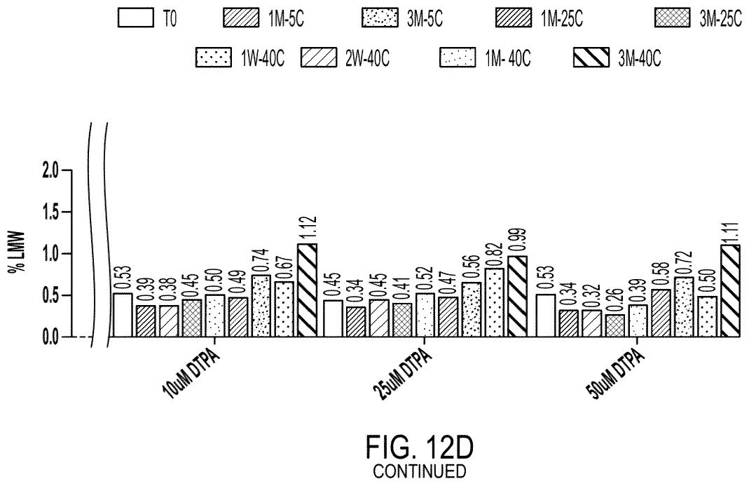

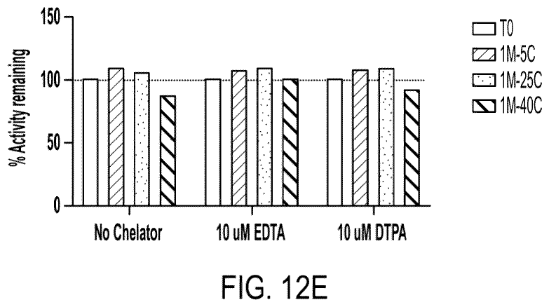

As seen in FIG. 12, less PS80 degradation was observed for all samples compared to the previous batches. All concentrations of EDTA and DTPA appeared to mitigate PS80 degradation to similar extents (FIGS. 12A-B). Similar HMW species formation profiles were observed for EDTA- and DTPA-containing formulations held at 5° C. and 25° C. DTPA-containing formulations showed less HMW species formation at 40° C. compared to formulations containing no chelator or EDTA (FIG. 12C). DTPA containing formulations showed lower LMW species formation compared to formulations containing no chelator or EDTA. An overall increase in LMW species formation with storage time and temperature was observed for all groups tested (FIG. 12D). No change in solution coloration was observed for any samples at 5° C. or 25° C. Solution coloration at 40° C. was not apparent in the presence of DTPA as a chelator. Biacore analysis revealed that EDTA and DTPA have no direct impact on the potency of the molecule, which retains substantial potency after being subjected to 25° C. for 1 month. A slight decrease in potency was observed over time at 40° C. (FIG. 12E). Chelator had no major effect on the tendency of the molecule to undergo LC D32 isomerization, HC M251 oxidation, HC PENNYK deamidation or HC VSNK deamidation.

### Summary

Based on comparative stability testing of SAR445088 formulation samples containing DTPA or EDTA, DTPA at a concentration of 10 μM was determined to optimally mitigate PS80 degradation across different batches, prevent HMW species formation and solution coloration, and have minimal impact on the molecule's ability to maintain potency.

### Example 6. pH Screen/DP Stability Studies

An optimal buffer system and target pH for an SAR445088 formulation were identified by subjecting different batches of SAR445088 to refrigerated, ambient, accelerated and frozen-storage stability testing. This testing was conducted using different primary containers, i.e., the vials and the pre-filled syringes (PFS) as described in Table 4, to test stability of SAR445088 liquid DP in these containers in parallel.

TABLE 4
pH Screen/DP stability study plan

| FDS | Buffers | Target pH | DP | Temperature | |
|---|---|---|---|---|---|
| Batch | screened | screened | presentations | (° C.) | Timepoints |
| 1 | 10 mM Histidine, | 6.7, 6.8, 7.0, 7.2 | Vials, PFS-BD | −80, −30, | 2 wk, 1 M, 2 M, |
| | 10 mM Phosphate | | Neopak High, | 5, 25, 40 | 3 M, 4.5 M, 6 M, |
| | | | BD Neopak Low, | | 12 M |
| | | | OMPI | | |
| 2 | 10 mM Histidine | 6.4, 6.8, 7.1 | Vials, PFS-OMPI | −80, −30, | 2 wk, 1 M, 2 M, |
| | | | | 5, 25, 40 | 3 M, 6 M, 12 M |
| 3 | 10 mM Histidine | 6.4, 6.8, 7.1 | Vials, PFS-OMPI | 5, 25, 40 | 2 wk, 1 M, 2 M, |
| | | | | | 3 M, 6 M |
| 4 | 10 mM Histidine | 6.4, 6.8, 7.1 | Vials | 5, 25, 40 | 2 wk, 1 M, 2 M, |
| | | | | | 3 M |
| 5 | 10 mM Histidine | 6.8 | Vials | 5, 25, 40 | 2 wk, 1 M |

### Batch 1 Formulation Study

### PFS Stability Study

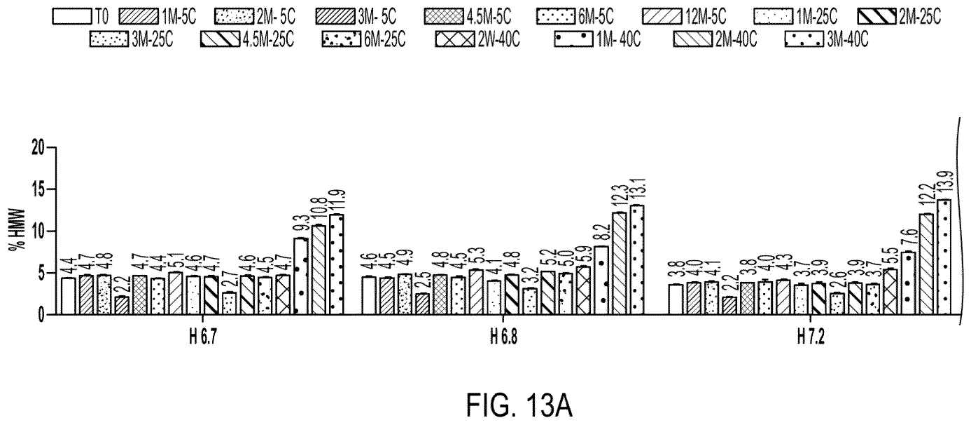

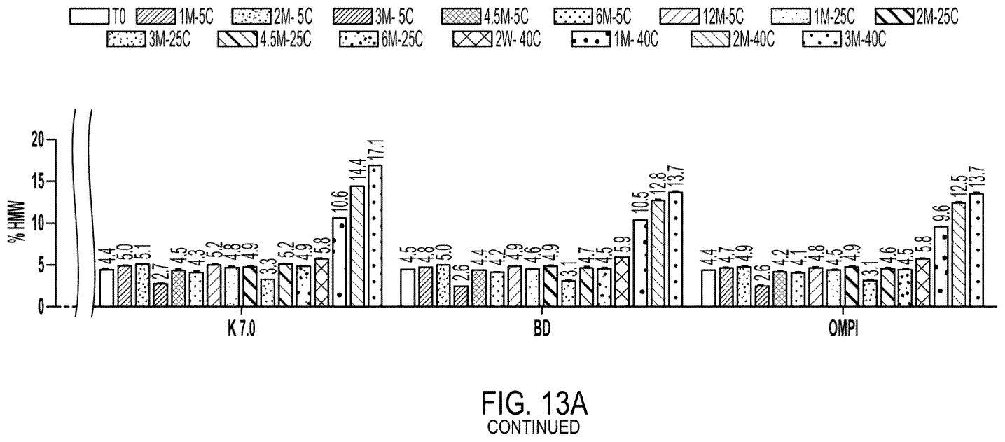

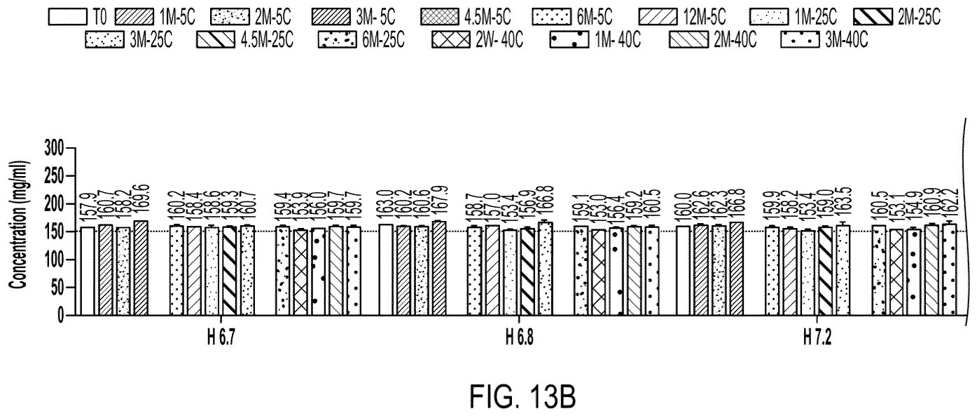

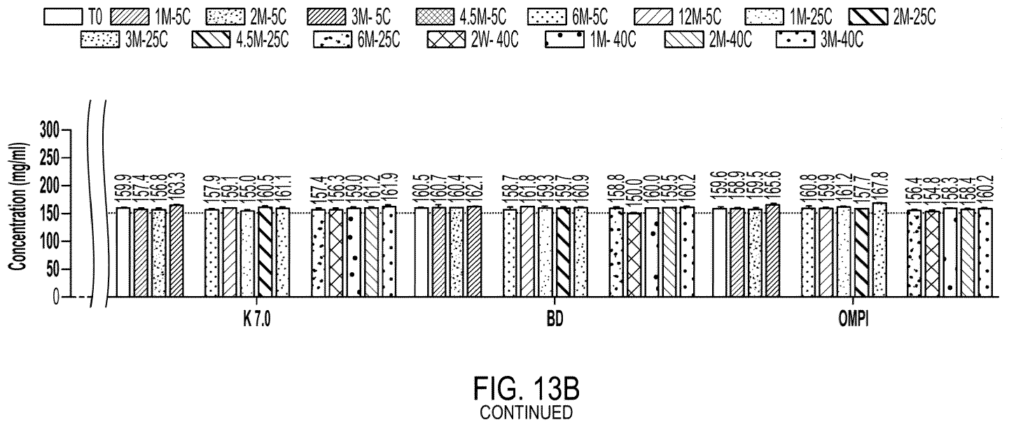

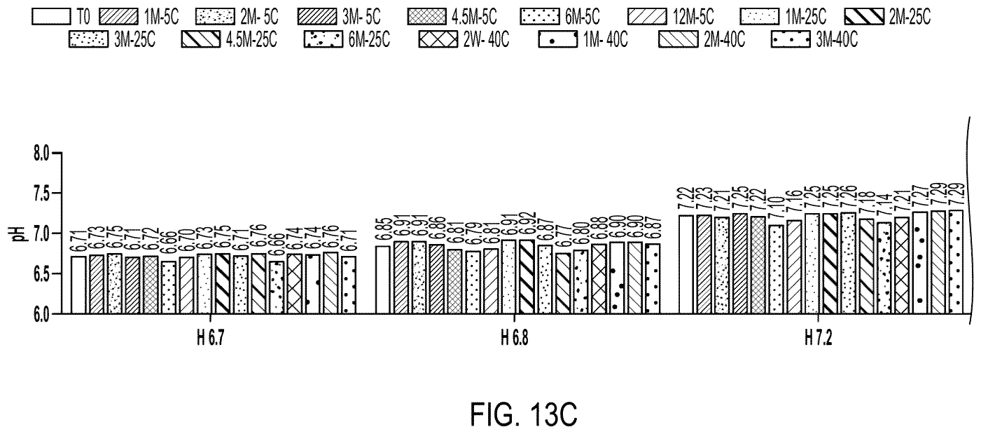

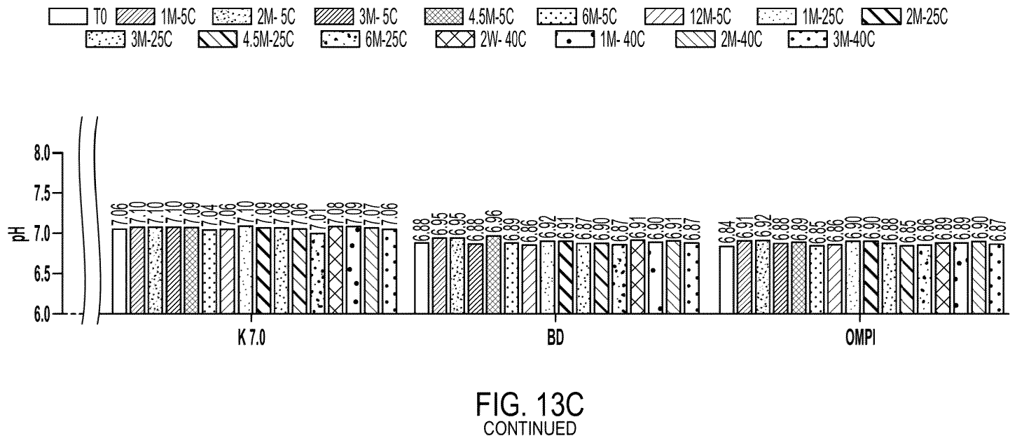

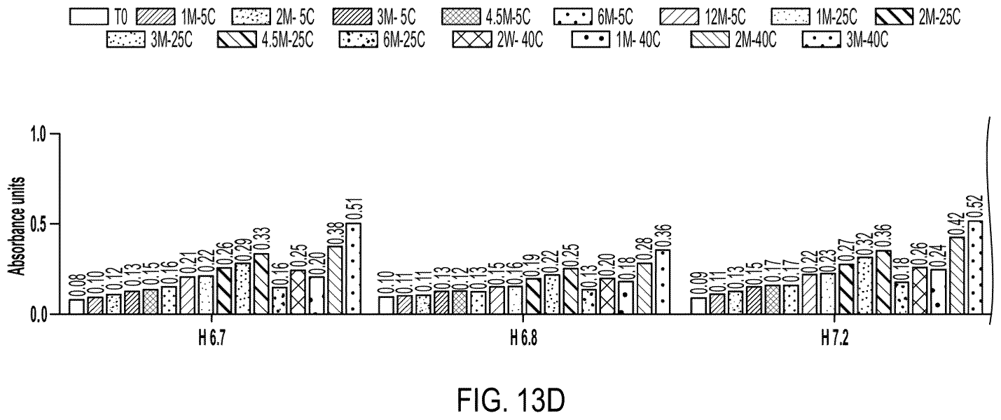

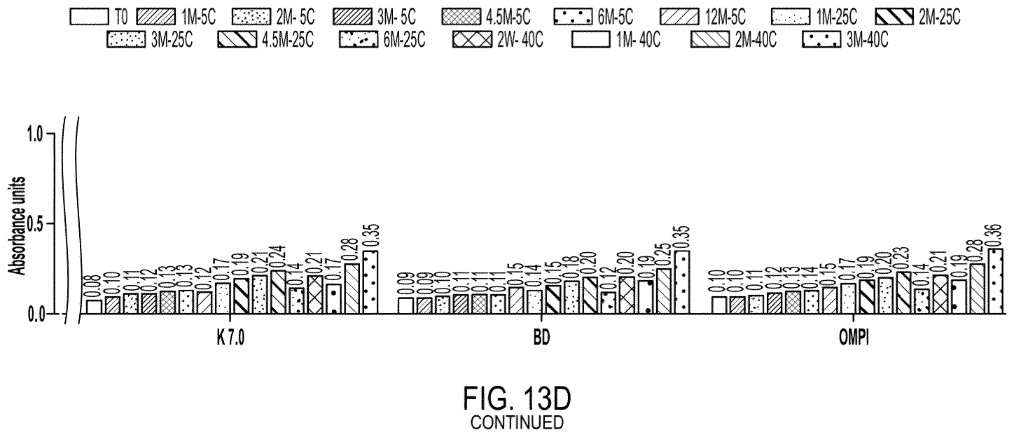

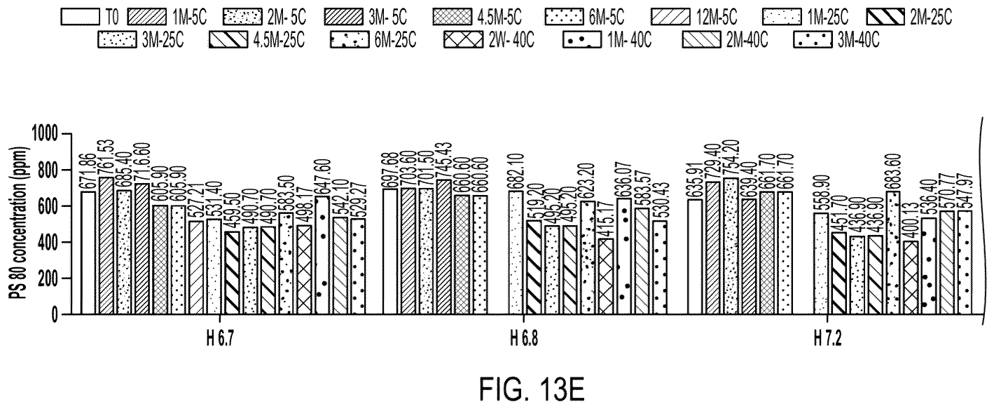

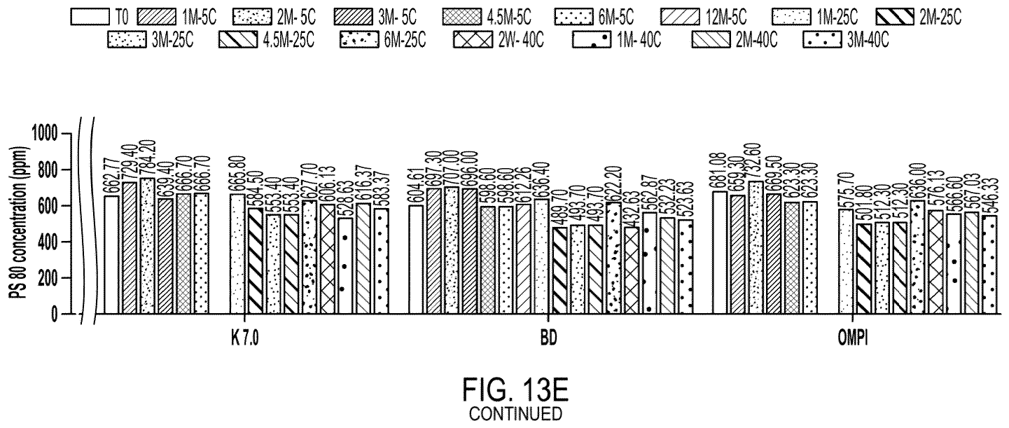

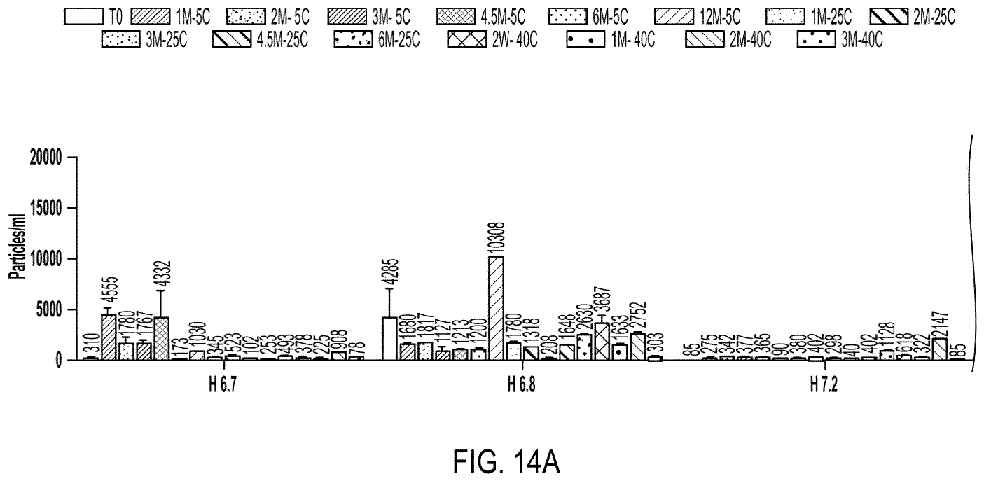

As shown in FIG. 13, SAR445088 in potassium phosphate buffer had a higher propensity to form HMW species than in histidine buffer under accelerated conditions at 40° C. Overall, at all pH and buffer conditions tested, SAR445088 maintained substantial stability during ambient (25° C.) and refrigerated storage (5° C.). No significant difference in HMW species formation was observed between storage in BD neopak low, BD neopak high and OMPI syringes. A slight decrease in HMW species formation at T0 and subsequent timepoints was observed for histidine buffer at pH 7.2 compared to histidine buffer at 6.7 or 6.8 (FIG. 13A). None of buffer, pH or PFS had an apparent effect on antibody concentration (FIG. 13B) or pH measured at end of stability testing period (FIG. 13C). A general increase in turbidity as a function of time and temperature was observed for all buffers and target pHs. However, no significant differences in turbidity were observed between different pH, buffers or type of syringe tested (FIG. 13D). All formulations showed some degree of PS80 degradation over time for 25° C. and 40° C. storage conditions. Histidine-containing formulations at all pH values showed more PS80 degradation than potassium phosphate-containing formulations (FIG. 13E).

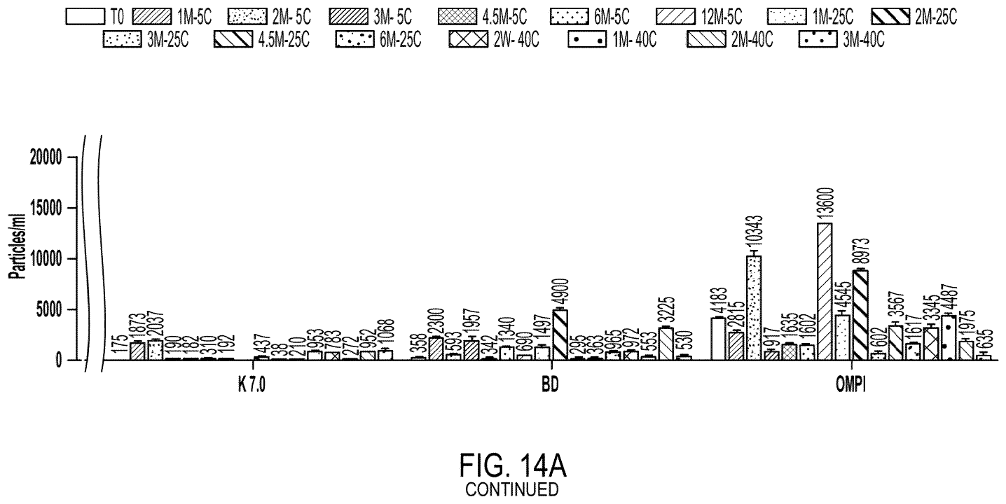

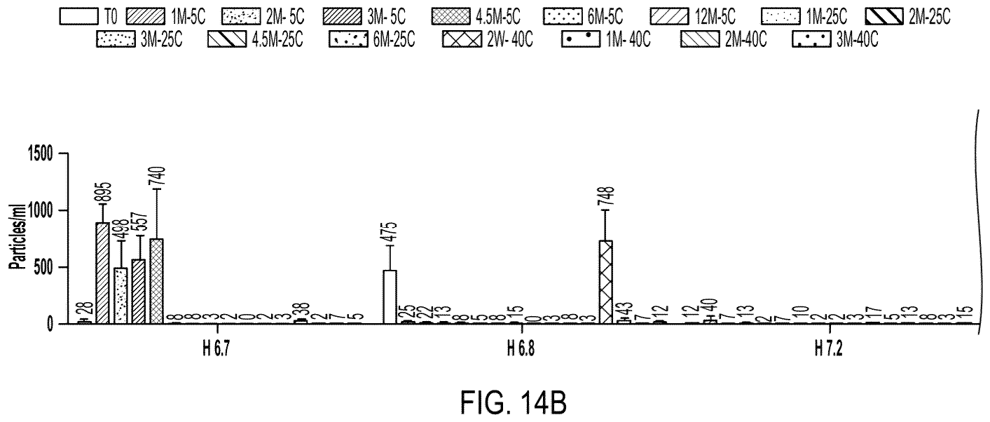

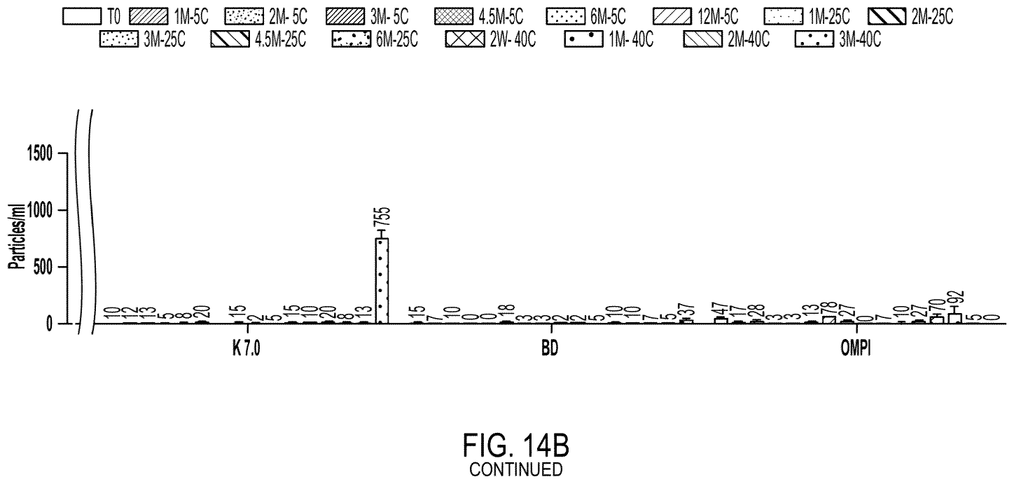

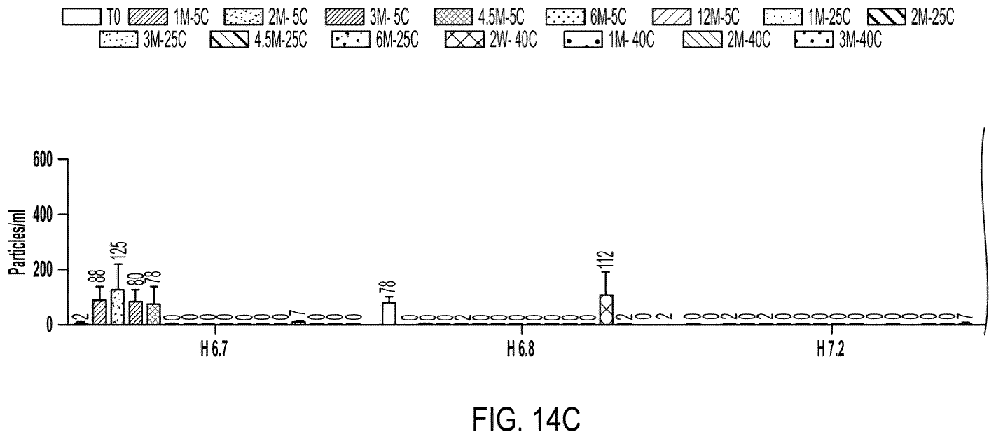

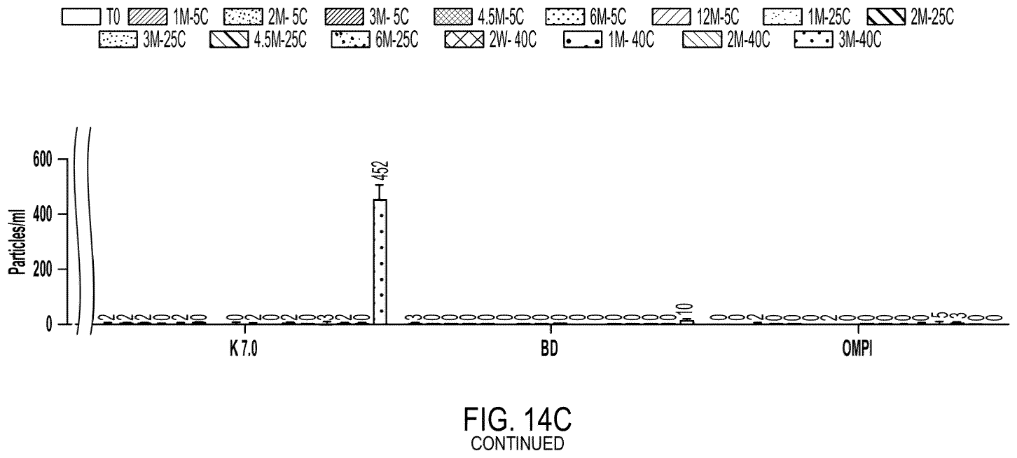

As shown in FIGS. 14, at all tested pH values and buffers, no notable increase in sub-visible particle formation occurred upon storage at different temperatures, with the exception of the potassium phosphate formulation at pH 7.0 (K 7.0) at 3 months at 40° C. 2 μm particle formation was greater in the OMPI PFS compared to its BD counterparts (FIG. 14A). Formation of sub-visible particles≤10 μm and ≤25 μm in size was largely consistent, with a couple of outliers attributed to assay variability (FIGS. 14B-C).

### Vial Stability Study

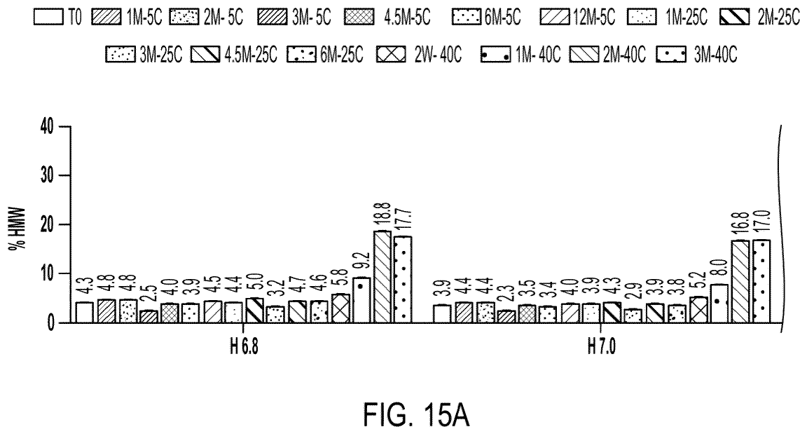

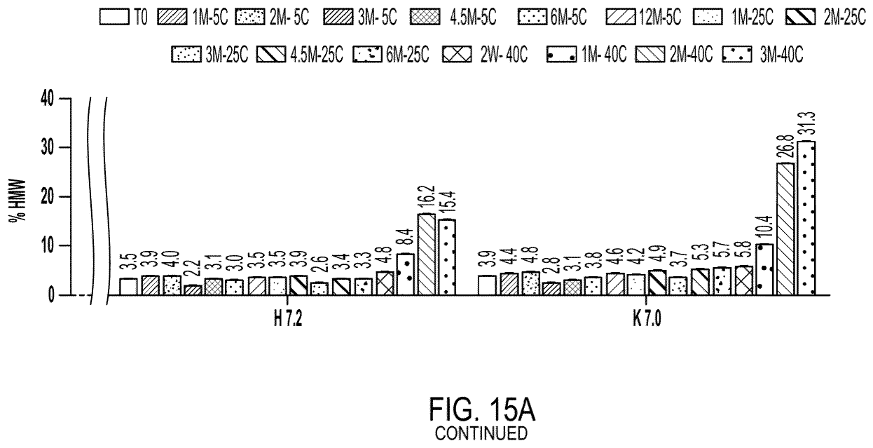

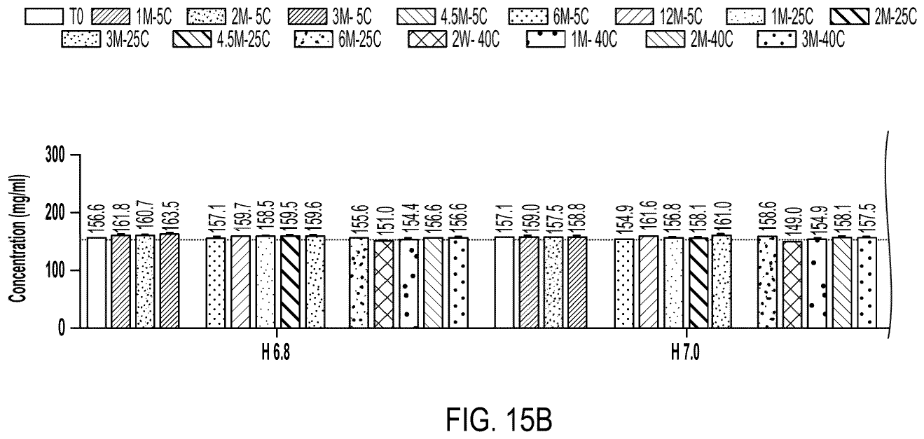

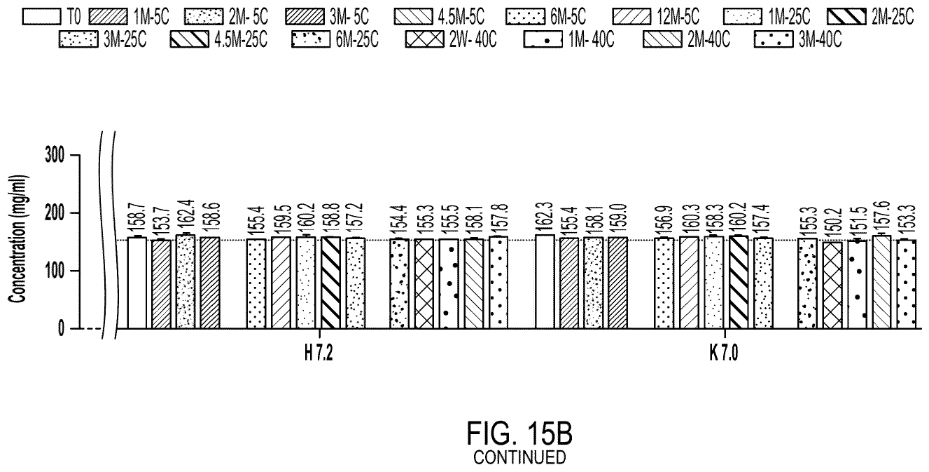

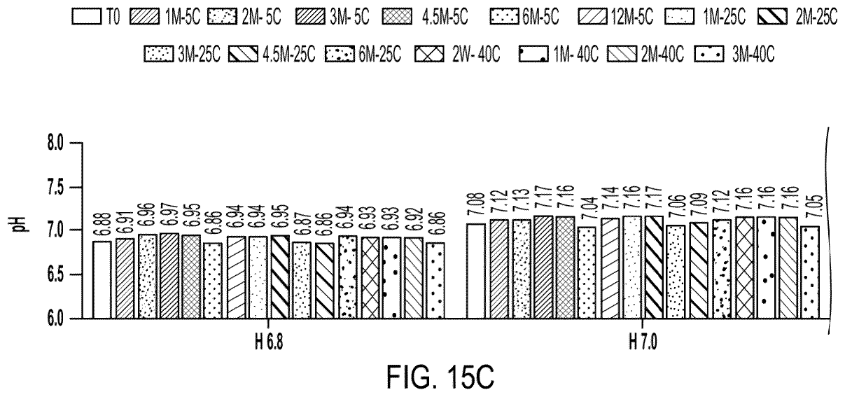

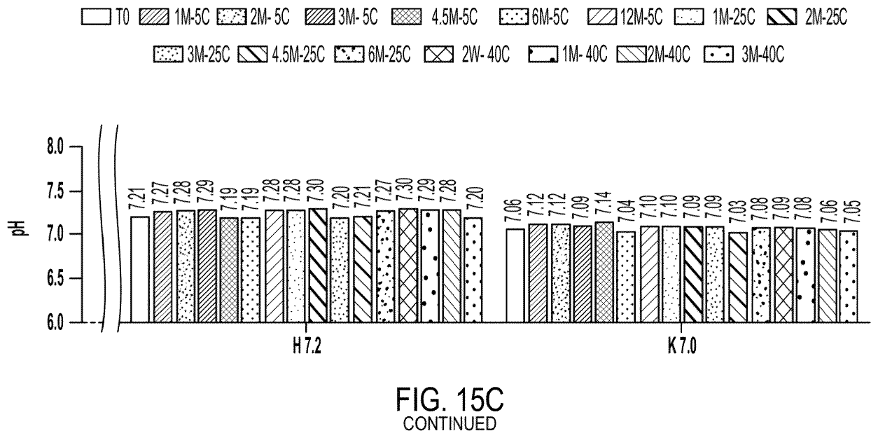

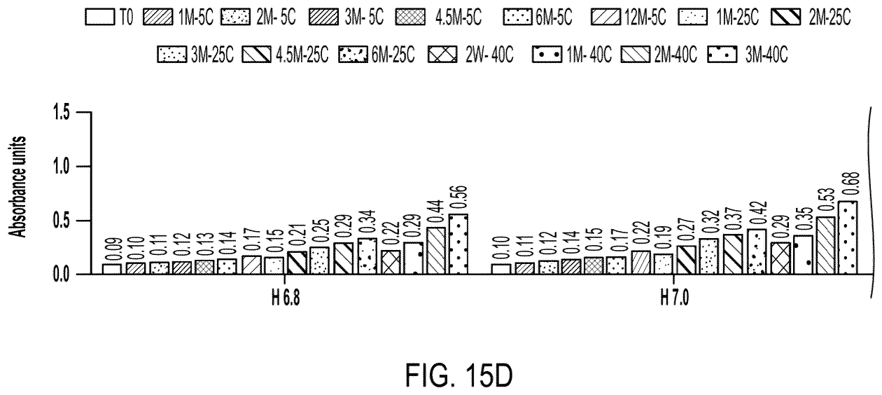

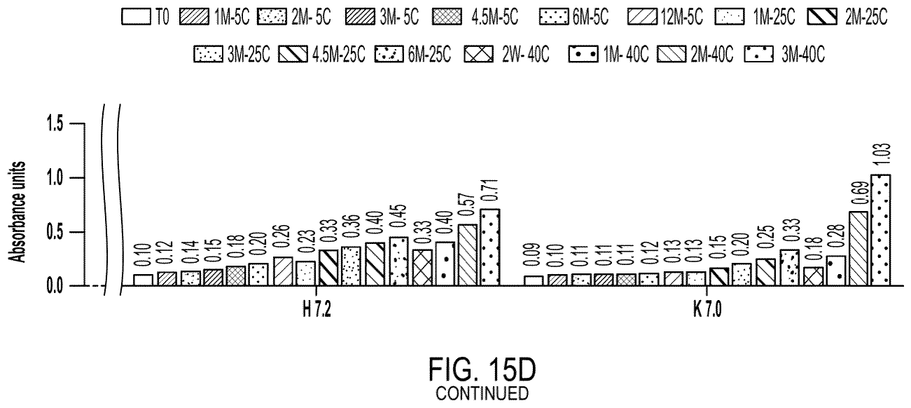

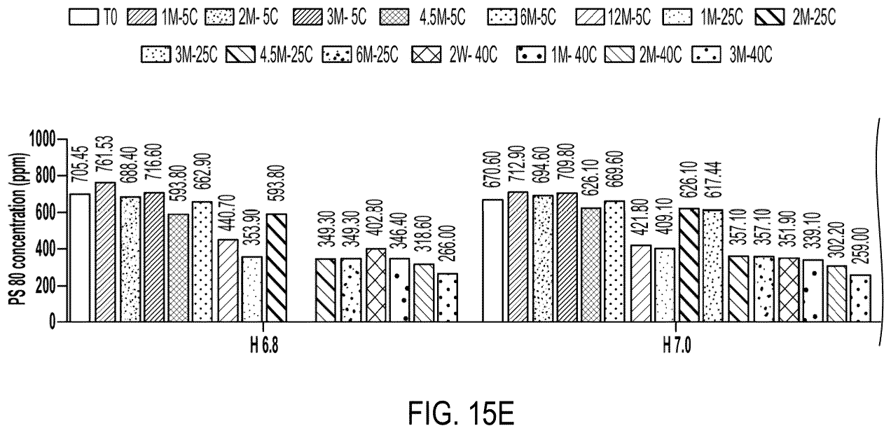

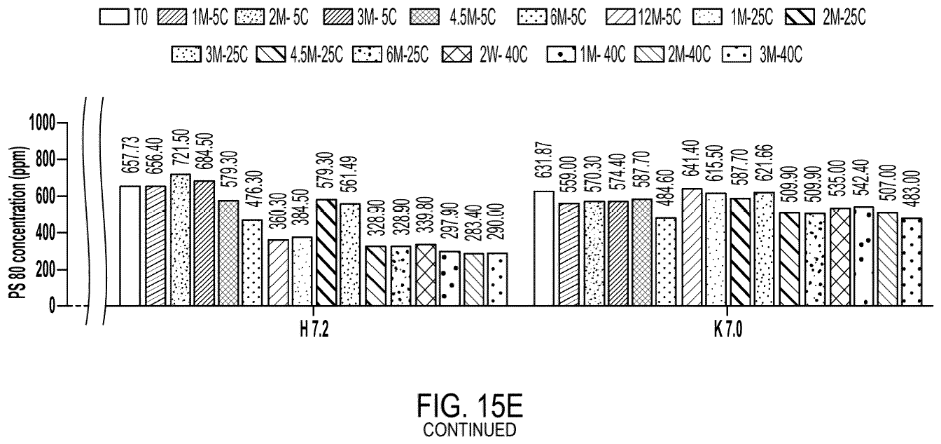

As shown in FIG. 15, SAR445088 had a higher propensity to form HMW species in potassium phosphate buffer than in histidine buffer under accelerated conditions at 40° C. Overall, at all pH and buffer conditions, SAR445088 retained substantial stability upon ambient (25° C.) and refrigerated storage (5° C.). A slight decrease in HMW species formation at T0 and subsequent timepoints was observed with H7.2 buffer compared to H6.7 and H6.8 (FIG. 15A). Neither buffer nor pH had an apparent effect on antibody concentration (FIG. 15B) or pH measured at the end of the testing period (FIG. 15C). A general increase in turbidity as a function of time and temperature was observed for all buffers and target pH values. However, a greater increase in turbidity was apparent in potassium phosphate buffer-containing formulations than in the histidine-containing formulations (FIG. 15D). PS80 degradation was apparent in all formulations over time at 25° C. and 40° C. storage conditions. Histidine-containing formulations at all pHs showed more PS80 degradation than did potassium phosphate-containing formulations (FIG. 15E). PS80 degradation was greater when the same formulation was placed in vials than in a PFS, possibly due to an increased headspace in the vials.

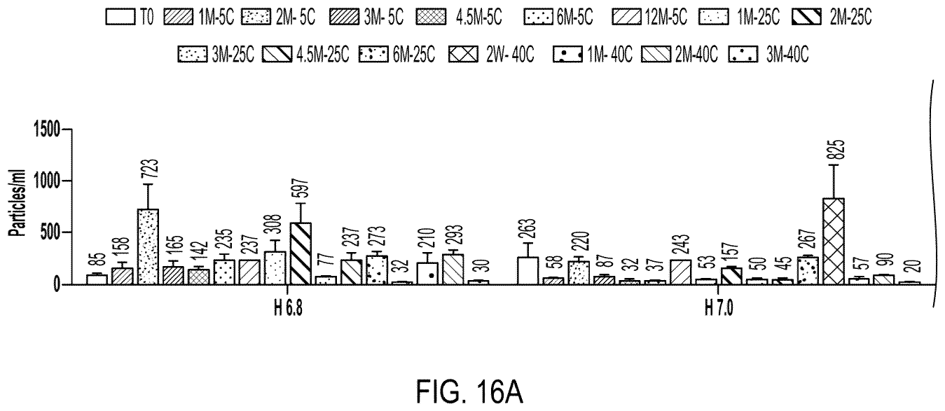

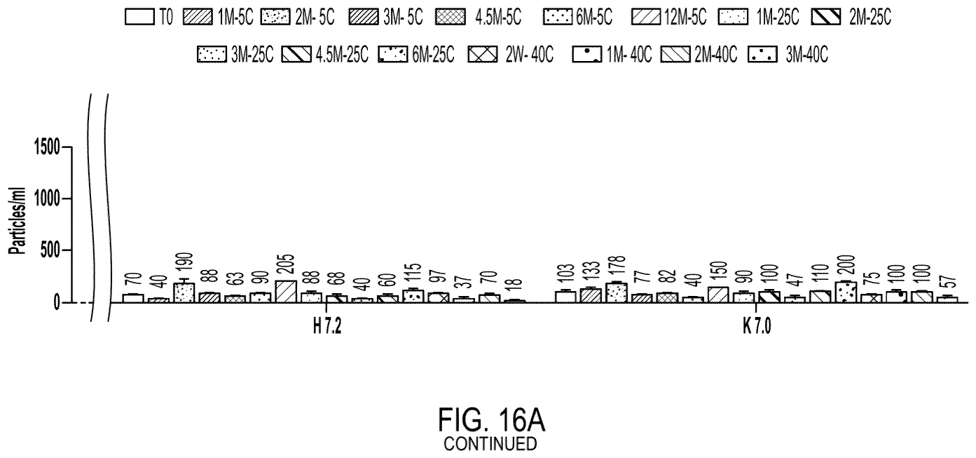

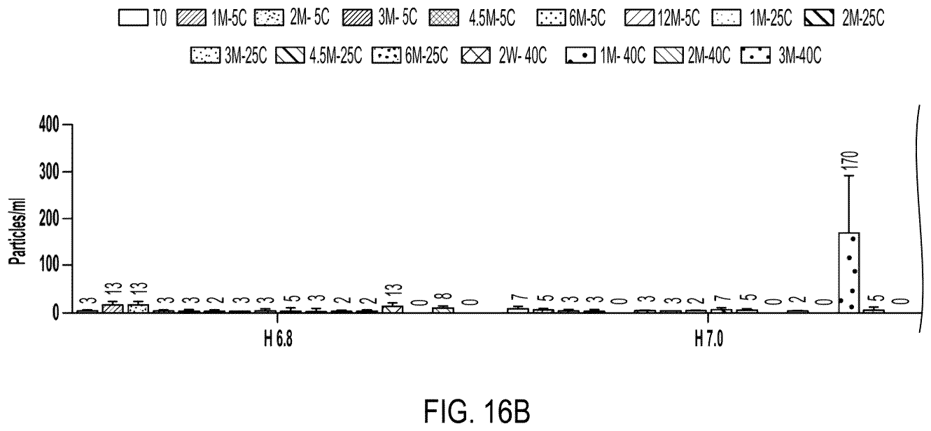

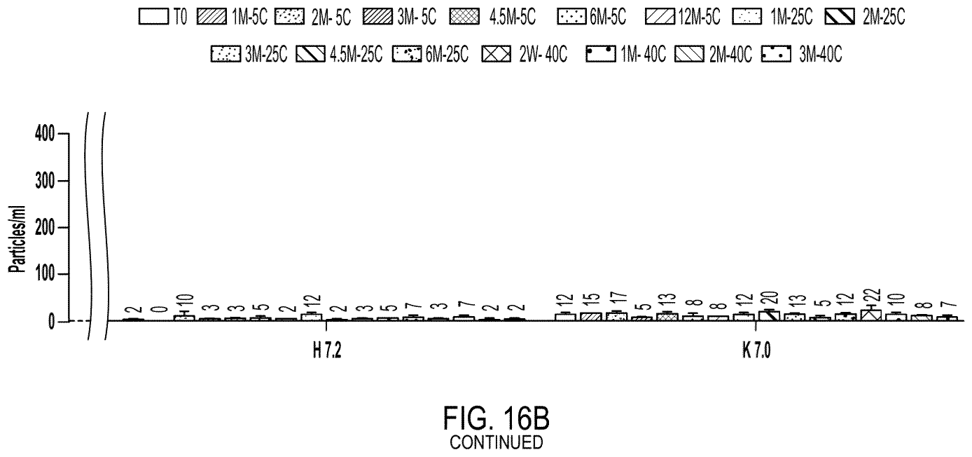

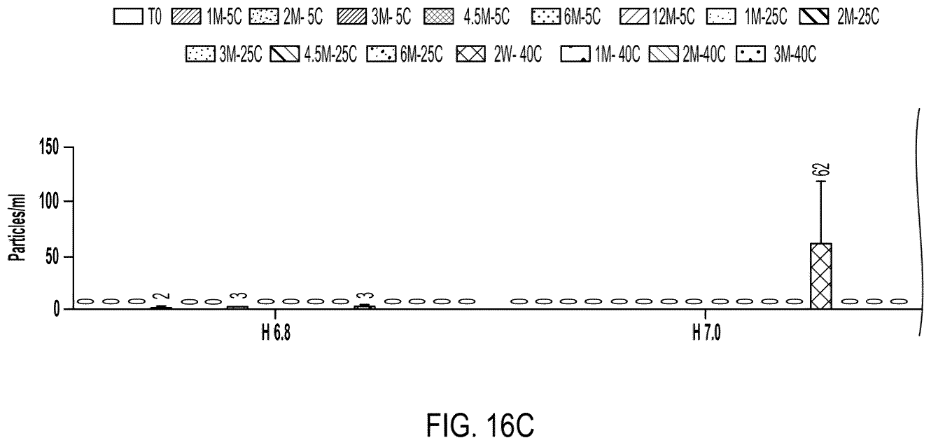

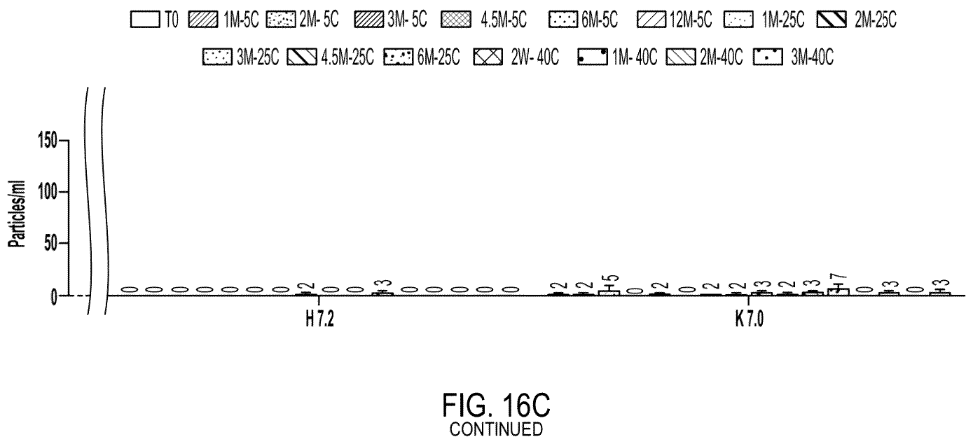

As shown in FIG. 16, for all the tested pH values and buffers, no notable increase in sub-visible particle formation was observed upon storage at different temperatures. Formation of ≤2 μm, ≤10 μm and <25 μm sub-visible particles was largely consistent, with a couple of outliers attributed to assay variability (FIGS. 16A-C).

### Frozen-Storage Stability Study

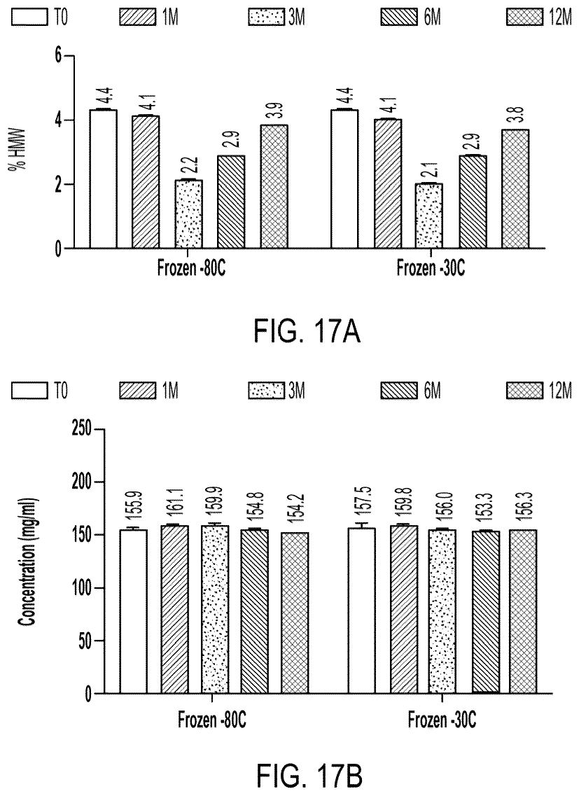

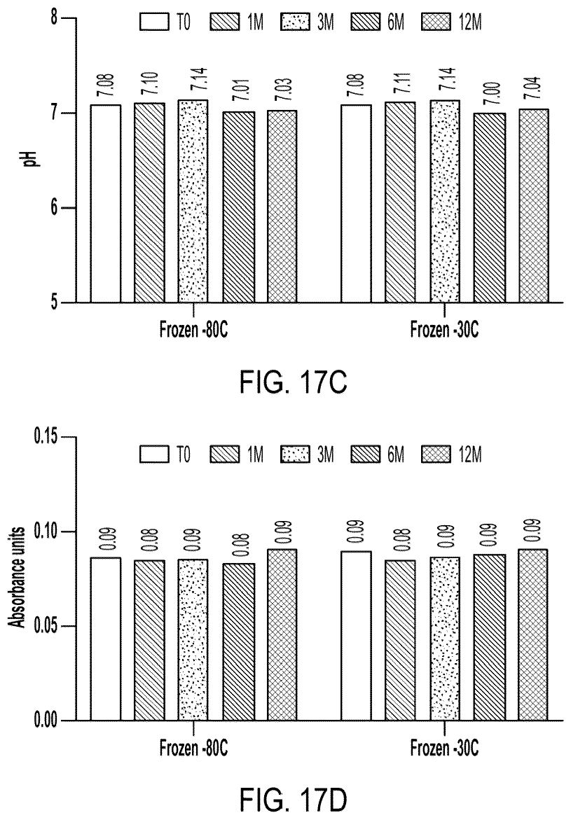

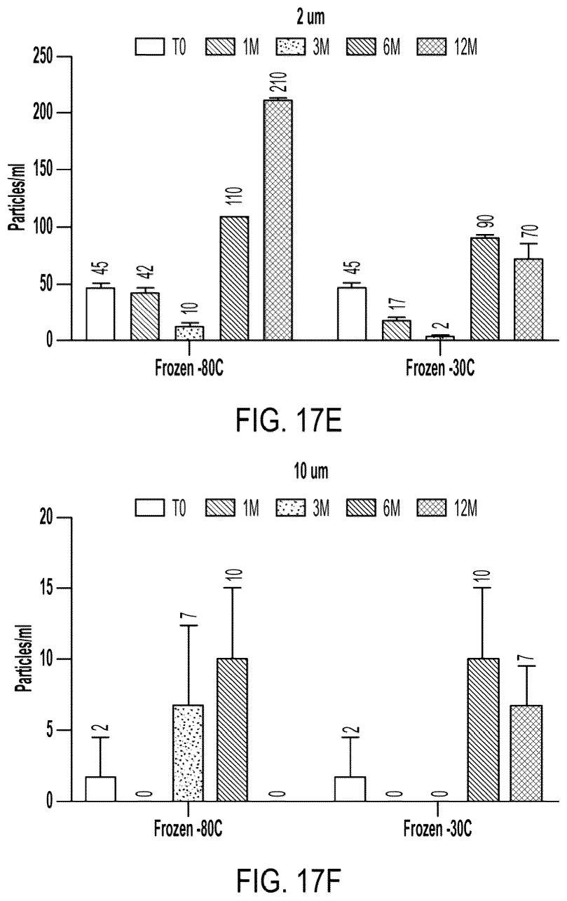

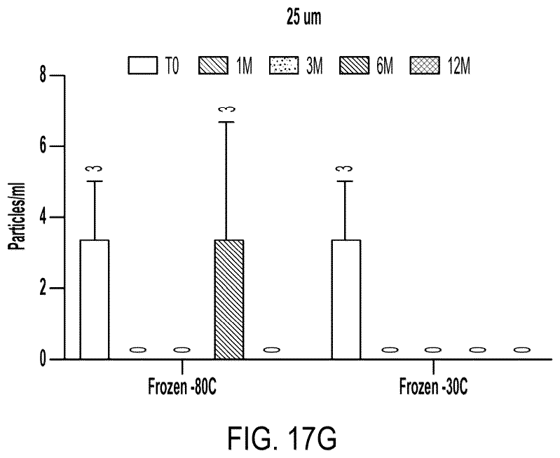

As shown in FIG. 17, a slight decrease in HMW species formation upon frozen storage at −80° C. and −30° C. was observed (FIG. 17A). Frozen storage had no observable effect on antibody concentration (FIG. 17B), pH (FIG. 17C), turbidity (FIG. 17D) and sub-visible particles (FIG. 17E) measured at the end of the storage period. SAR445088 was stable up to 12 months in frozen storage.

### Peptide Map and Biacore Binding Analysis

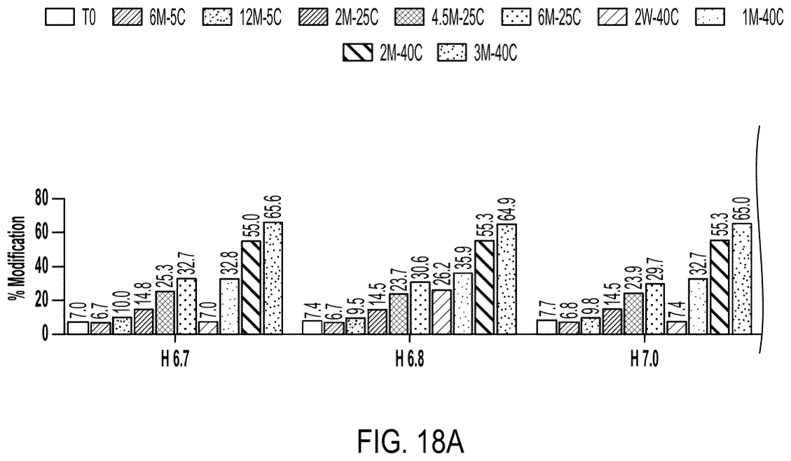

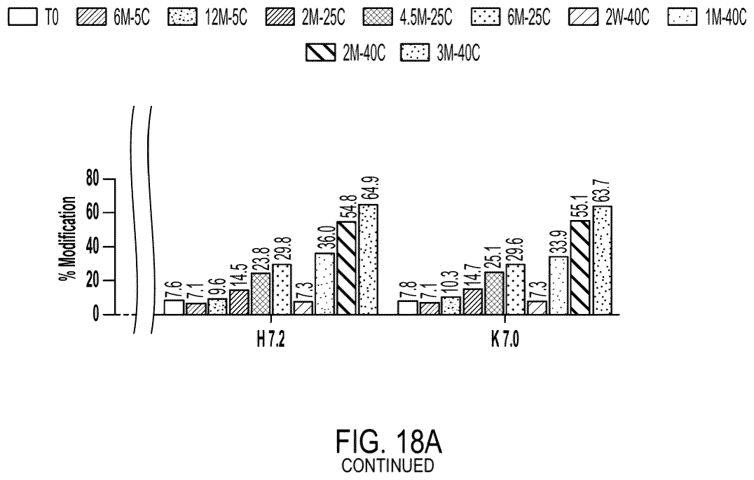

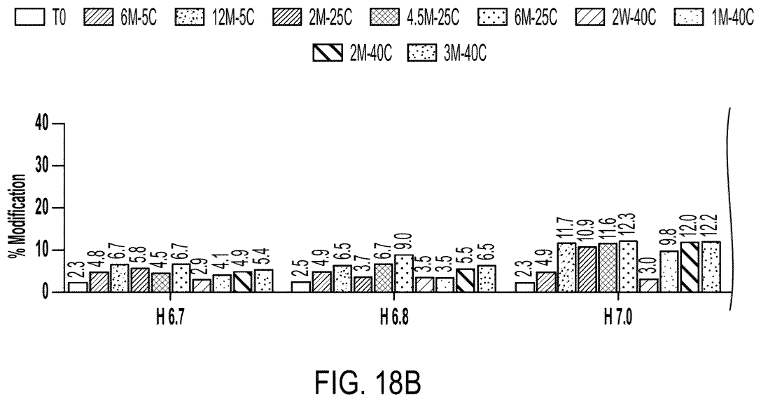

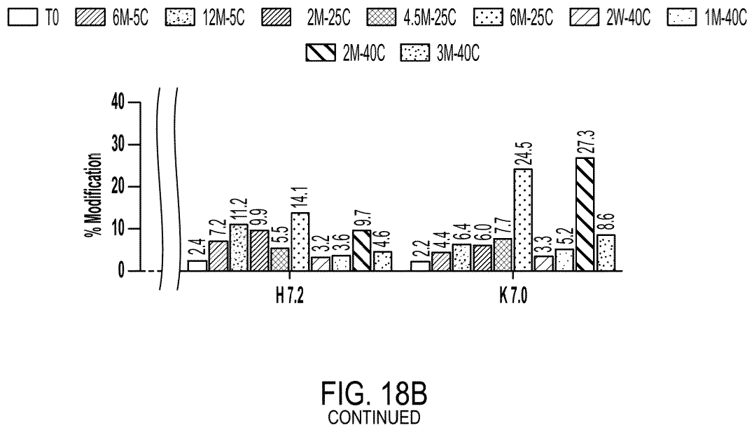

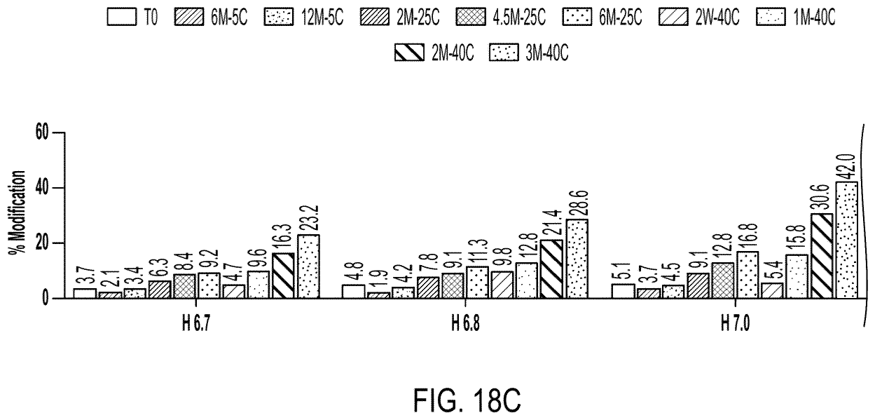

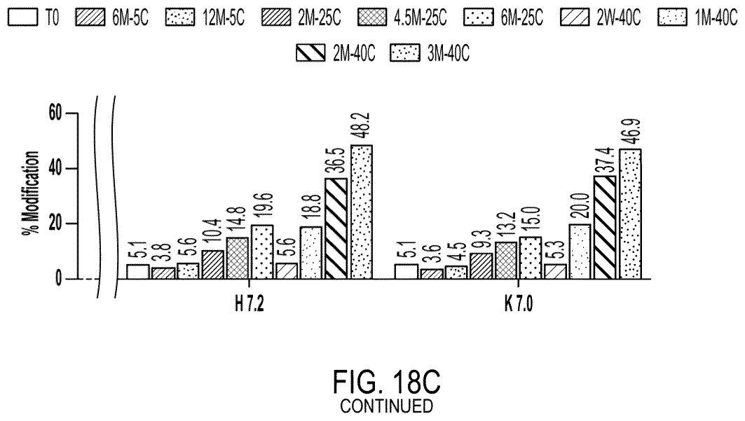

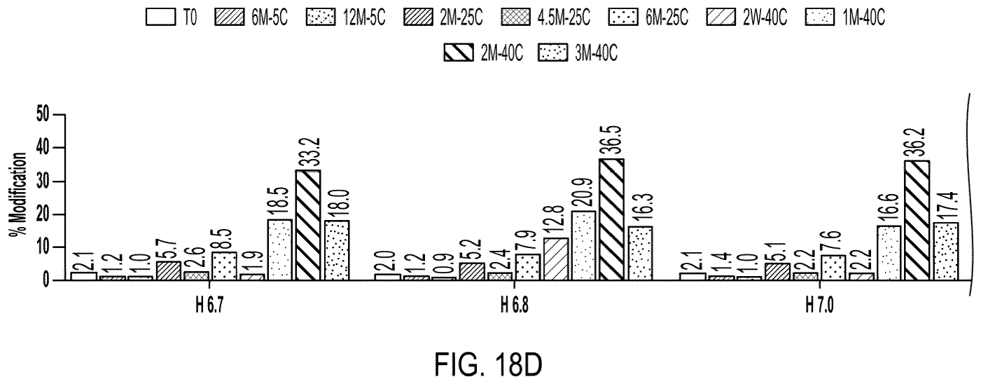

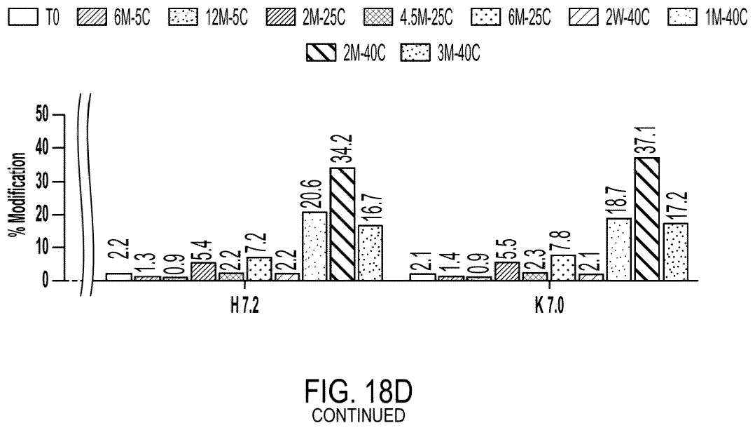

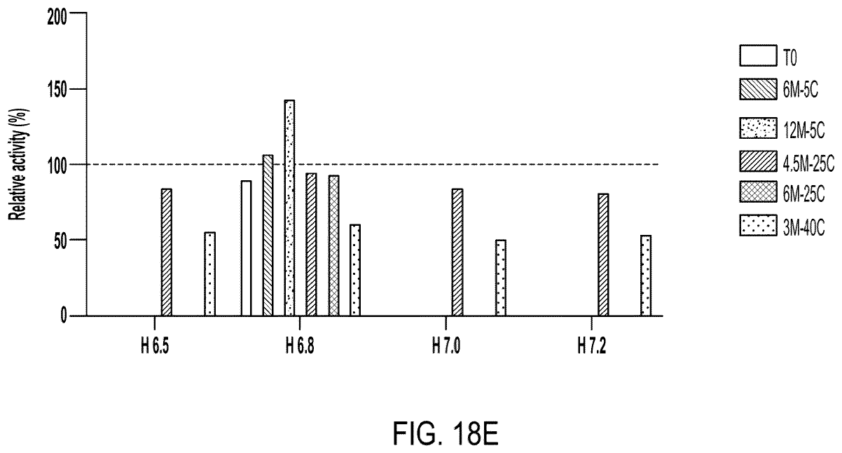

As shown in FIG. 18, a general increase in the level of hotspot modifications as a function of temperature and time was observed in all buffers and pH values tested. Specifically, for D32 isomerization, a modification level of approximately 6% per week was observed in the histidine buffer formulation at pH 6.8 upon storage for 3 months at 40° C., while at 5° C. a modification level of approximately 3% was observed upon storage for 12 months. No significant differences in modification levels were observed between different pH values and buffer species (FIG. 18A-D). At 2 months of storage at 40° C. (2M-40C), K7.0 buffer showed an out-of-trend extent of HC M251 oxidation, likely due to operator error. Biacore analysis indicated that SAR445088 retained relative activity after 6 months storage at 25° C. and 12 months at 5° C. for samples in histidine buffer at pH 6.8. A slight decrease in potency was observed for samples exposed to 40° C. for 3 months.

### Summary

SAR445088 formulations demonstrated a higher tendency to form HMW species in potassium phosphate buffer than in histidine buffer. Potassium phosphate buffer had lower levels of PS80 degradation compared to histidine buffer. However, since effective mitigation of PS80 degradation in the presence of histidine was observed with 10 μM DTPA, the propensity of the molecule to aggregate was a primary driver for selecting histidine as the buffer. Critical quality attributes (CQAs) of the formulations did not significantly differ across a pH range of 6.7-7.2 in histidine buffer. Apart from a relative increase in 2 μm particle formation in OMPI PFSs, CQAs did not otherwise significantly differ when comparing BD Neopak Low, BD Neopak High and OMPI syringes. OMPI syringes also had a high ≤10 μm and ≤25 μm sub-visible particle count but the particle counts were below USP specification limits (<6000 10 μm particles per small volume container and <600 25 μm particles per small volume container).

### Batch 2 Formulation Study

### PFS Stability Study

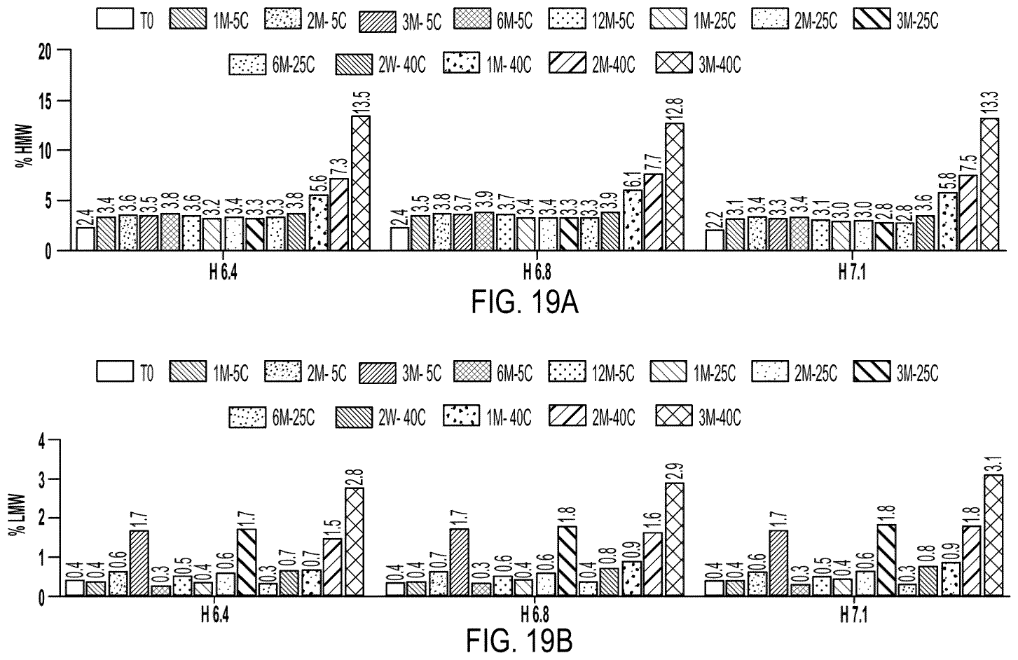

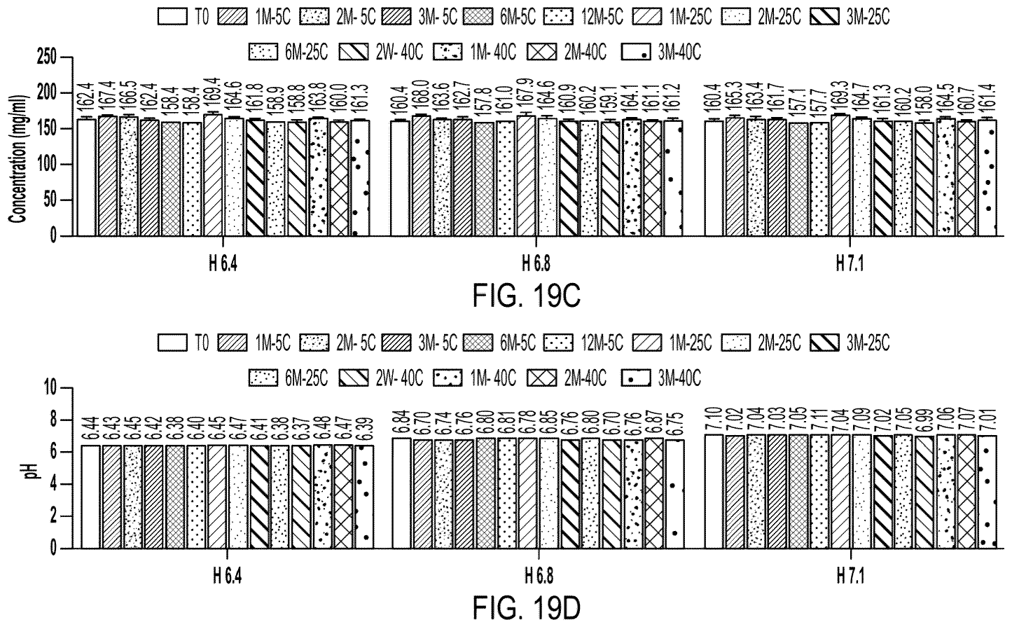

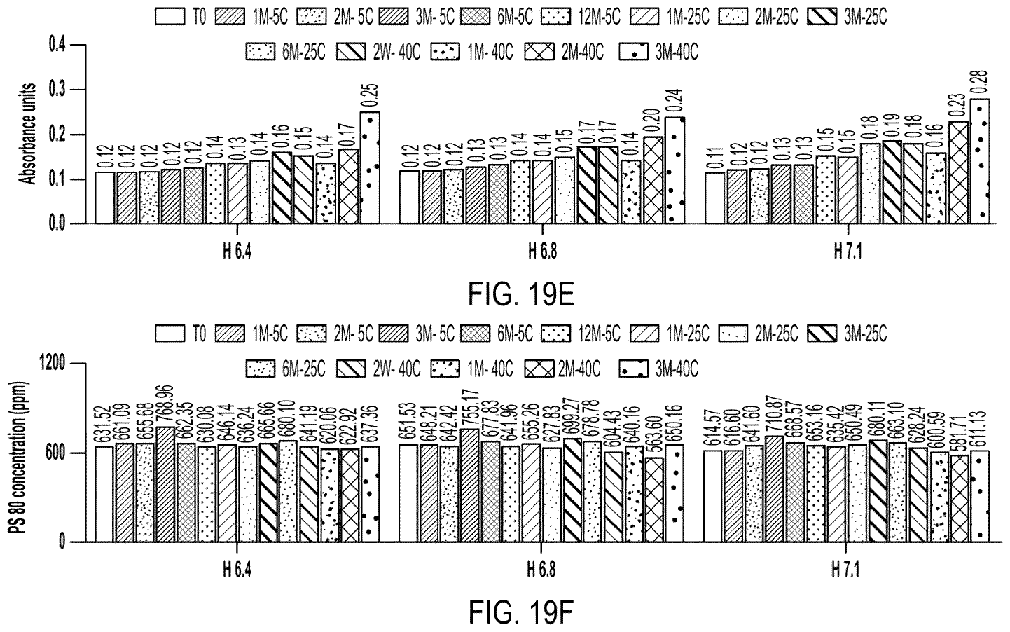

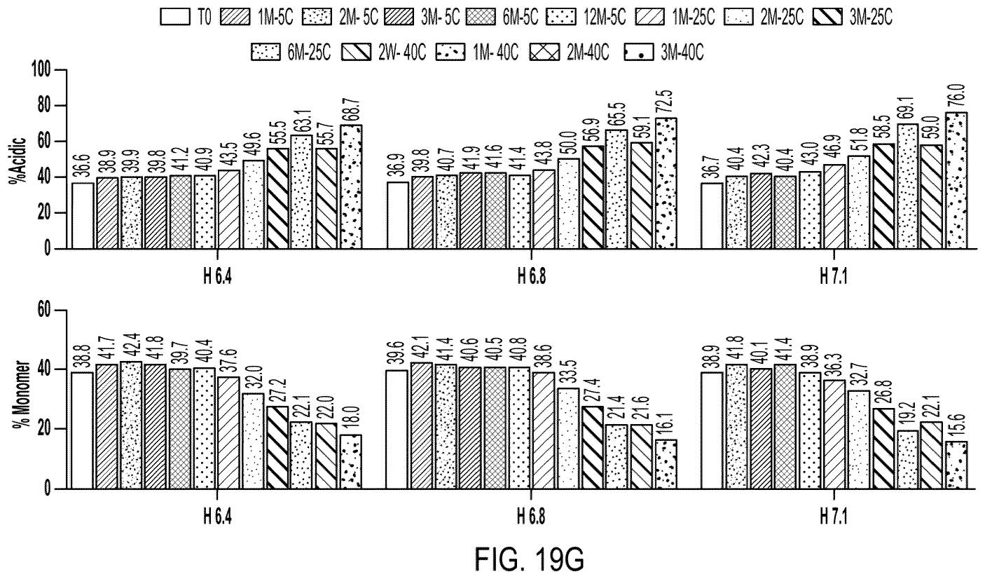

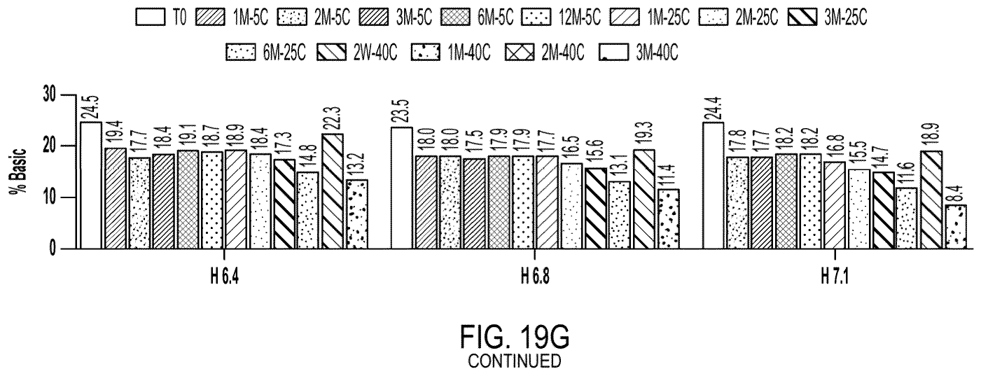

As shown in FIG. 19, an overall increase in HMW species formation over time and at 40° C. was observed. No significant change in level of HMW species formation occurred at 5° C. or 25° C. between pH 6.4-7.1. Slightly lower HMW species formation at T0 and subsequent timepoints at 5° C. and 25° C. was observed for H7.1 compared to H6.4 and H6.8 formulations (FIG. 19A). An increase in LMW species formation with time and at 40° C. was observed. No trends in LMW species formation were observed as a function of formulation pH at 5° C. and 25° C. (FIG. 19B). No effect of formulation pH was seen on antibody concentration (FIG. 19C) and pH at the end of the storage period (FIG. 19D). A general increase in turbidity with time and temperature was observed for all the tested formulations, but no significant differences were observed between different pHs (FIG. 19E). Negligible PS80 degradation was observed in any of the test formulations under all storage stability conditions (FIG. 19F). Charged variant analysis indicated an overall increase in occurrence of acidic variants and a corresponding decrease in monomer and basic variants at 25° C. and 40° C. for all the formulations tested. Due to loss of peak resolution, charged variant analysis was only performed at 40° C. up to 1 month of storage. Charged variants remained relatively consistent at 5° C. for all the conditions tested (FIG. 19G).

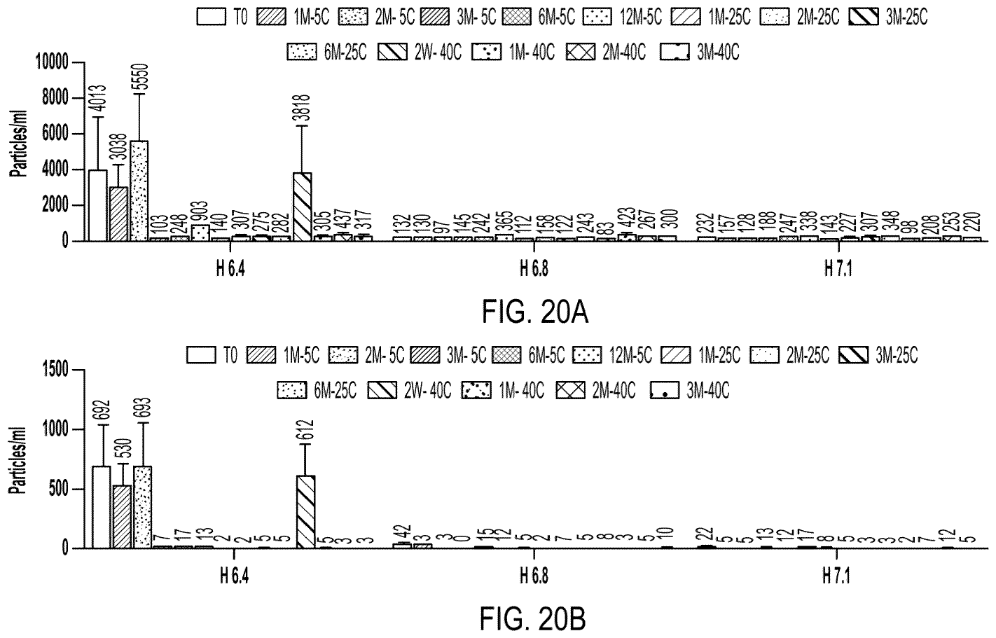

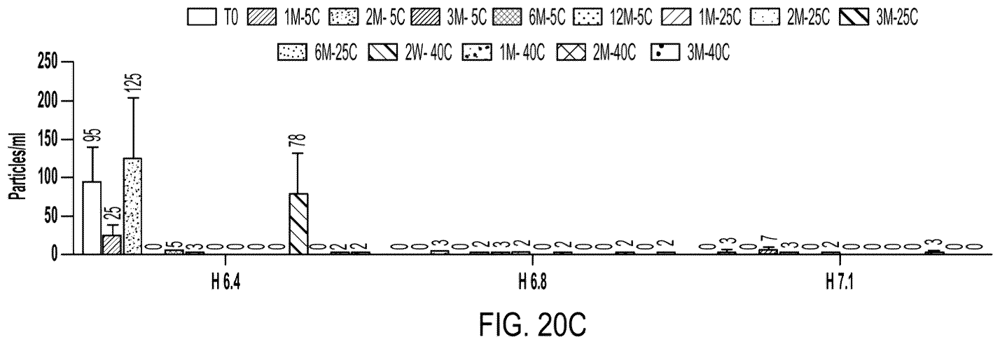

As shown in FIG. 20, 2 μm subvisible particles were more abundant than larger particles in all the tested formulations, presumably due to the presence of silicone oil. However, no notable increase in occurrence of sub-visible particles with storage at different temperatures was apparent. Appearance of sub-visible particles≤2 μm, ≤10 μm and ≤25 μm in size was largely consistent between formulation test samples, with a couple of outliers attributed to assay variability (FIGS. 20A-C).

### Vial Stability Study

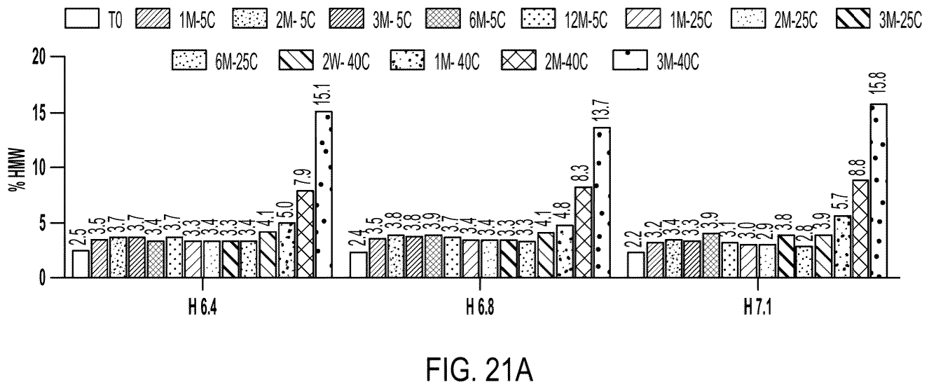

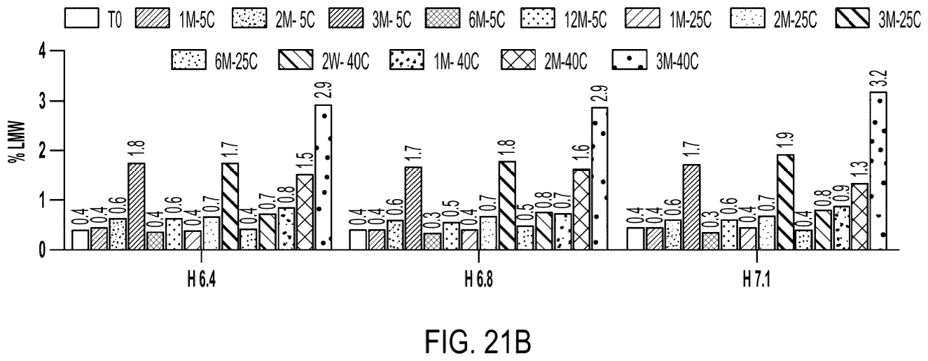

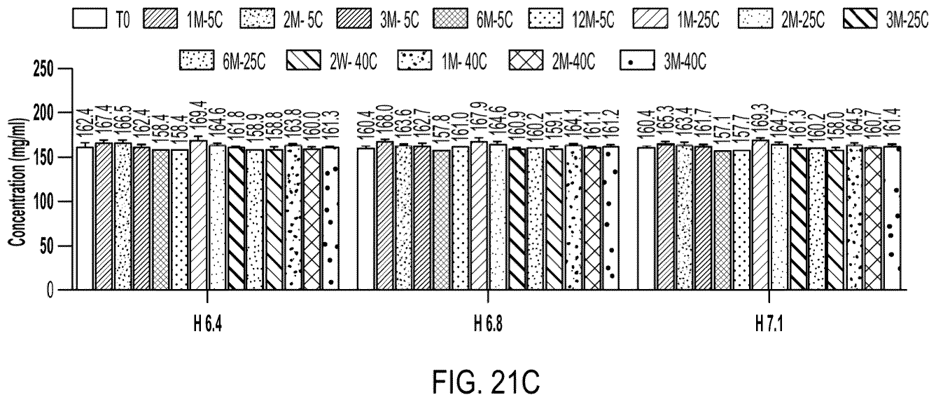

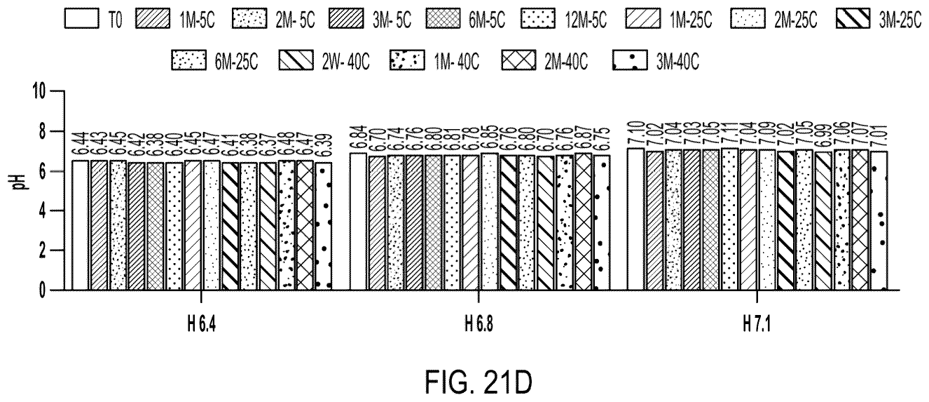

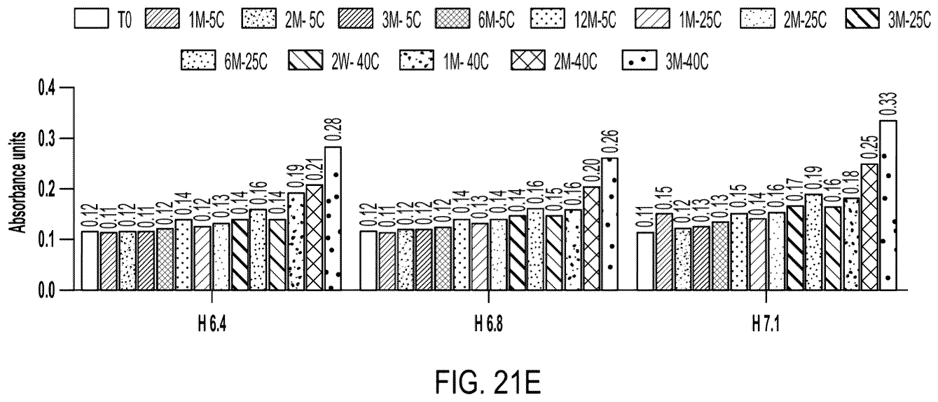

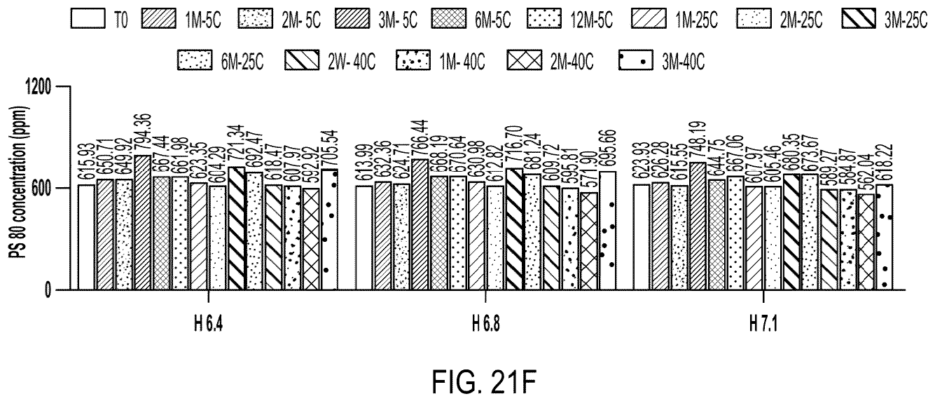

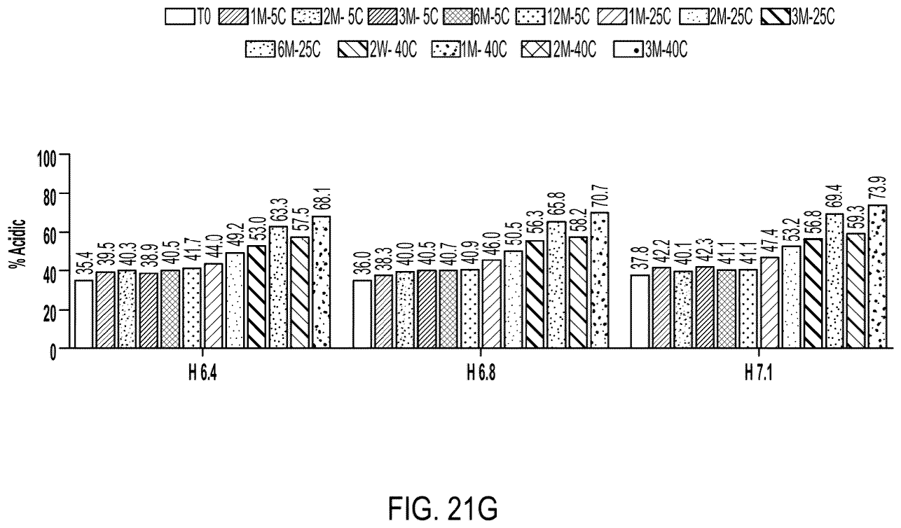

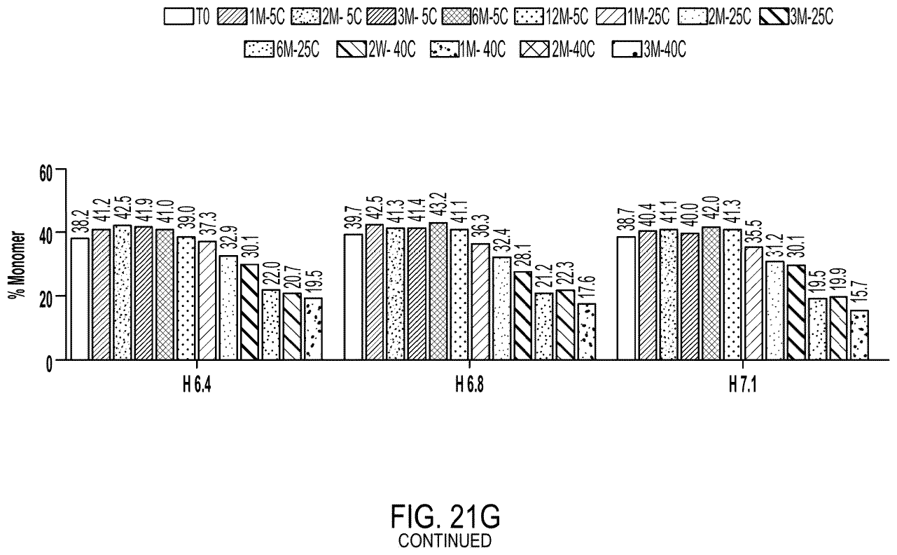

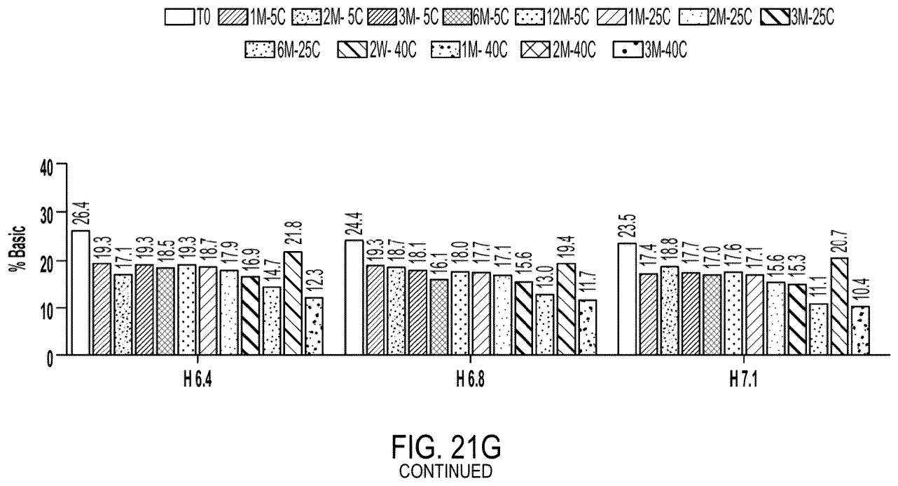

As shown in FIG. 21, an overall increase in HMW species formation over time and at 40° C. was observed. No significant change in HMW species formation occurred at 5° C. or 25° C. between pH 6.4-7.1. Slightly lower HMW species formation at T0 and subsequent timepoints at 5° C. and 25° C. was observed for H7.1 compared to H6.4 and H6.8 formulations (FIG. 21A). An increase in LMW species formation with time and at 40° C. was observed. No trends in LMW species formation were observed as a function of formulation pH at 5° C. and 25° C. (FIG. 21B). No effect of formulation pH was seen on antibody concentration (FIG. 21C) and pH at the end of the storage period (FIG. 21D). A general increase in turbidity with time and temperature was observed for all the tested formulations, but no significant differences were observed between different pHs (FIG. 21E). No major PS80 degradation was observed in any of the test formulations under all storage stability conditions (FIG. 21F). Charged variant analysis by cIEF indicated an overall increase in occurrence of acidic variants and a corresponding decrease in monomer and basic variants at 25° C. and 40° C. for all the formulations tested. Due to loss of peak resolution, charged variant analysis was only performed at 40° C. up to 1 month of storage. Charged variants remained relatively consistent at 5° C. for all the conditions tested (FIG. 21G).

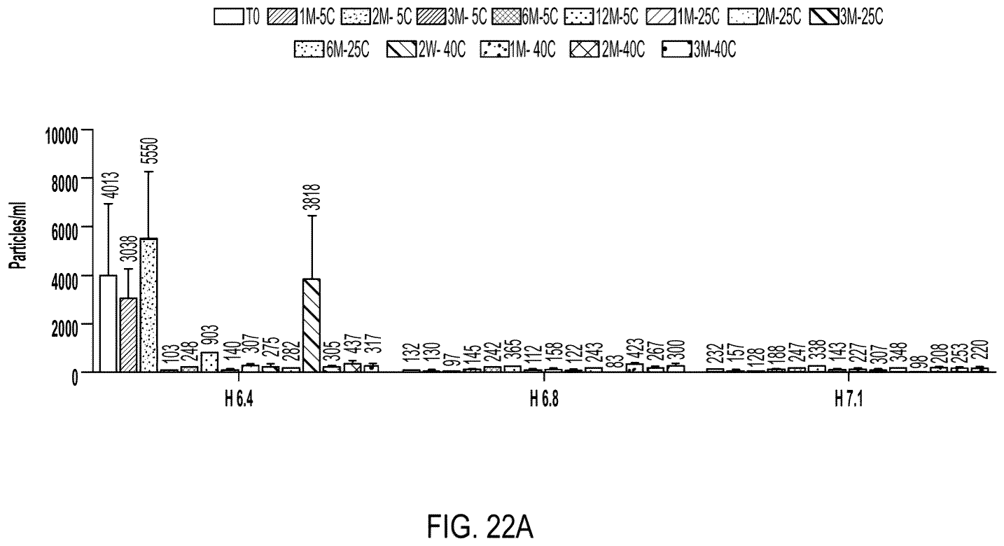

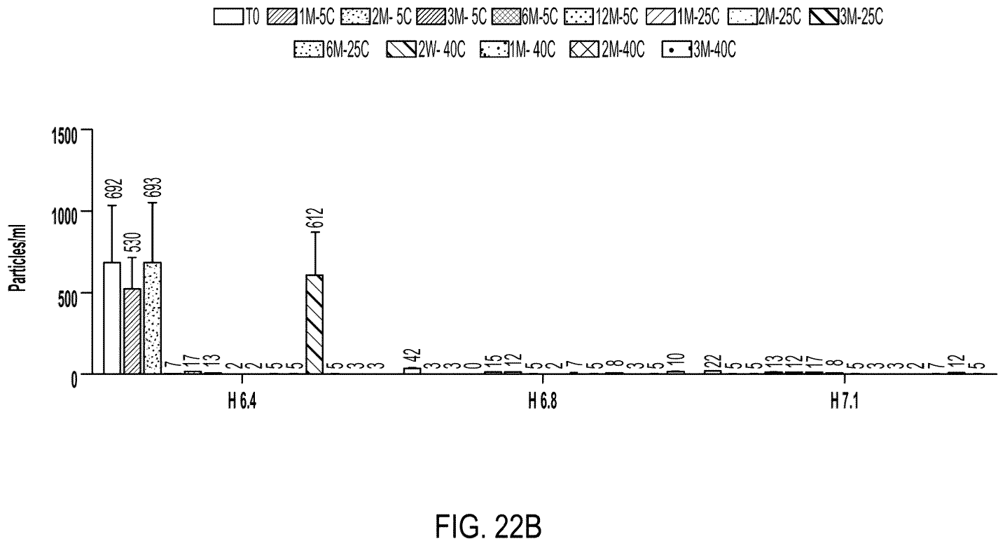

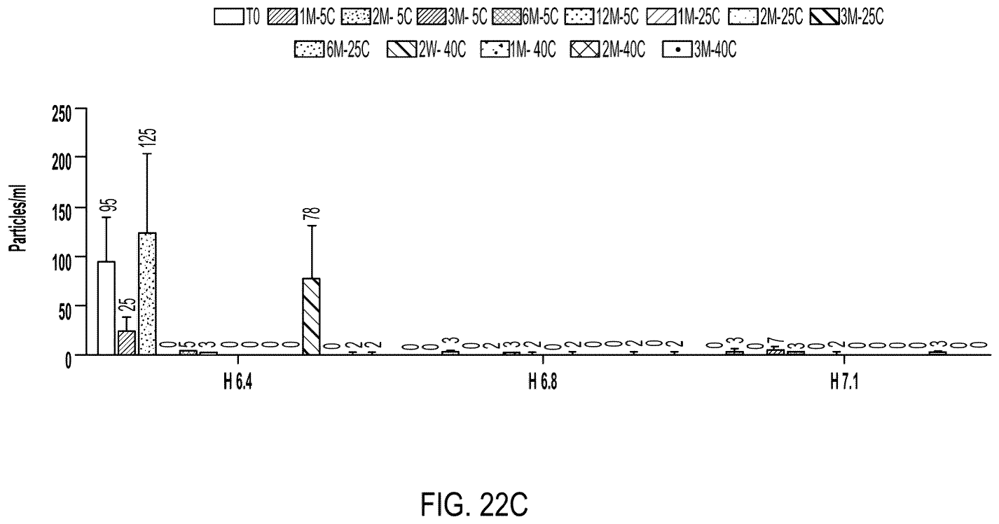

As shown in FIG. 22, no notable increase in formation of sub-visible particles with storage at different temperatures was apparent in any of the tested formulations. Appearance of sub-visible particles≤2 μm, ≤10 μm and ≤25 μm in size was largely consistent between formulation test samples, with a couple of outliers attributed to assay variability (FIGS. 22A-C).

### Frozen-Storage Stability Study

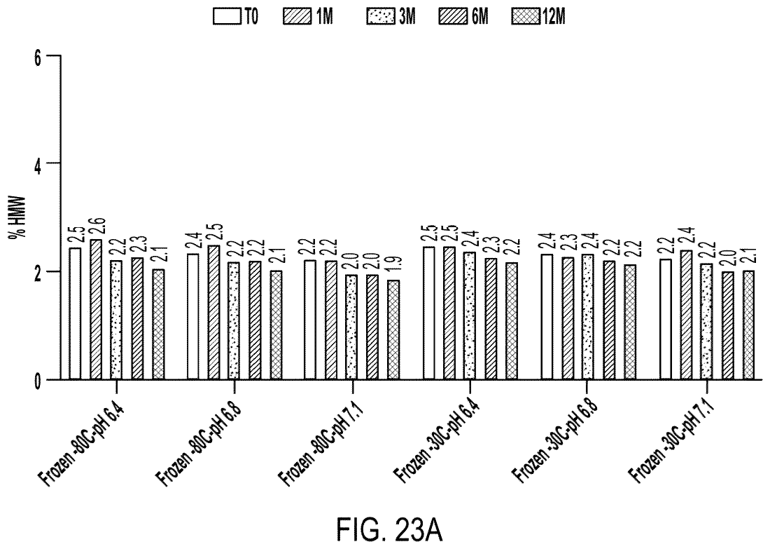

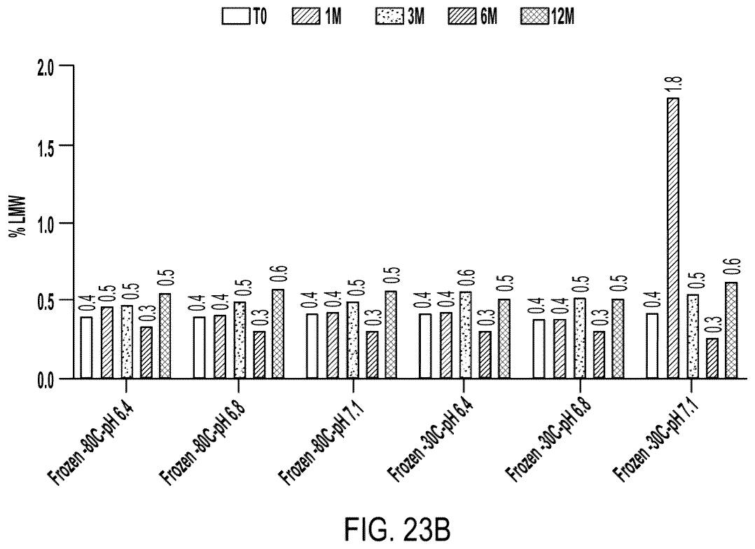

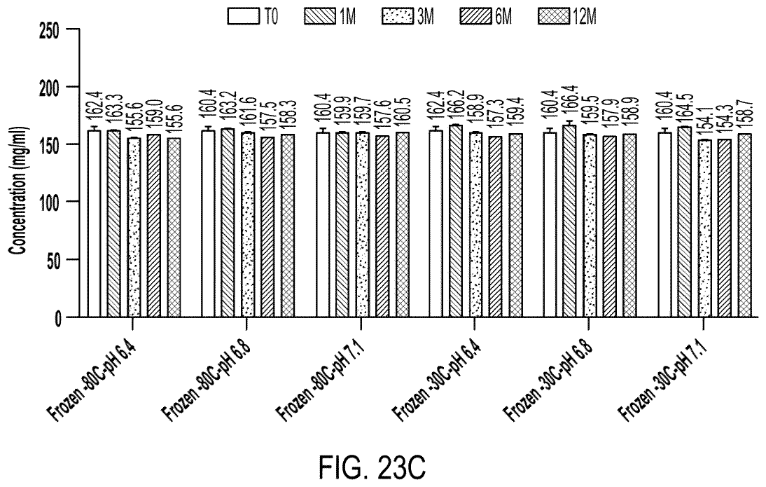

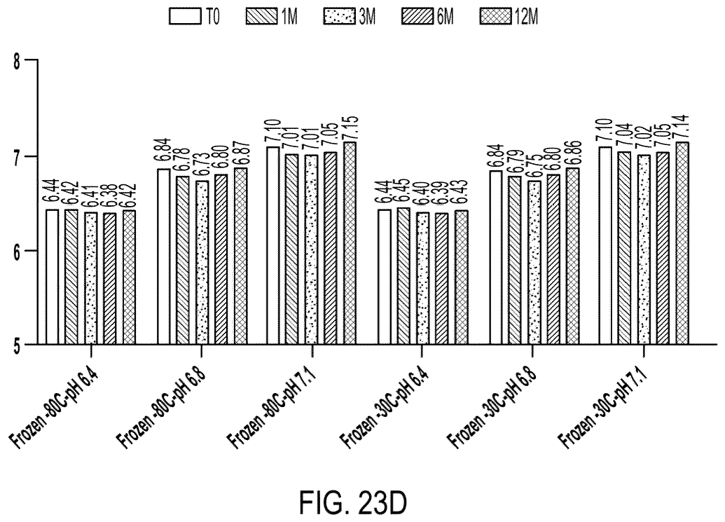

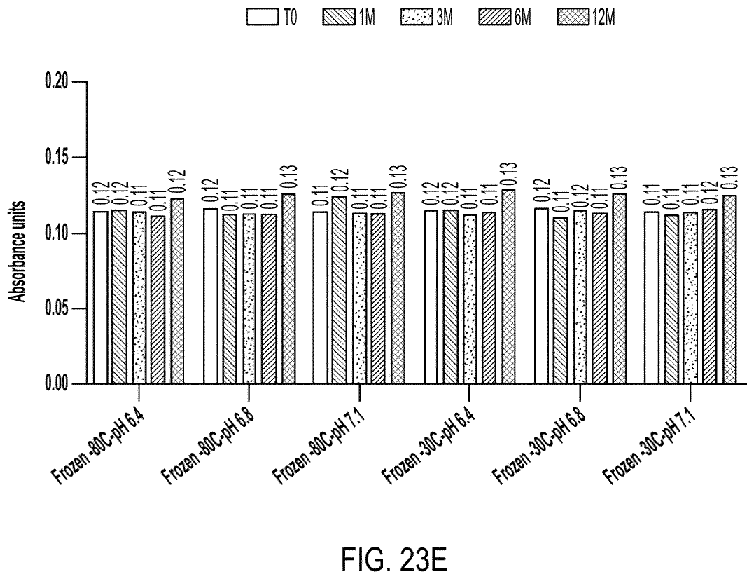

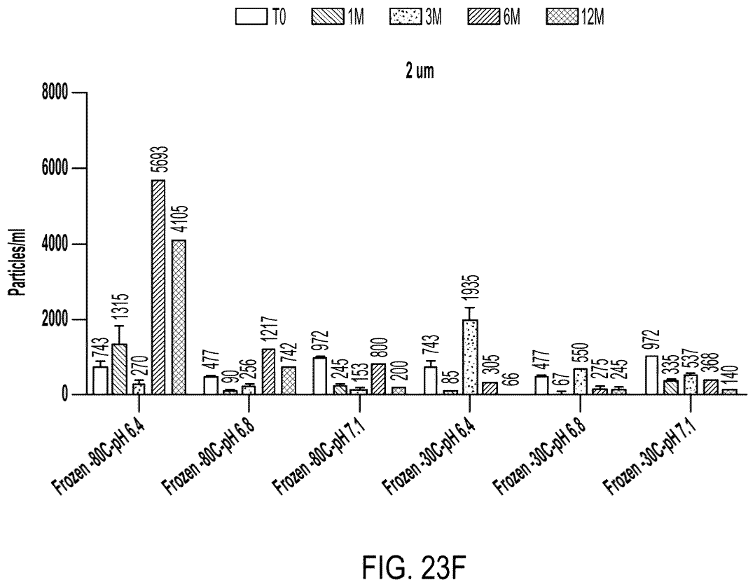

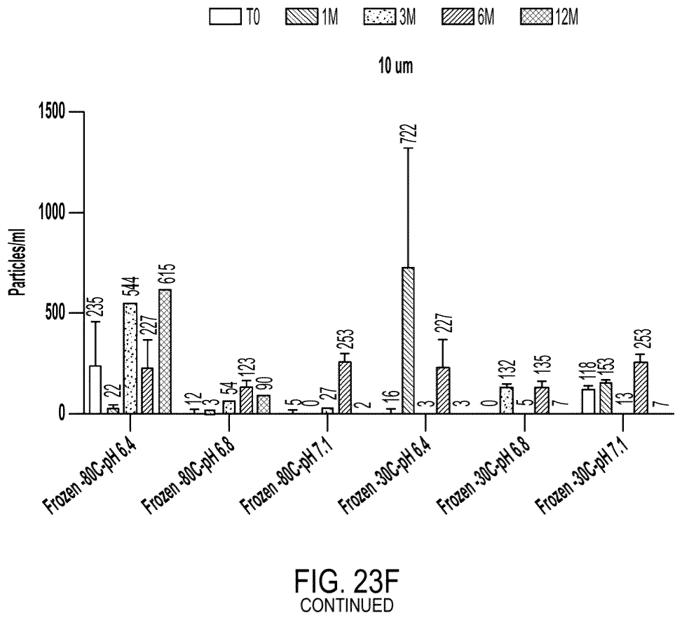

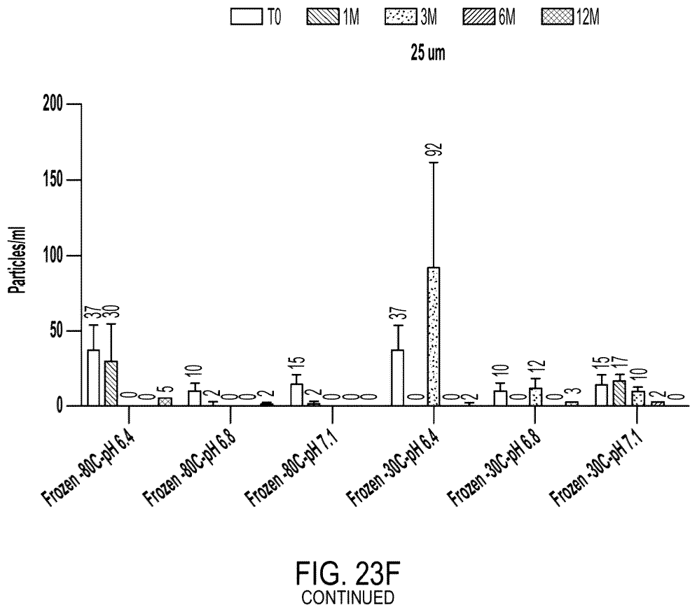

As shown in FIG. 23, there were no major changes in HMW species formation upon frozen storage at −80° C. and −30° C. across the different formulations tested (FIG. 23A). LMW species formation remained similar over time upon frozen storage at −80° C. and −30° C. The formulation sample with histidine buffer at pH 7.1 (H7.1) stored at −30° C. showed out-of-trend level of LMW species formation. This was attributed to assay variability (FIG. 23B). Frozen storage had no apparent effect on antibody concentration (FIG. 23C), pH (FIG. 23D), turbidity (FIG. 23E) or sub-visible particle formation level at the end of the storage period (FIG. 23F).

### Peptide Map and Biacore Analysis

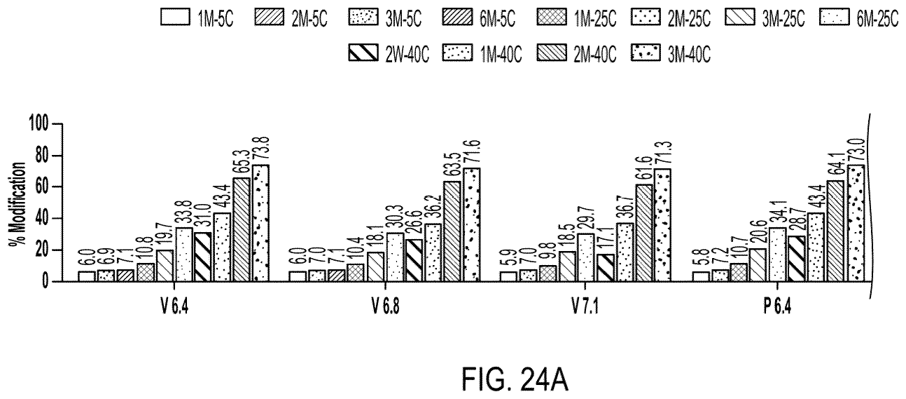

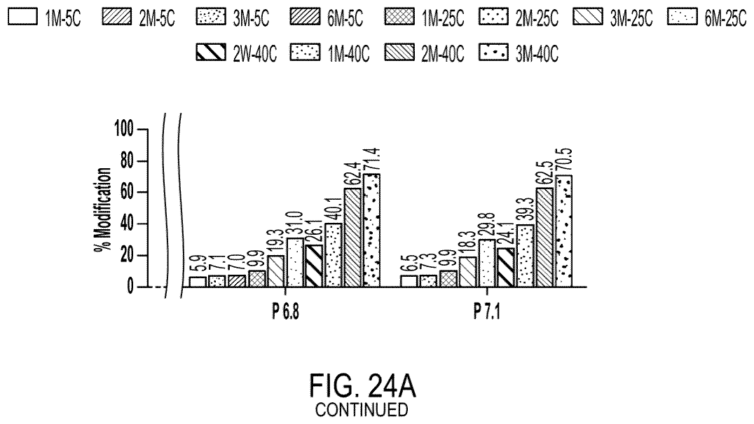

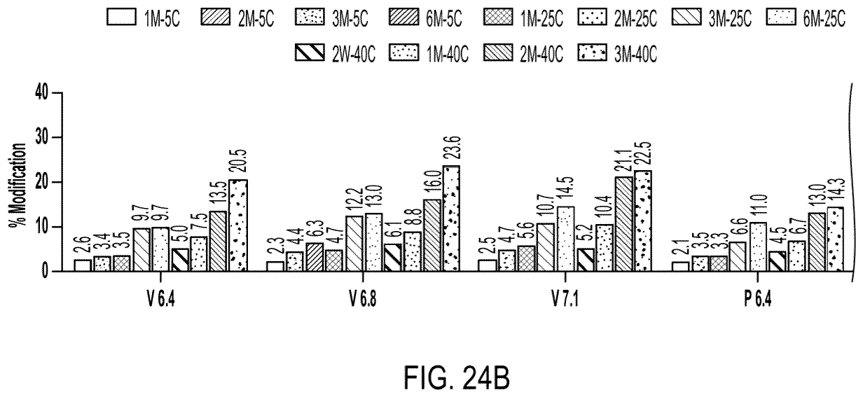

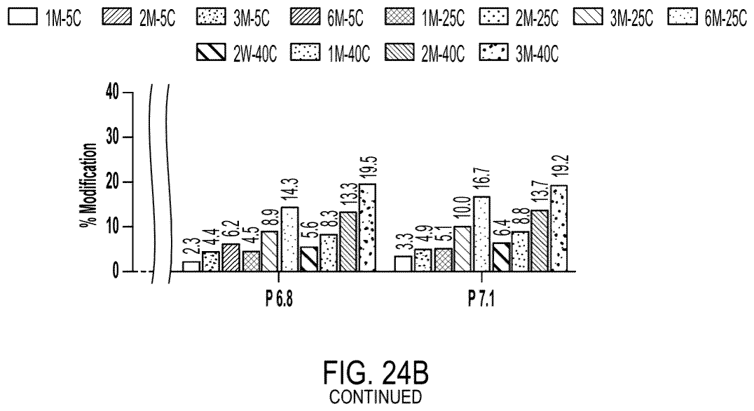

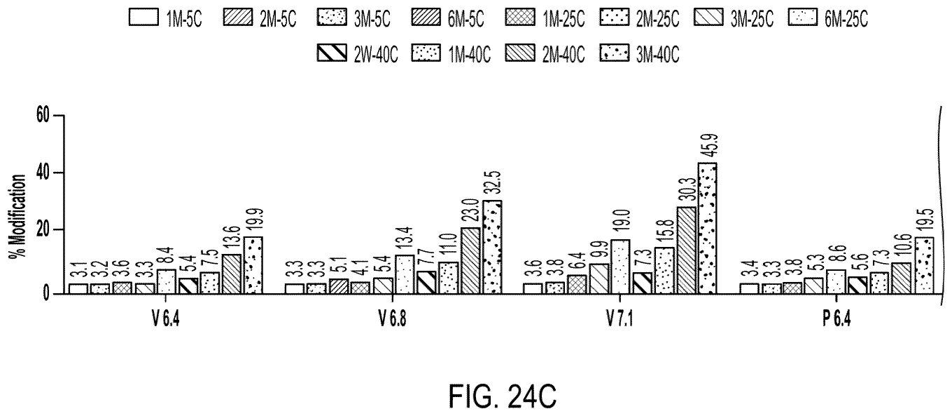

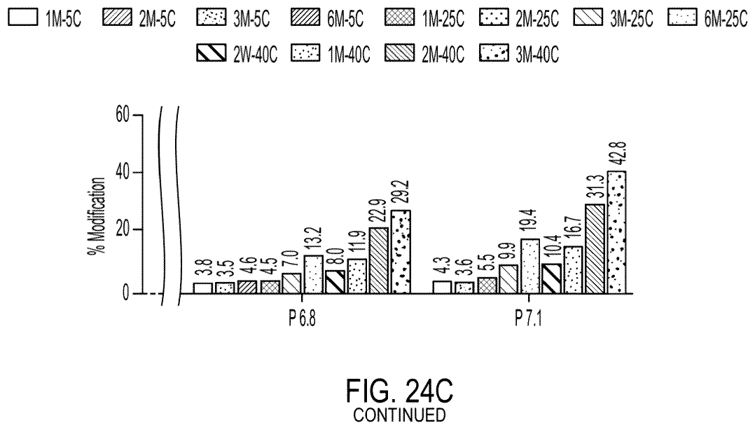

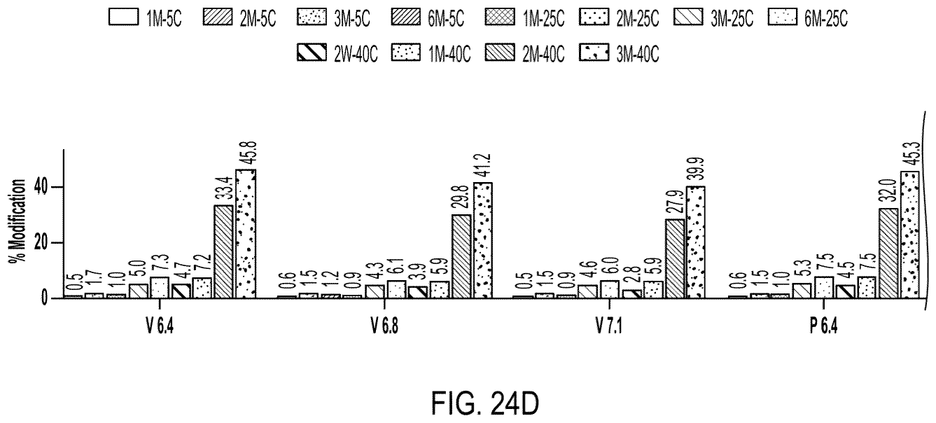

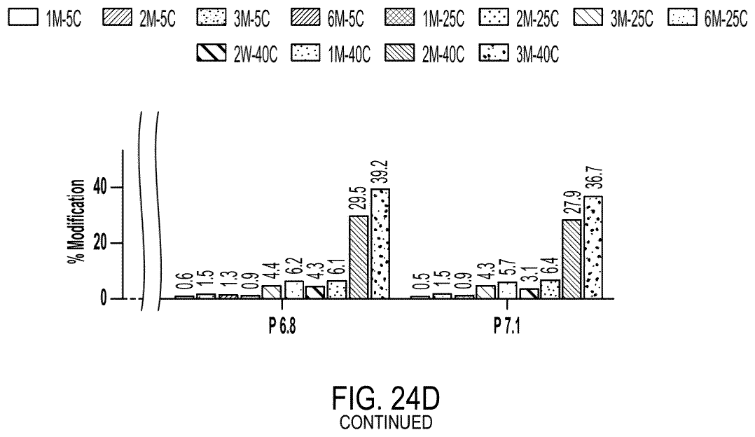

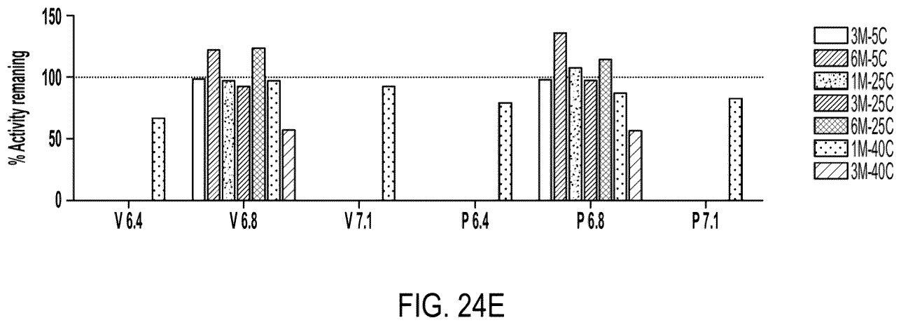

As shown in FIG. 24, a general increase in the level of hotspot modifications as a function of temperature and time was observed in all buffers and pHs tested, in vials and in PFS. Specifically, for D32 isomerization, at 40° C., a modification level of approximately 6% per week was observed in the histidine buffer formulation at pH 6.8 (H6.8). No significant differences in modification levels were observed between different pH values and buffer species (FIGS. 24A-D). Biacore analysis indicated that SAR445088 retained relative activity after 6 months storage at 25° C. and 5° C. for samples in histidine buffer at pH 6.8. A slight decrease in potency was observed for all samples exposed to 40° C. for 3 months (FIG. 24E).

### Summary

SAR45088 test formulations having pHs between 6.4 and 7.1 showed no significant differences for all the CQA's tested. Formulation samples placed in PFSs and vials and subjected to frozen stability maintained substantial stability over the course of the study (12 months).

### Batch 3 Formulation Study

### PFS Stability Study

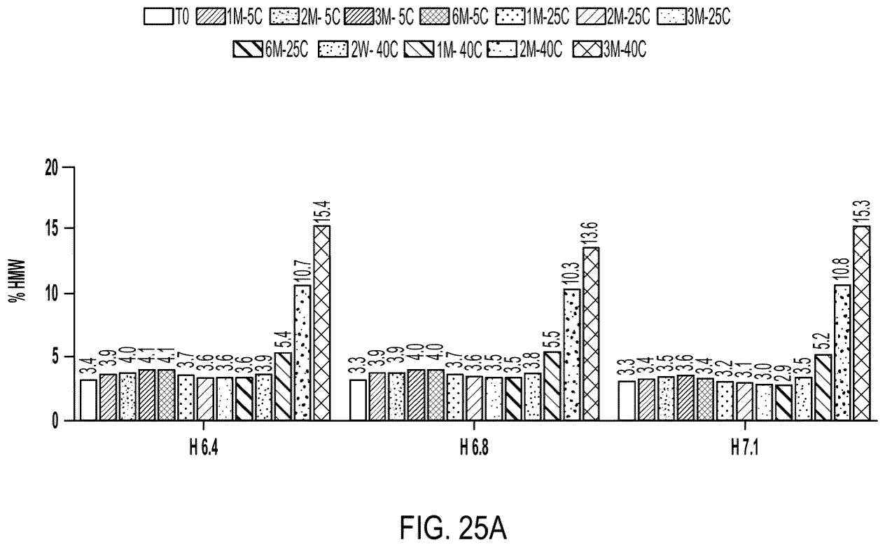

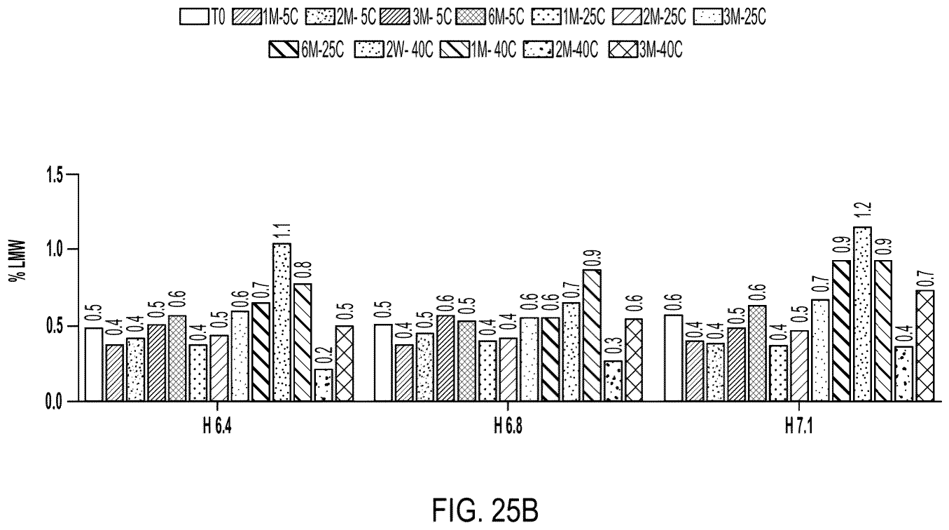

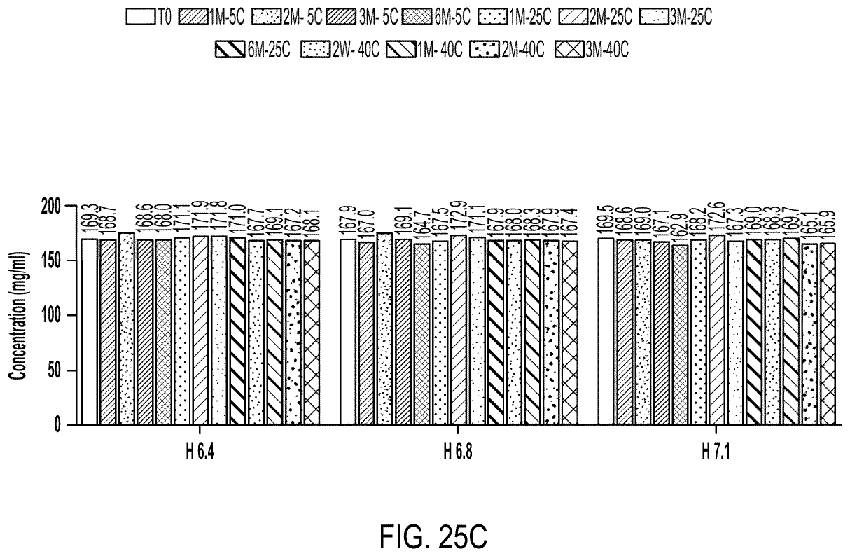

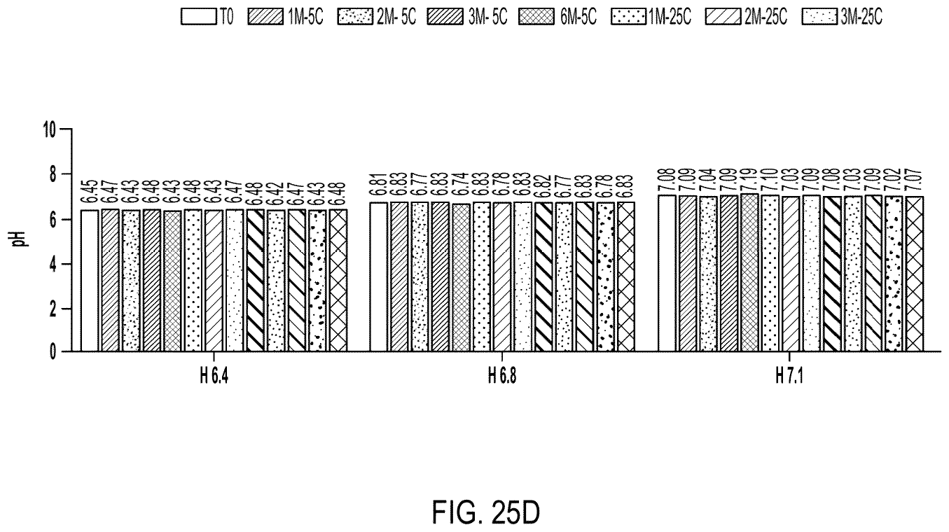

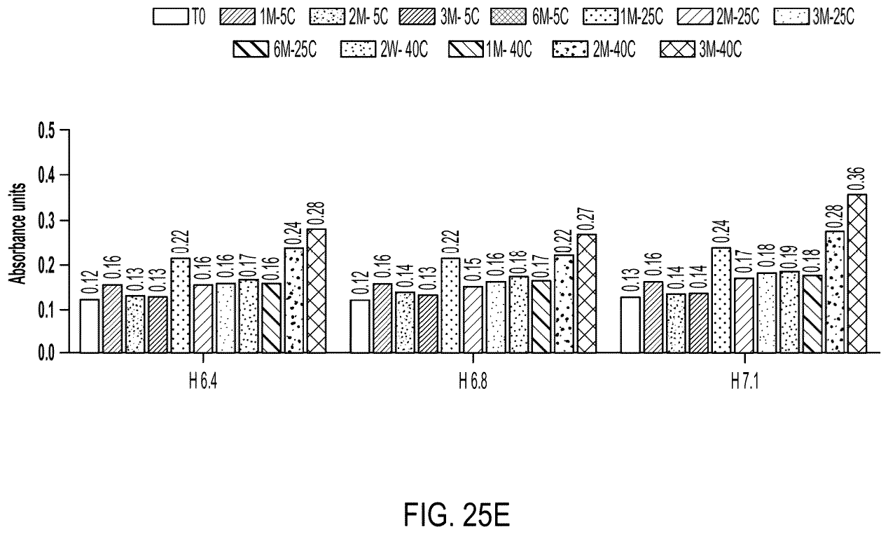

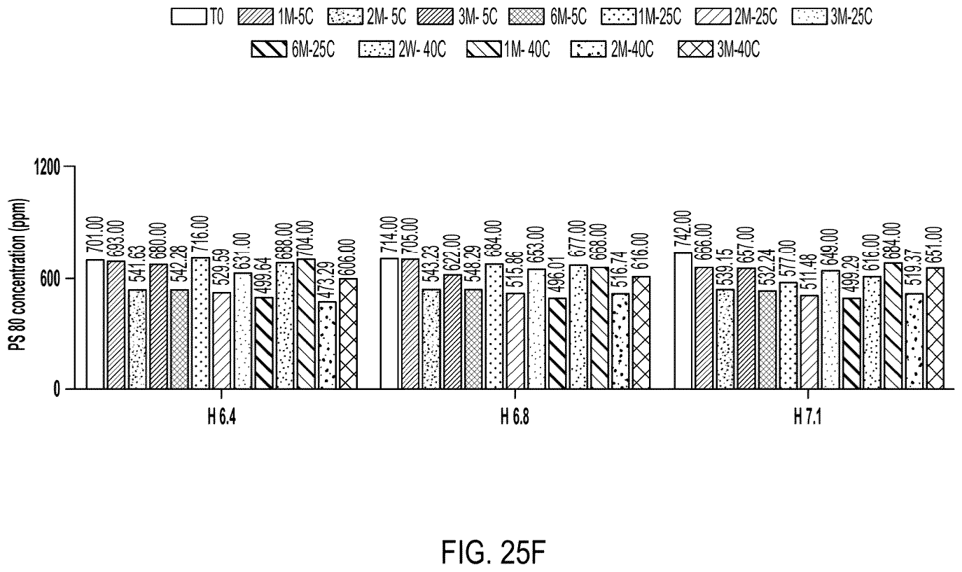

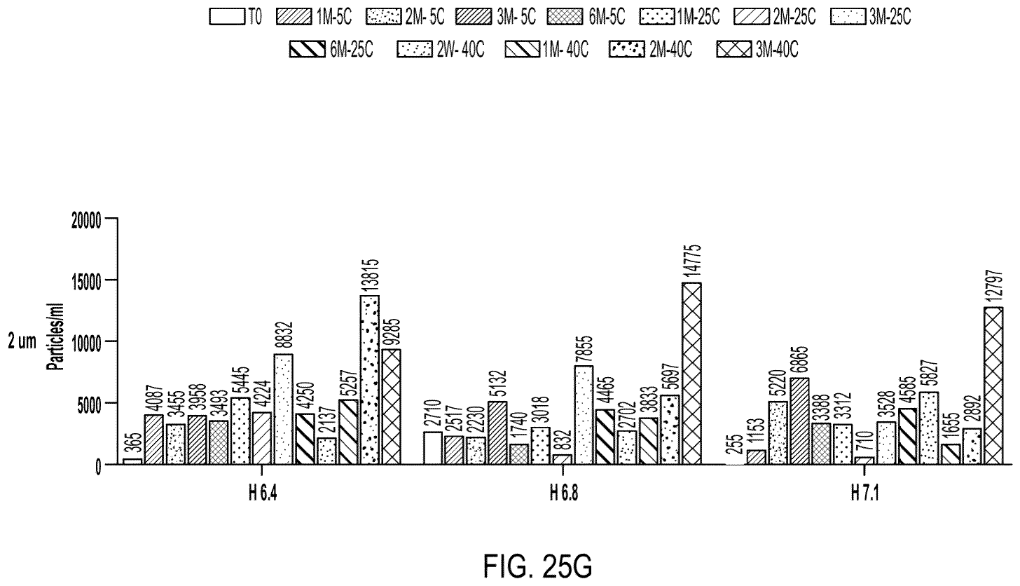

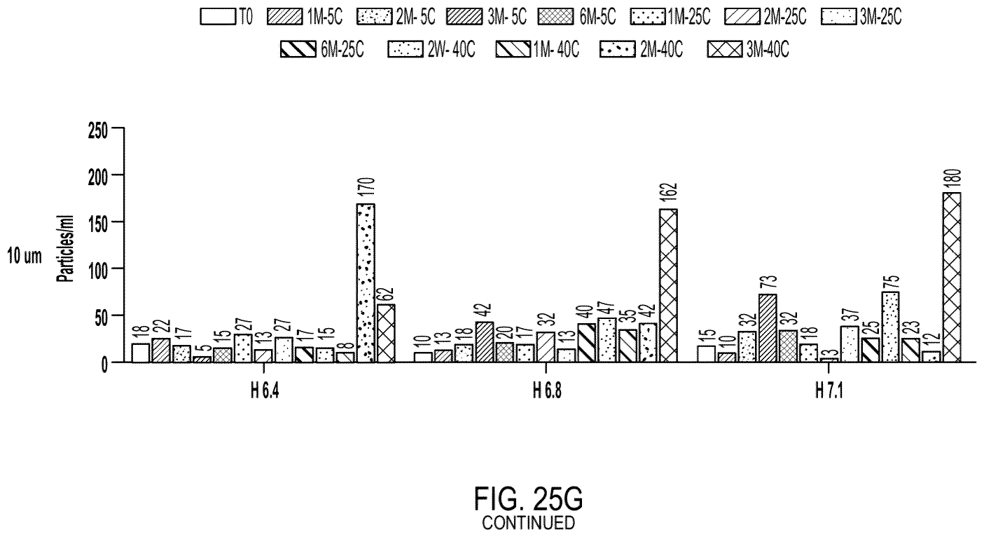

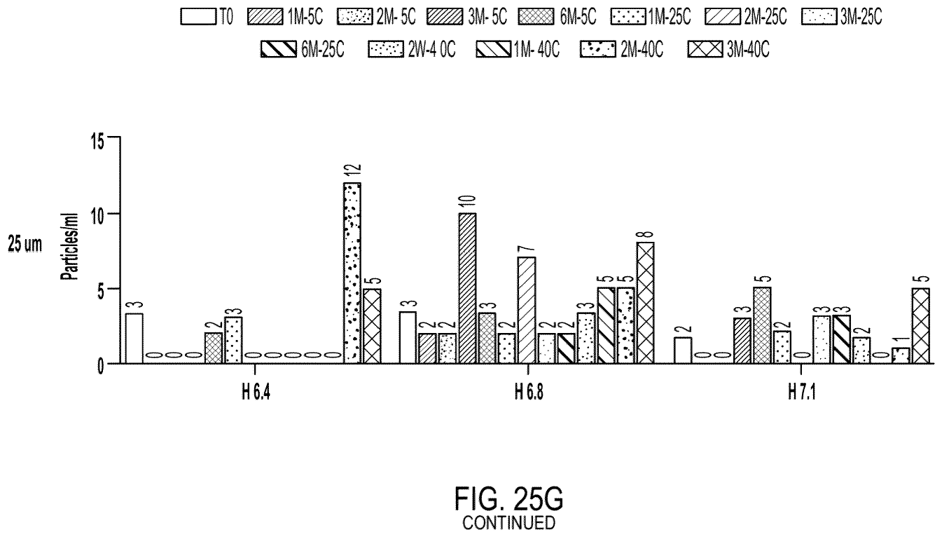

As shown in FIG. 25, an overall increase in HMW species formation over time and at 40° C. was observed. No significant change in level of HMW species formation was observed at 5° C. or 25° C. between pH 6.4-7.1. Slightly lower HMW species formation at T0 and subsequent timepoints at 5° C. and 25° C. was observed for H7.1 compared to H6.4 and H6.8 formulations (FIG. 25A). No distinct differences were observed in levels of LMW species formation between the test pHs (FIG. 25B). Formulation pH did not have an observable effect on concentration (FIG. 25C) or pH (FIG. 25D) measured at the end of the storage period. A general increase in turbidity with time and temperature was observed for all test formulations, but no significant differences were observed between different pHs (FIG. 25E). Negligible PS80 degradation was observed in any test formulation under all storage stability conditions (FIG. 25F). Lastly, all test formulations had a higher number of 2 μm subvisible particles than particles of other size. However, there was no obvious increase in sub-visible particle levels upon storage at 5° C. and 25° C. An increase in sub-visible particles was observed in all formulations over time at 40° C. (FIG. 25G). There were no major differences in sub-visible particle trends across the pH values studied.

### Vial Stability Study

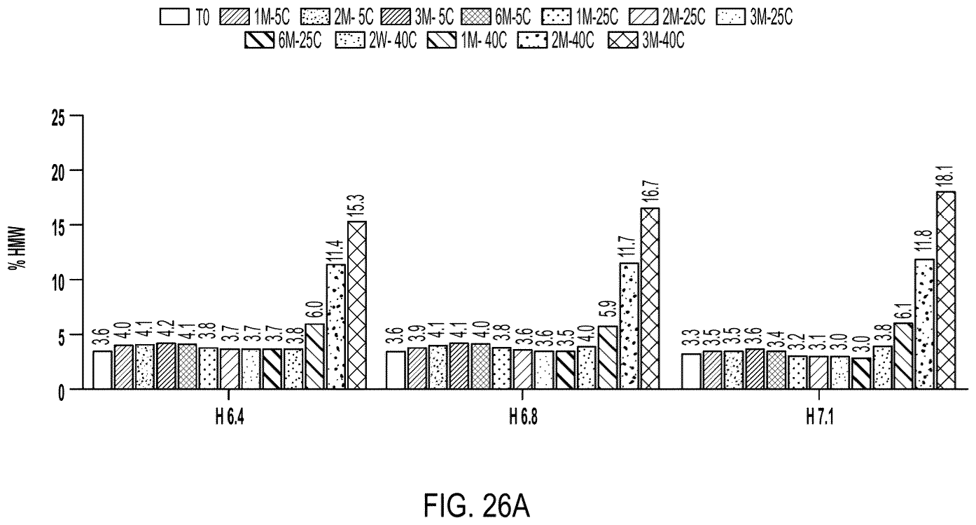

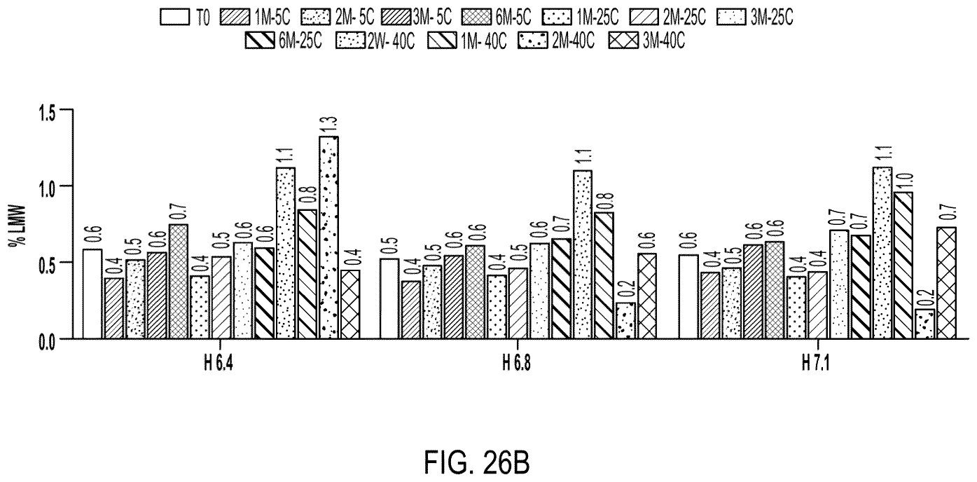

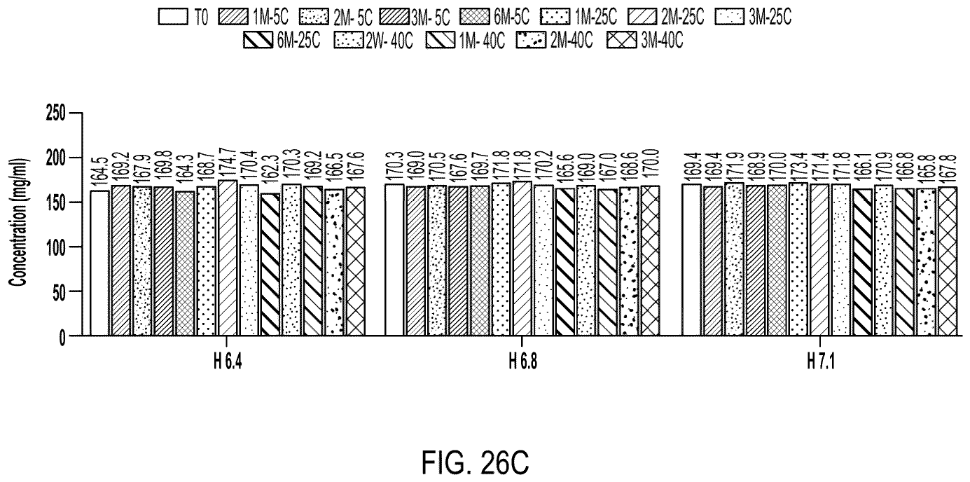

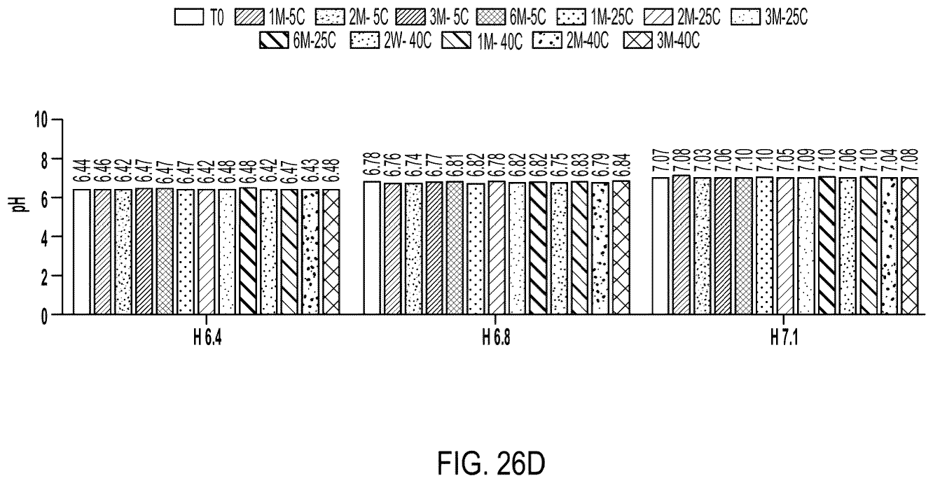

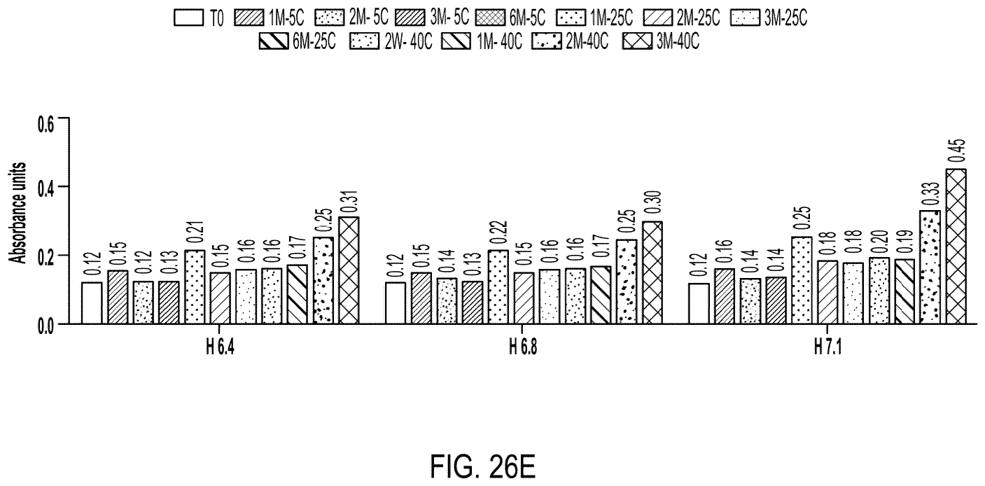

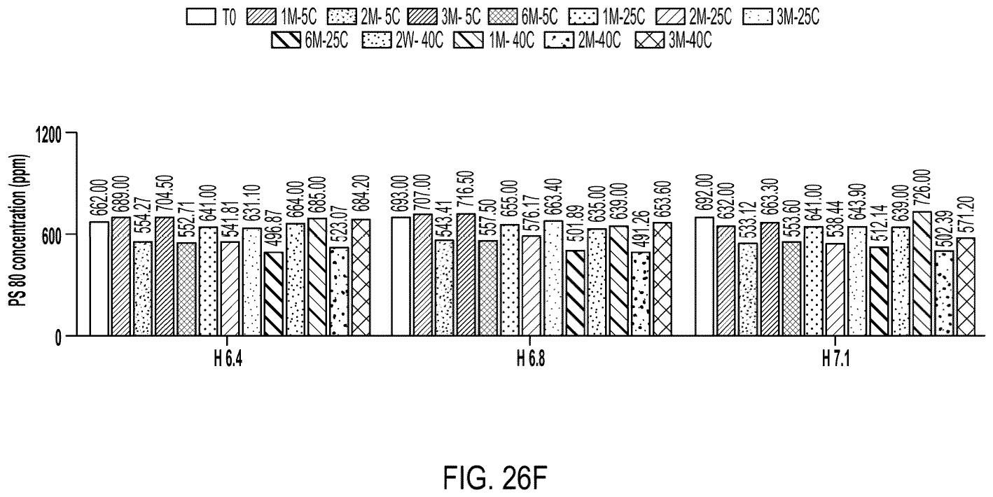

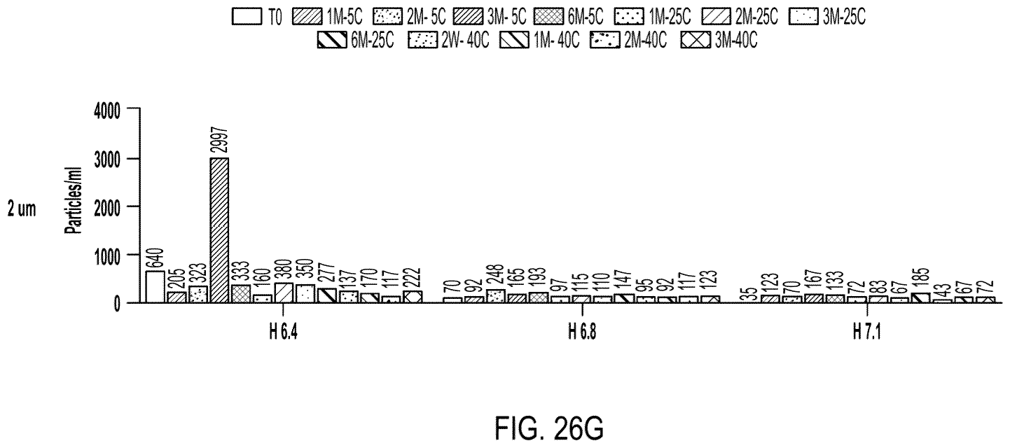

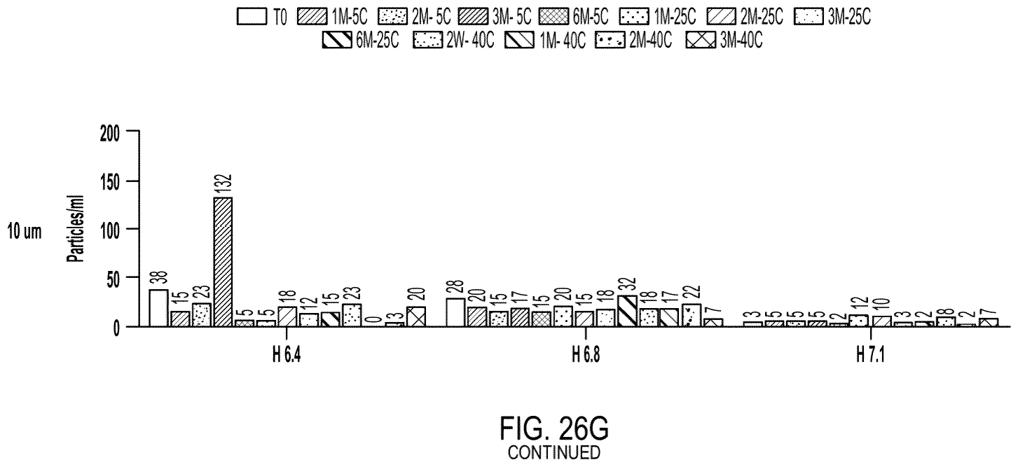

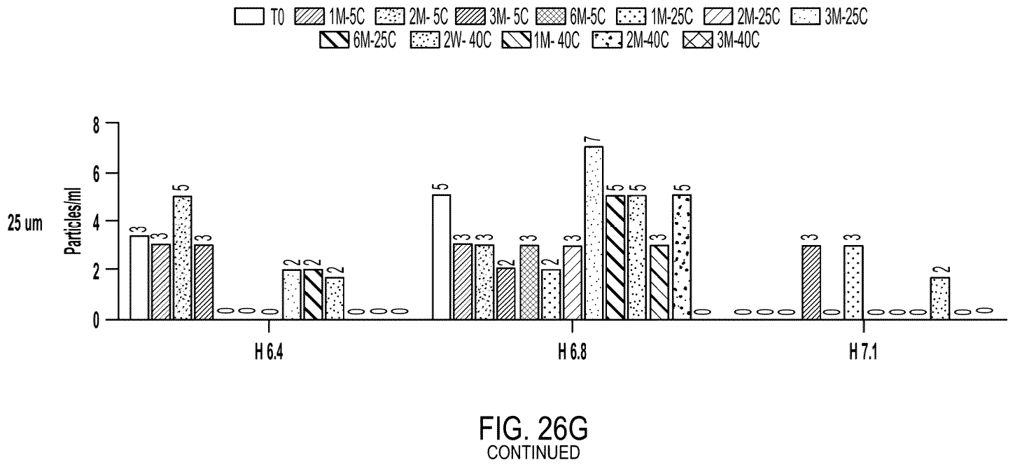

As shown in FIG. 26, an overall increase in HMW species formation over time and at 40° C. was observed. No significant change in level of HMW species formation was observed at 5° C. or 25° C. between pH 6.4-7.1. Slightly lower HMW species formation at T0 and subsequent timepoints at 5° C. and 25° C. was observed for H7.1 compared to H6.4 and H6.8 formulations (FIG. 26A). No distinct differences were observed in levels of LMW species formation between the test pHs (FIG. 26B). Formulation pH did not have an observable effect on concentration (FIG. 26C) or pH (FIG. 26D) measured at the end of the storage period. A general increase in turbidity with time and temperature was observed for all test formulations, but no significant differences were observed between different pHs (FIG. 26E). Negligible PS80 degradation was observed in any test formulation under all storage stability conditions (FIG. 26F). Lastly, there was no obvious increase in the level of sub-visible particle formation upon storage at different temperatures in any of the test formulations. Level of formation of sub-visible particles≤2 μm, ≤10 μm and ≤25 μm in size was largely consistent, with a couple of outliers attributed to operator error (FIG. 26G).

### Peptide Map and Biacore Analysis

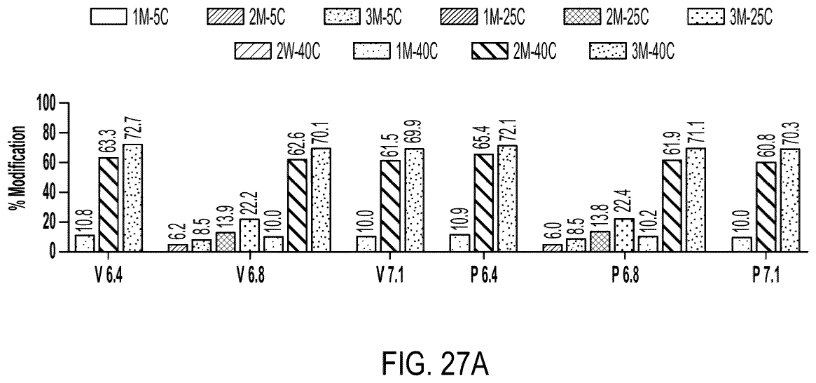

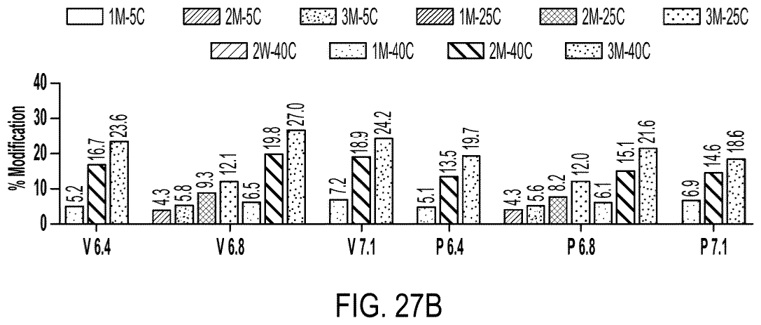

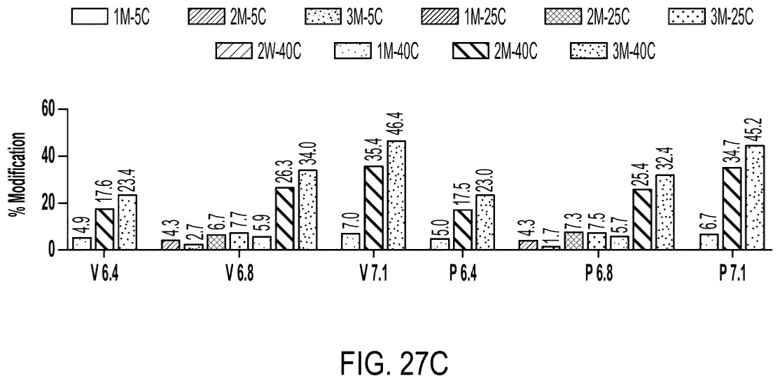

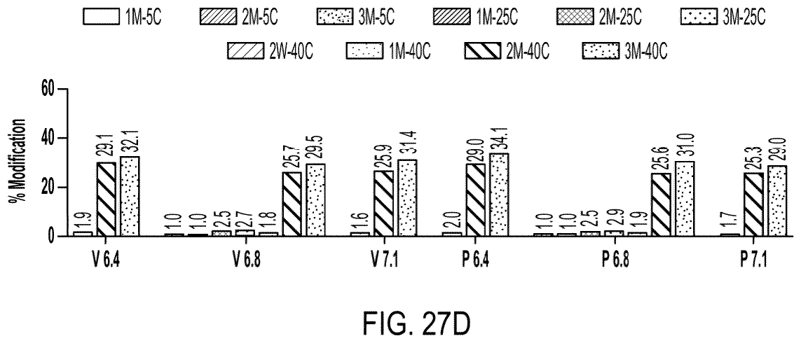

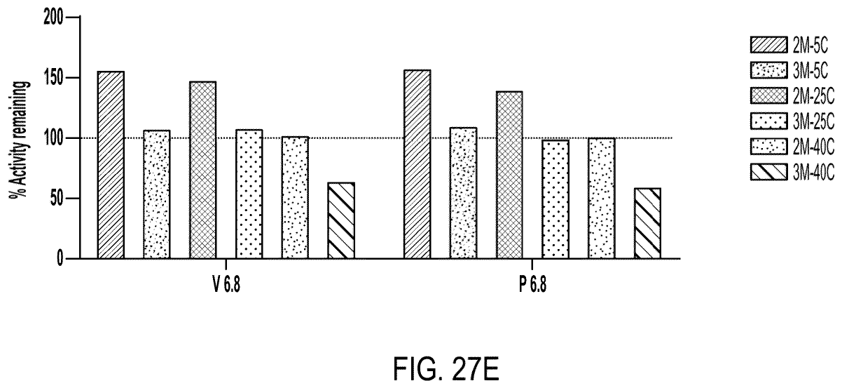

As shown in FIG. 27, a general increase in the level of hotspot modifications as a function of temperature and time was observed in all buffers and pHs tested. Specifically, for D32 isomerization, at 40° C., a modification level of approximately 6% per week was observed in the histidine buffer formulation at pH 6.8 (H6.8). No significant differences in modification levels were observed between different pH values and buffer species (FIG. 27A-D). Biacore analysis indicated that SAR445088 retained relative activity after 3 months storage at 25° C. and 5° C. for samples in histidine buffer at pH 6.8. A slight decrease in potency was observed for all samples exposed to 40° C. for 3 months (FIG. 27E).

### Summary

SAR445088 test formulations having pHs between 6.4 and 7.1 showed no significant differences for all the CQA's tested. Formulation samples placed in PFSs and vials maintained substantial stability over the course of the study (6 months).

### Batch 4 Formulation Study

### Vial Stability Study

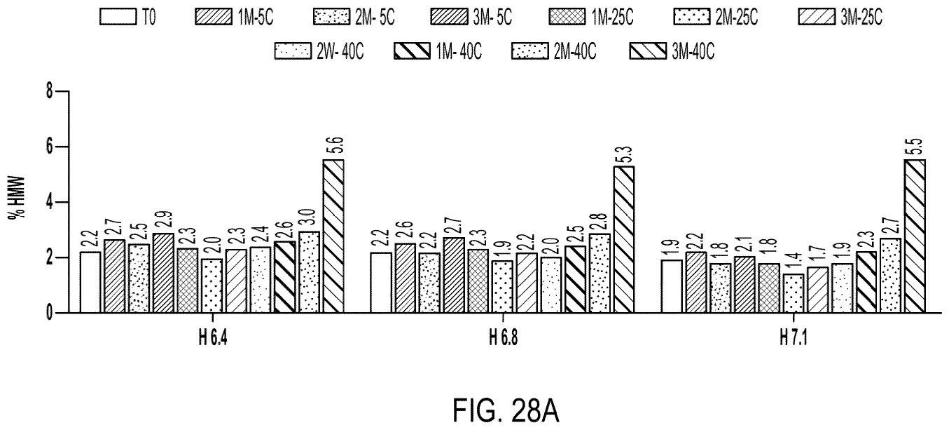

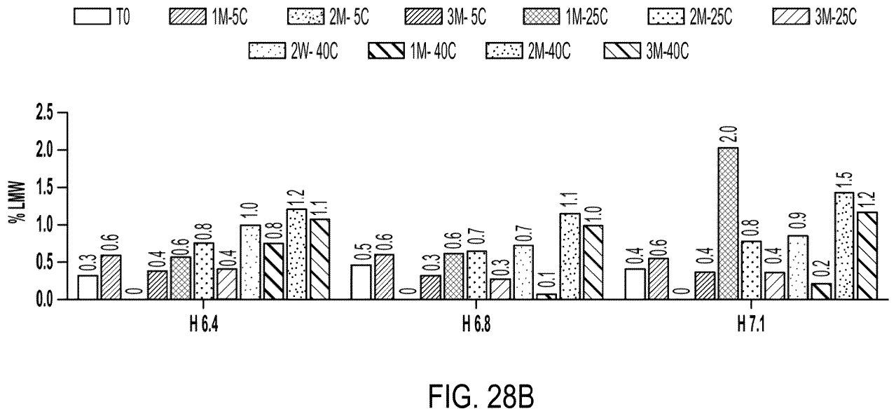

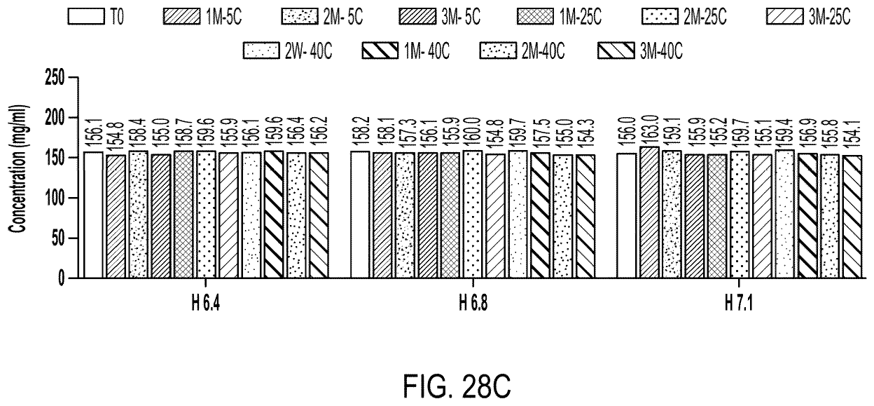

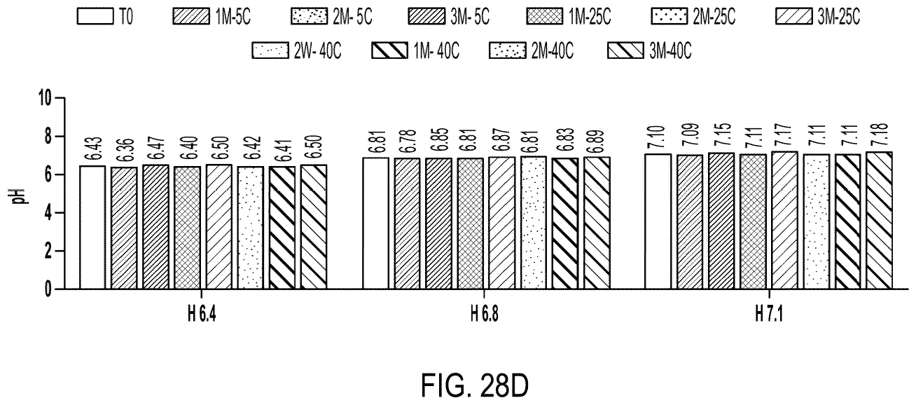

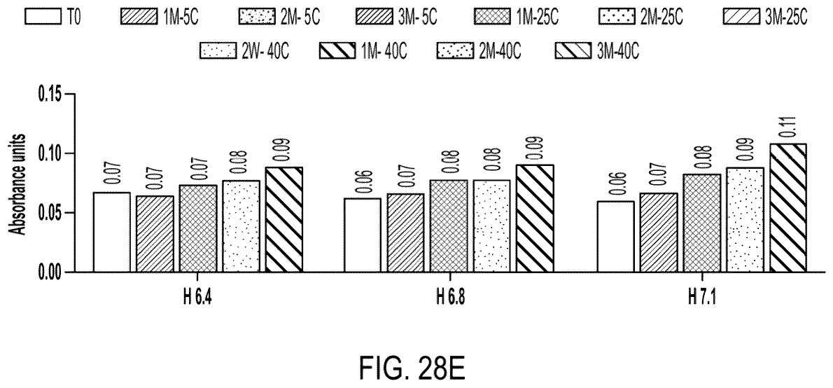

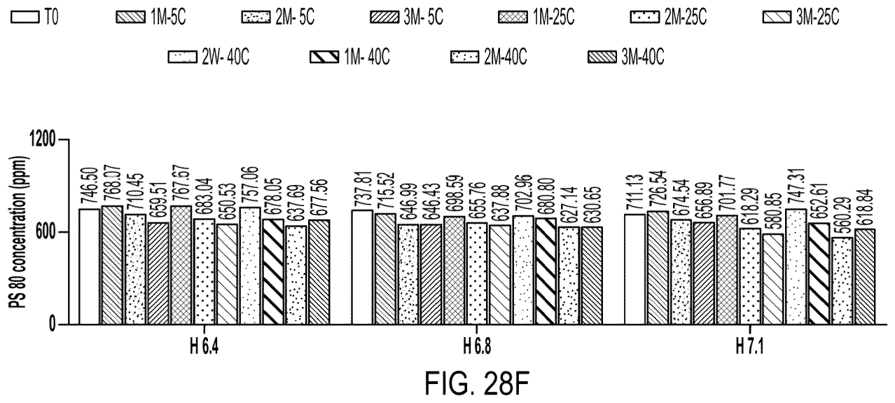

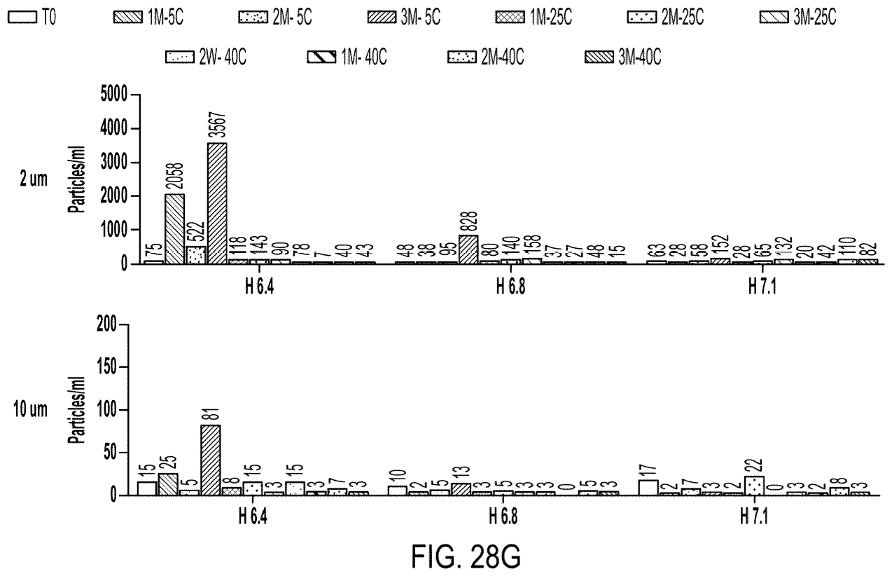

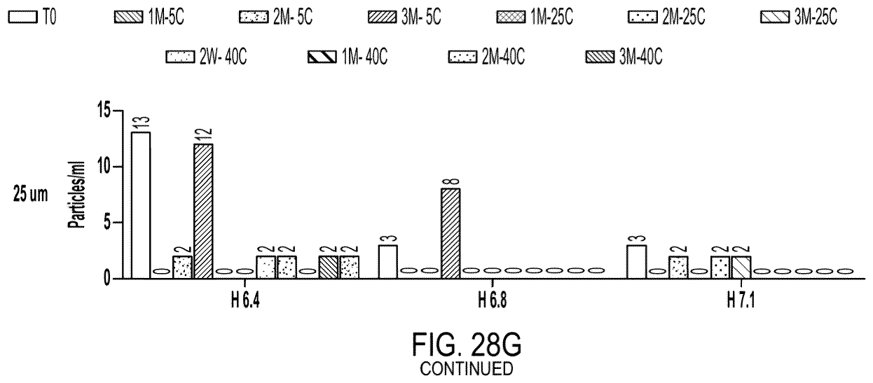

As shown in FIG. 28, an overall increase in HMW species formation over time and at 40° C. was observed, though to a lower extent compared to previous batches. No significant change in level of HMW species formation was observed at 5° C. or 25° C. between pH 6.4-7.1. Slightly lower HMW species formation at T0 and subsequent timepoints at 5° C. and 25° C. was observed for pH7.1 compared to pH 6.4 and pH 6.8 formulations (FIG. 28A). No distinct differences were observed in levels of LMW species formation between the test pHs at 5° C. and 25° C., though a slight increase over time was observed at 40° C. (FIG. 28B). Formulation pH did not have an observable effect on concentration (FIG. 28C) or pH (FIG. 28D) measured at the end of the storage period. A general increase in turbidity with time and temperature was observed for all test formulations, but no significant differences were observed between different pHs (FIG. 28E). Negligible PS80 degradation was observed in any test formulation under all storage stability conditions (FIG. 28F). Lastly, there was no obvious increase in the level of sub-visible particles upon storage at different temperatures in any of the test formulations. level of formation of sub-visible particles≤2 μm, ≤10 μm and ≤25 μm in size was largely consistent, with a couple of outliers attributed to assay variability (FIG. 28G).

### Peptide Map and Biacore Binding Analysis

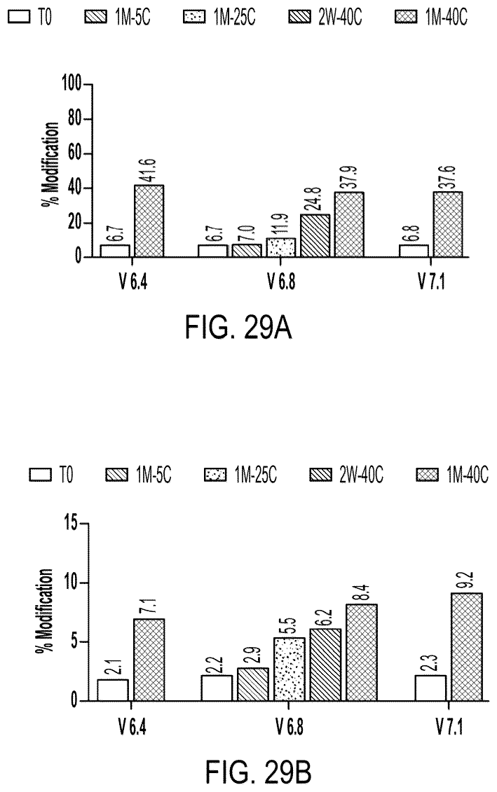

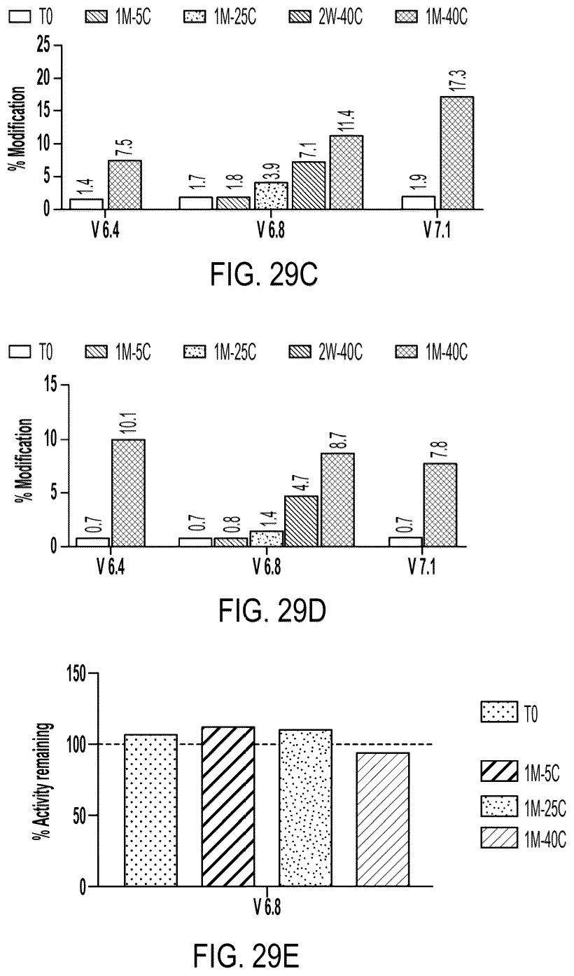

As shown in FIG. 29, a general increase in the level of hotspot modifications as a function of temperature and time was observed in all buffers and pHs tested. Specifically, for D32 isomerization, at 40° C., a modification level of approximately 10% per week was observed in the histidine buffer formulation at pH 6.8 (H6.8). No significant differences in modification levels were observed between different pH values and buffer species (FIG. 29A-D). Biacore analysis indicated that SAR445088 retained relative activity after 1 month storage for samples in histidine buffer at pH 6.8 (FIG. 29E).

### Summary

SAR445088 test formulations having pHs between 6.4 and 7.1 showed no significant differences for all the CQA's tested over the course of the study (3 months).

### Batch 5 Formulation Study

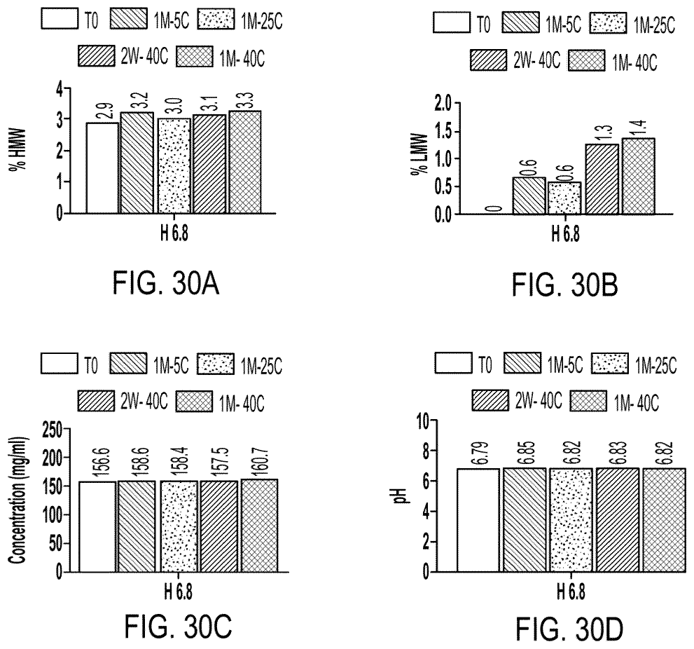

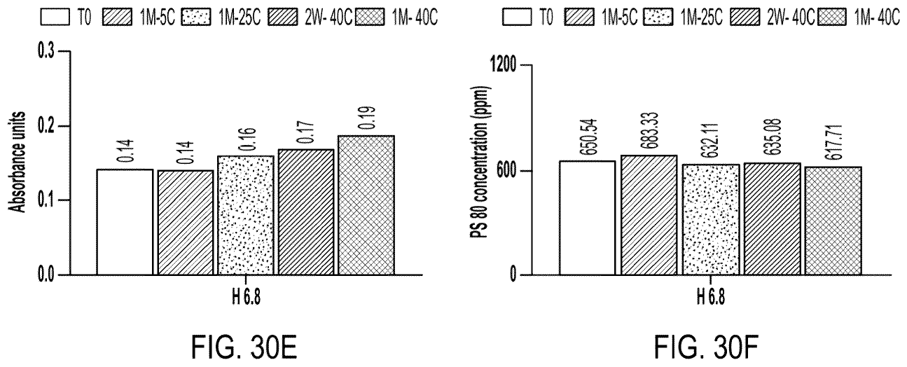

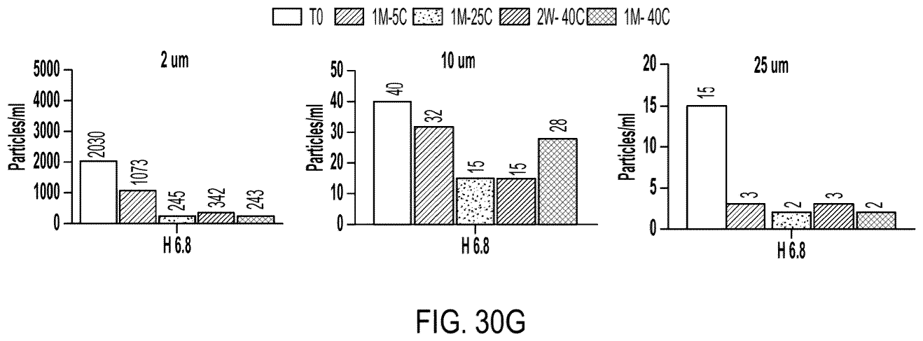

As shown in FIG. 30, a marginal increase in level of HMW species formation over time and at 40° C. was observed, though to a lower extent compared to previous batches. (FIG. 30A). A slight increase in LMW species formation over time was observed at 40° C. (FIG. 30B). Formulation pH did not have an observable effect on concentration (FIG. 30C) or pH (FIG. 30D) measured at the end of the storage period. A general increase in turbidity with time and temperature was observed for all test formulations. (FIG. 30E). Negligible PS80 degradation was observed under any storage stability conditions (FIG. 30F). Lastly, there was no obvious increase in the level of sub-visible particles upon storage at different temperatures. Level of sub-visible particles≤2 μm, ≤10 μm and ≤25 μm in size were largely consistent (FIG. 30G).

### Example 7. Robustness Studies

SAR445088 formulations using different batches of SAR445088 were subjected to various physicochemical stresses as described in Table 5.

TABLE 5
Study conditions for robustness studies.

| STUDY | METHODOLOGY |
|---|---|
| Wrist action shaking | Samples in vials were put on a wrist action shaker |
| (vials) | for 1 h, 3 h and 6 h |
| Wrist action shaking | Samples in PFS were put on a wrist action shaker |
| (PFS) | for 1 h, 3 h and 6 h |
| Freeze-thaw cycling | Samples were subjected for up to five freeze-thaw |
| | cycles between −80° C. and room temperature |
| Photostability | Samples were kept on a shelf at room temperature |
| | and ambient light for 1 day, 3 days, 7 days and 14 |
| | days. A light-protected sample (covered in |
| | aluminum foil) was used as a control. |

### Wrist Action Shaking (Vials)

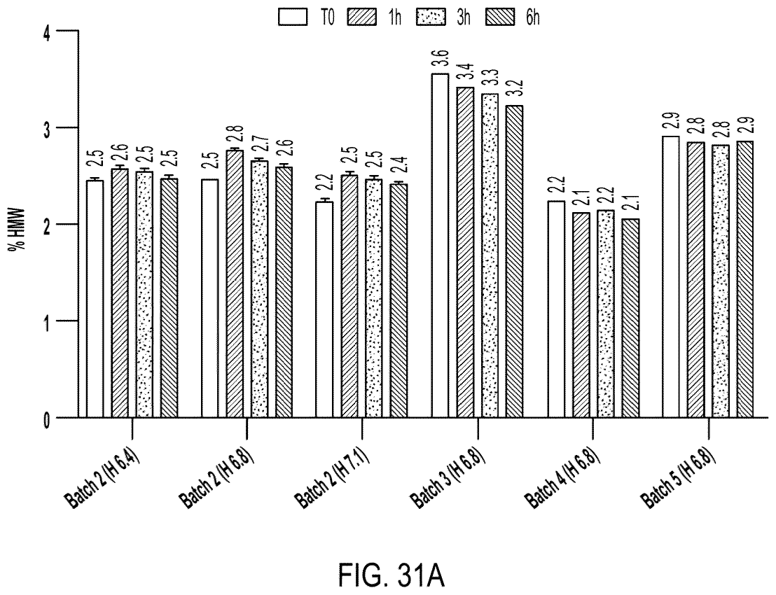

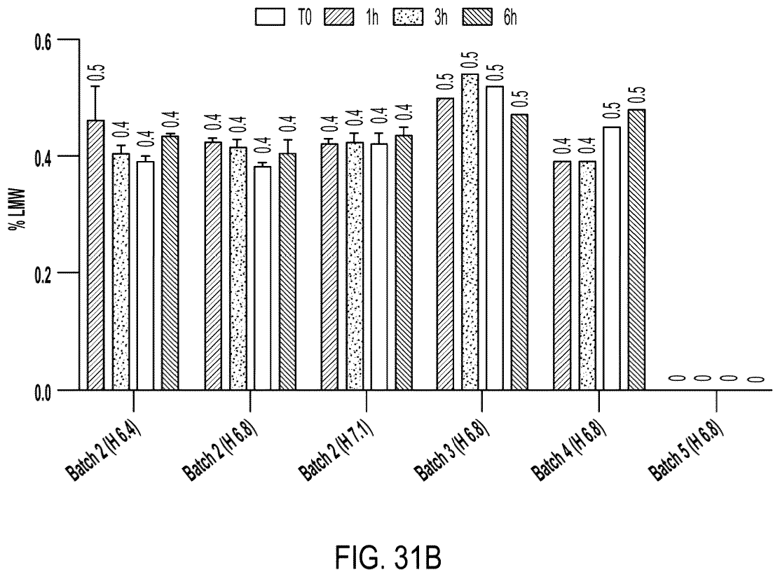

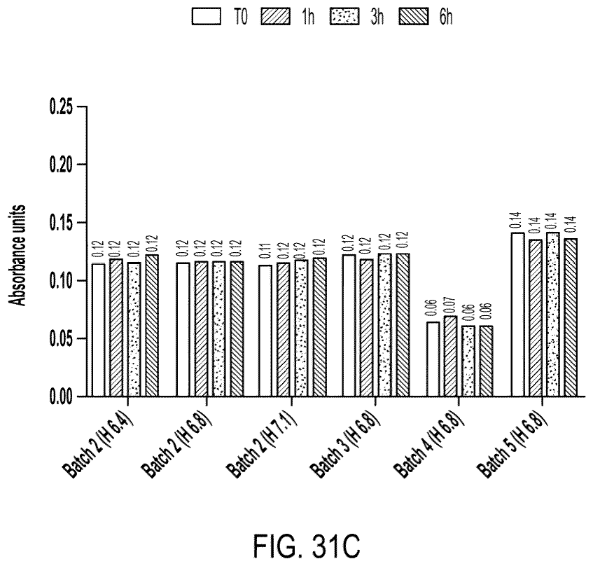

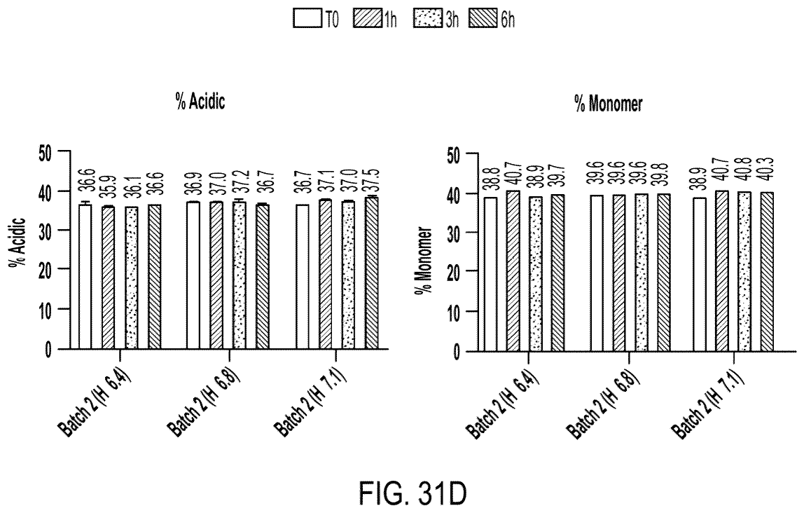

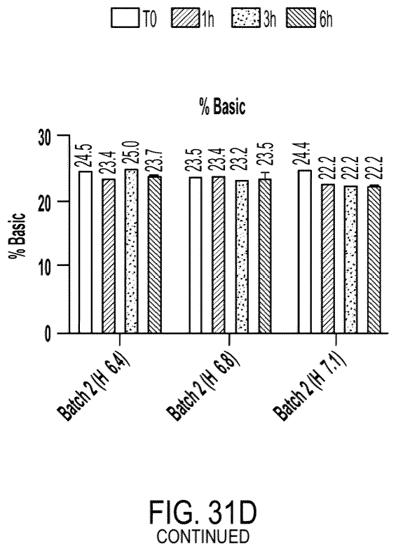

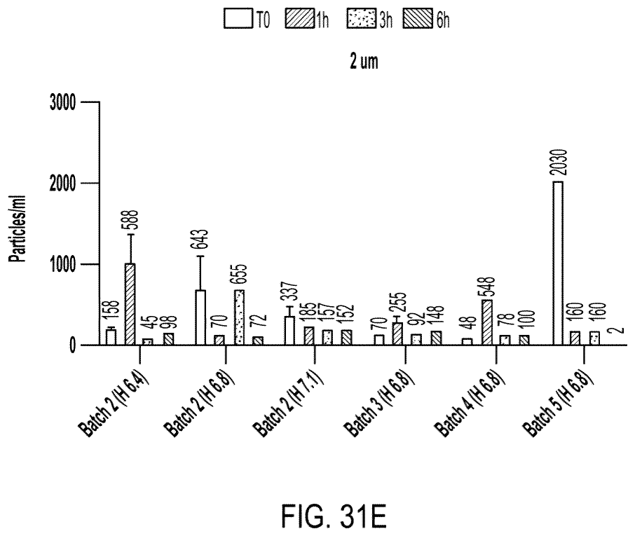

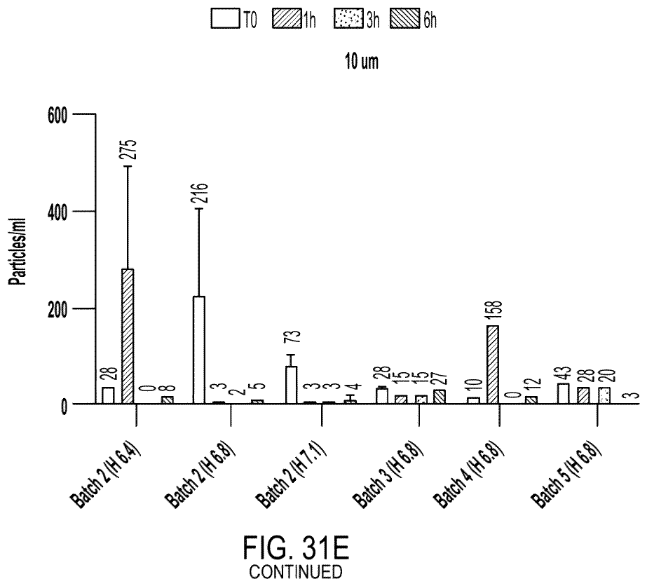

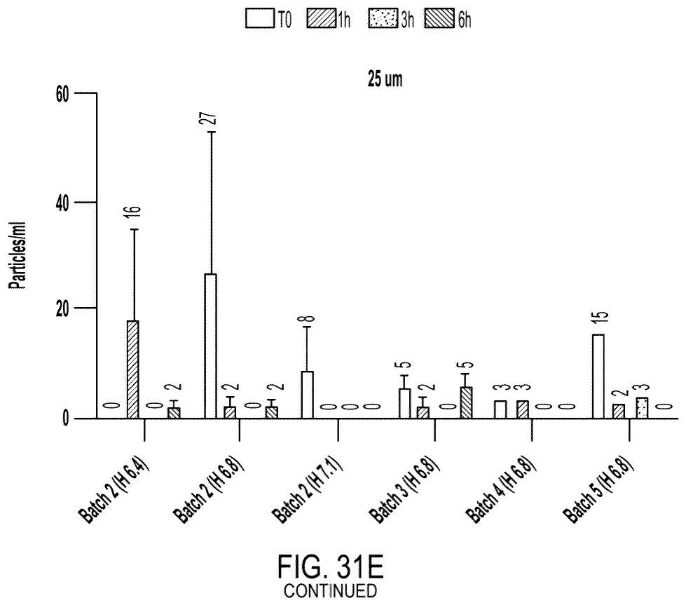

As shown in FIG. 31, the starting HMW species values for different batches varied, but wrist action shaking in vials had no significant impact on level of HMW species formation (FIG. 31A). Wrist action shaking had no observable impact on LMW species formation (FIG. 31B), turbidity (FIG. 31C) or charged variant formation (FIG. 31D). Sub-visible particle analysis indicated that, while the starting sub-visible particles differed between batches, there was no increasing trend in formation of ≤2 μm, ≤10 μm and ≤25 μm sub-visible particles upon wrist action shaking in vials (FIG. 31E).

### Wrist Action Shaking (PFS)

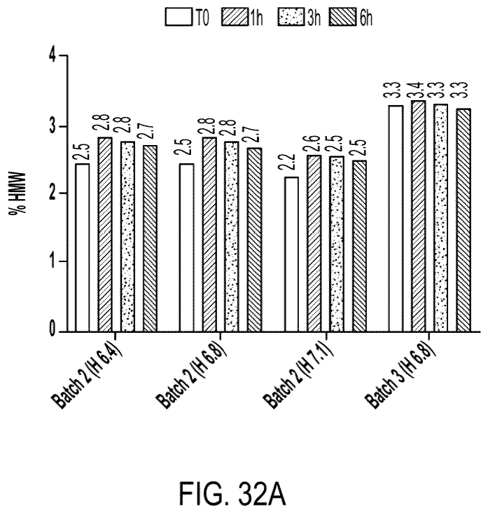

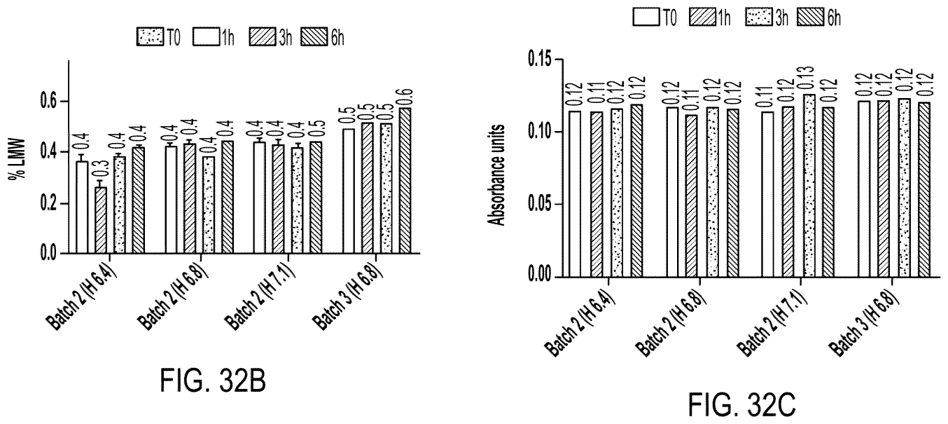

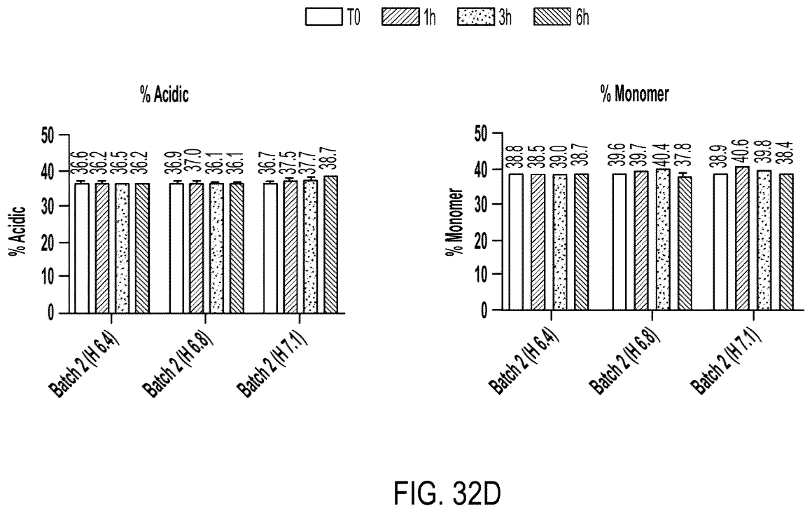

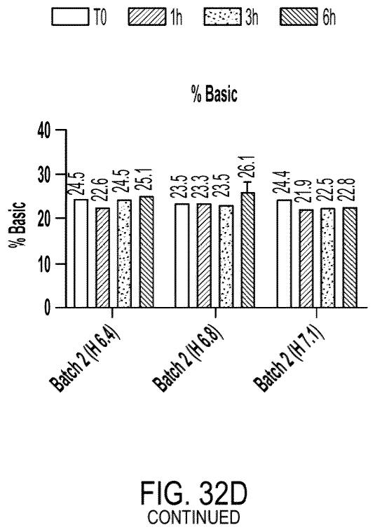

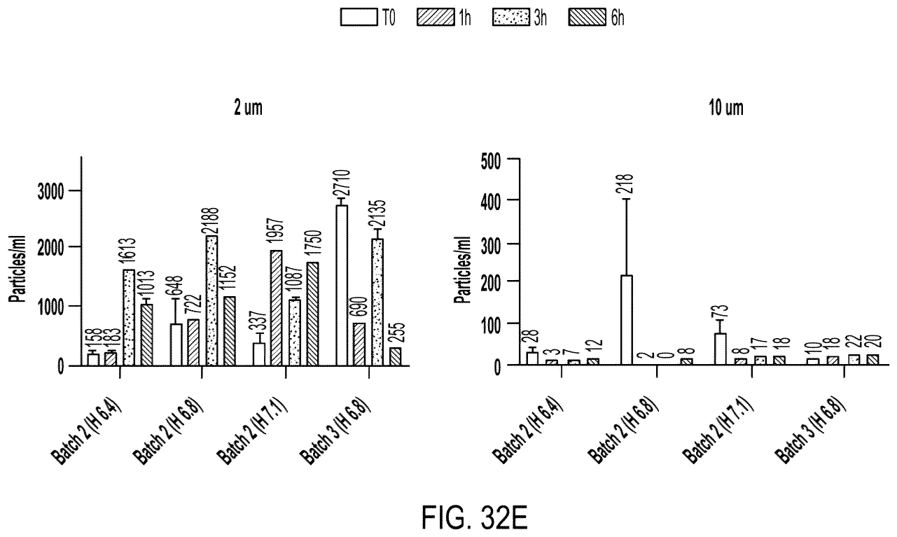

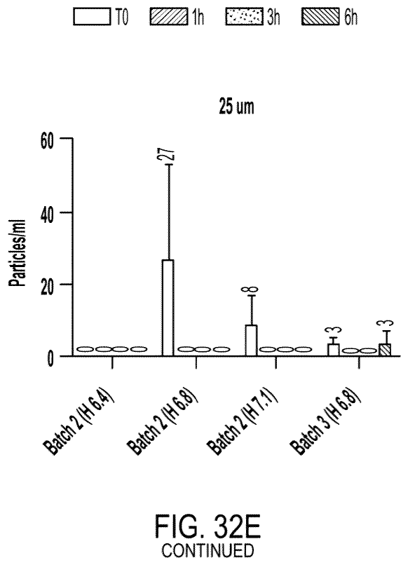

As shown in FIG. 32, the starting HMW species values for different batches varied, but wrist action shaking in PFSs had no significant impact on HMW species formation (FIG. 32A). Wrist action shaking had no observable impact on LMW species formation (FIG. 32B), turbidity (FIG. 32C) or charged variant formation (FIG. 32D). Sub-visible particle analysis indicated that, while the starting sub-visible particles differed between batches and was greater as compared to vials, there was no increasing trend in formation of ≤2 μm, ≤10 μm and ≤25 μm sub-visible particles upon wrist action shaking in PFSs (FIG. 32E).

### Freeze-Thaw Cycling

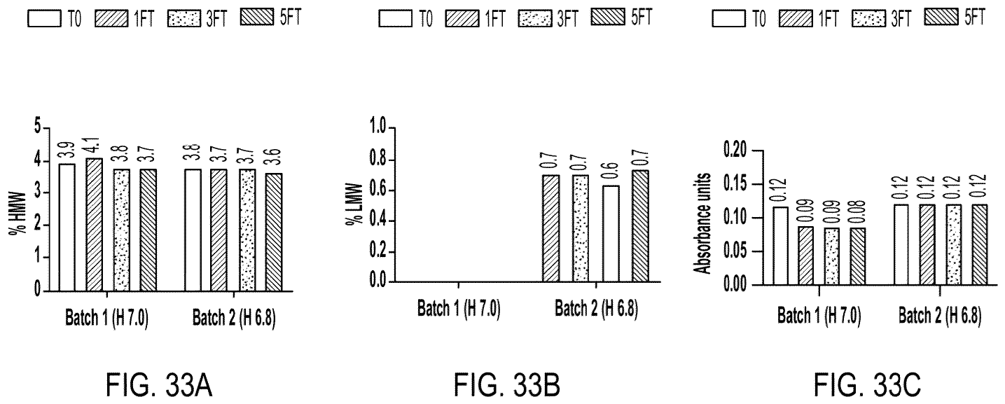

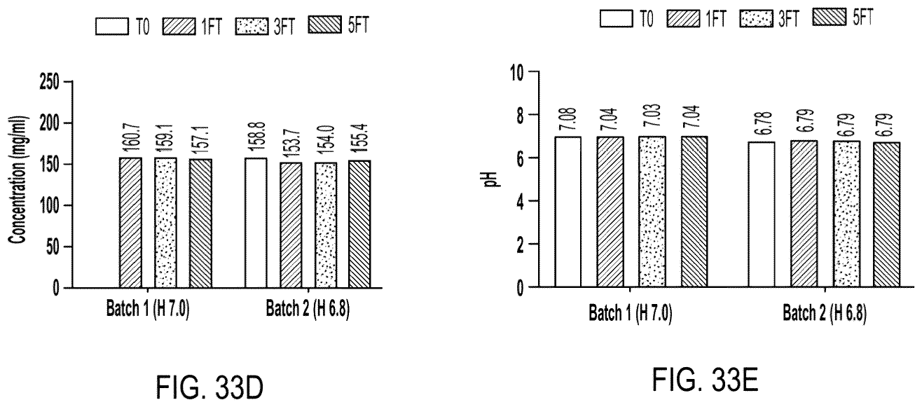

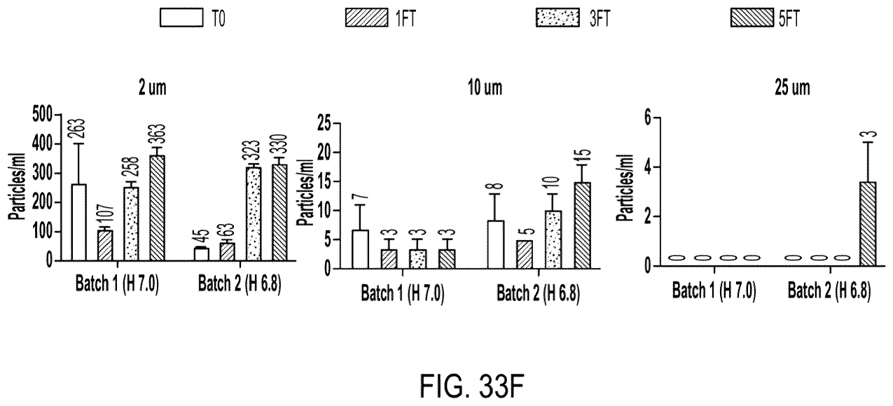

As shown in FIG. 33, the starting HMW species values for different batches varied, but freeze-thawing had no significant impact on HMW species formation (FIG. 33A). Freeze-thawing had no observable impact on LMW species formation (FIG. 33B), turbidity (FIG. 33C), concentration (FIG. 33D) or pH of the formulation measured at the end of the storage period (FIG. 33E). Sub-visible particle analysis indicated a slightly increasing trend in formation of ≤2 μm, ≤10 μm and ≤25 μm particles upon repeat freeze-thaw cycling, however, the particle counts per container remained well within USP specifications (<6000 10 μm particles per small volume container and <600 25 μm particles per small volume container) (FIG. 33F).

### Photostability Study

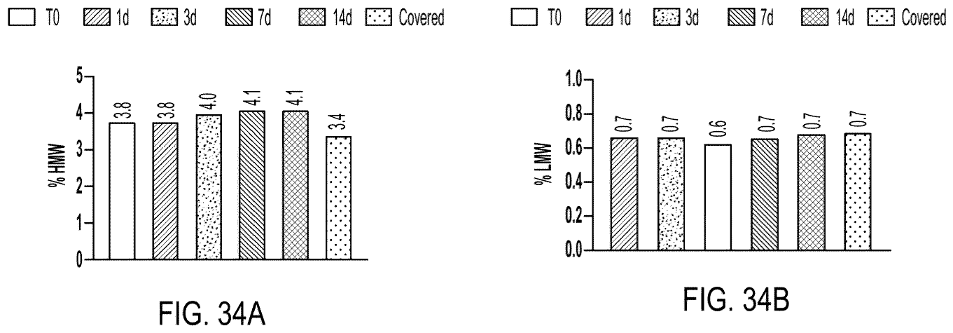

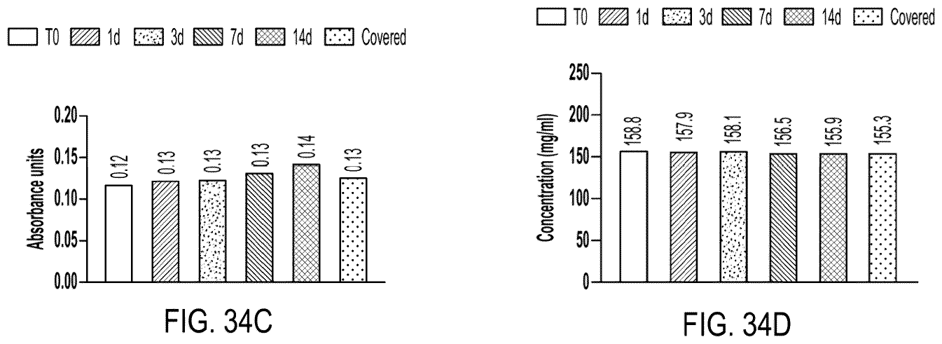

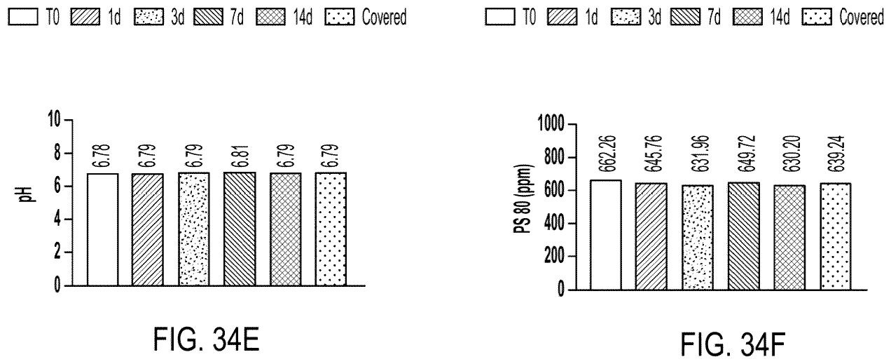

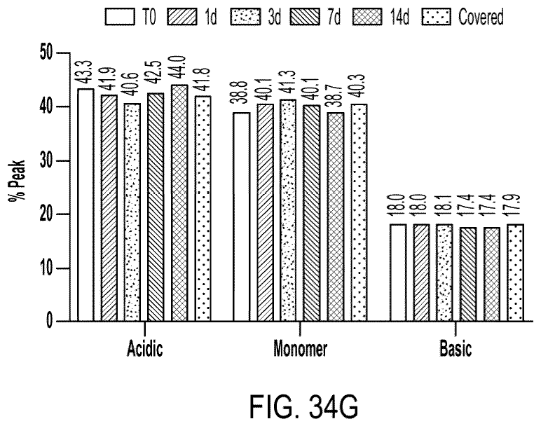

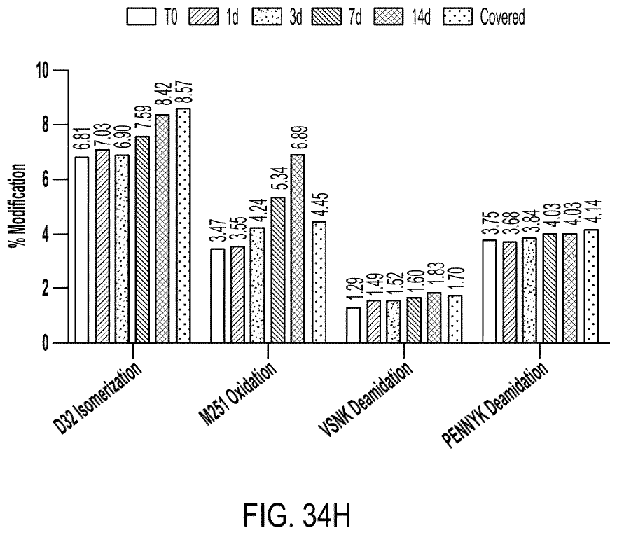

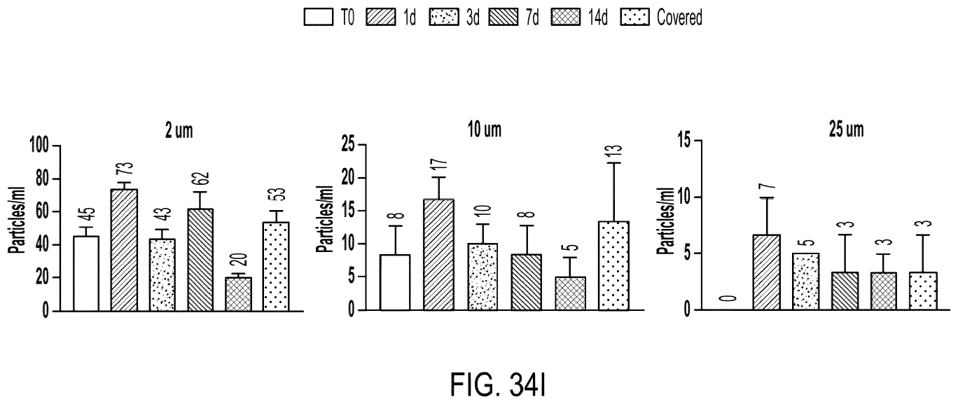

As shown in FIG. 34, a slight increase in HMW species formation was observed upon increased exposure to ambient temperature and light compared to the control sample covered in aluminum foil (FIG. 34A). Exposure to light had no observable impact on LMW species formation (FIG. 34B), turbidity (FIG. 34C), concentration (FIG. 34D) or pH of the formulation (FIG. 34E) measured at the end of the storage period. No PS80 degradation was observed in any sample (FIG. 34F). No significant changes in charged variant formation were observed on exposure to ambient light for up to 14 days (FIG. 34G). Total peptide map analysis indicated that there was an overall increase in D32 isomerization, VSNK deamidation and PENNYK deamidation, including in the covered control sample, indicating that these modifications are not a direct consequence of exposure to light. M251 oxidation, however, increased on increasing exposure to light compared to the covered control. This was an expected effect (FIG. 34H). Analysis of sub-visible particle formation indicated a negligible effect of exposure to ambient light for ≤2 μm, ≤10 μm and ≤25 μm sub-visible particles (FIG. 34I).

### Summary

SAR445088 formulations demonstrated robustness against the physicochemical stresses of wrist action shaking in vials and PFS, freeze-thaw cycling and exposure to ambient light for 14 days. The robustness was observed across different batches and between pH values of 6.4 to 7.1.

### Example 8. Exemplary Liquid Formulation

Formulation characterization studies described herein identified an appropriate concentration of PS 80, identified an optimal type and concentration of chelator, determined an appropriate buffer and pH value that provided DP stability bracketing an optimal pH and that facilitated DP robustness against physicochemical stresses, and evaluated the effect of addition of 3% sucrose, in addition to managing D32 isomerization.

Specifically, a higher pH of 6.8 and inclusion of arginine hydrochloride in formulations reduced the rate of D32 isomerization in LC CDR1 of SAR445088. Such optimized formulations provided resistance to isomerization when tested across different SAR445088 batches under refrigerated, ambient and accelerated storage conditions.

An optimal PS80 concentration was determined by evaluating resistance of formulations containing different PS80 concentrations to mechanical stresses, a simulated clinical dilution and short-term exposure to light. These tests identified 600 ppm (0.06%) PS80 as an optimal surfactant concentration to protect SAR445088 from shear, IV dilution and light exposure effects.

Due to batch-to-batch variability in metal ion content of various drug substance batches and inability of 10 μM EDTA-containing formulations to mitigate PS80 degradation across different batches, a level of 10 μM DTPA was selected as an optimal chelator for mitigating PS80 degradation risk. DTPA was more effective at preventing PS80 degradation (oxidation), reducing HMW species formation and avoiding discoloration of the product solution, which was observed in formulations with 10 μM EDTA or when no chelator was used in the formulation (see FIGS. 8A, 8B, 8D, 9, 10A, 10B, and 10C).

Head-to-head comparison of SAR445088 formulations in histidine and phosphate buffer showed that histidine buffer at pH 6.8 had an optimal ability to reduce aggregation propensity under accelerated stability testing conditions.

Stability studies indicated that addition of sucrose did not negatively impact protein stability, and provided resistance against aggregation that can potentially happen during repeat freeze-thaw cycling.

Different PFSs were compared and demonstrated similar product profiles. DP stability studies in vials and PFS were performed between pH values of 6.4 to 7.1 under refrigerated, ambient, accelerated and frozen conditions. The PFS showed an acceptable product profile in this pH range across different batches, i.e. overall control of HMW species propensity, mitigation of PS80 oxidation and control of molecule-specific D32 isomerization, in each case.

An exemplary SAR445088 liquid formulation shown by the analysis described herein to be optimal for stabilizing the 150 mg/mL liquid DP as well as the 150 mg/mL frozen FDS is 10 mM Histidine, 150 mM Arginine HCl, 3% (w/v) sucrose, 10 μM DTPA, 0.06% (w/v) PS80, at pH 6.8±0.3.

### Example 9. Components of the Drug Product

An SAR445088 DP may be composed of SAR445088, L-histidine, L-histidine hydrochloride monohydrate, L-arginine hydrochloride, sucrose, DTPA and polysorbate 80. These excipients are known to be well tolerated following parenteral administration and are water soluble. Exemplary DP composition and container closure details are summarized in Table 6 below.

TABLE 6
Components of exemplary SAR445088 DP.

| Item | Content/vial | | Details | Contents/PFS | | Details |
|---|---|---|---|---|---|---|
| SAR445088 | 1200 | mg | — | 300 | mg | — |
| L-Histidine | 11.52 | mg | — | 2.88 | mg | — |
| L-Histidine | 1.2 | mg | | 0.30 | mg | |
| hydrochloride | | | | | | |
| monohydrate | | | | | | |
| L-Arginine | 252.8 | mg | | 63.2 | mg | |
| hydrochloride | | | | | | |
| Sucrose | 240 | mg | — | 60 | mg | — |
| DTPA | 0.00003 | mg | | 0.000008 | mg | |
| Polysorbate 80 | 4.8 | mg | — | 1.2 | mg | — |

| | | | | |
|---|---|---|---|---|
| Container | glass vial | 10R Schott | Glass syringe | 2.25 mL OMPI |
| | | glass type I, | | syringes |
| | | topline quality | | |
| Closure | Elastomeric | West 20 mm | Elastomeric stopper | 1-3 mL syringe |
| | stopper with | flurotec coated | | Novapure |
| | aluminum cap | chlorobutyl. | | rubber stopper |
| | | Flanged | | |
| | | aluminum cap | | |
| | | with flip-off | | |
| | | cap | | |

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

1. A composition comprising:
(a) a humanized antibody that specifically binds complement component C1s, wherein the antibody comprises a light chain (LC) complementarity determining region (CDR) 1 comprising the amino acid sequence of SEQ ID NO: 1, an LC CDR2 comprising the amino acid sequence of SEQ ID NO: 2, an LC CDR3 comprising the amino acid sequence of SEQ ID NO: 3 and a heavy chain (HC) CDR1 comprising the amino acid sequence of SEQ ID NO: 4, an HC CDR2 comprising the amino acid sequence of SEQ ID NO: 5, and an HC CDR3 comprising the amino acid sequence of SEQ ID NO: 6; and
(b) about 50 mM to about 200 mM arginine or a salt thereof.

2. The composition of claim 1, wherein the composition comprises about 100 mM to about 200 mM or about 100 mM to about 150 mM of arginine or a salt thereof.

3. The composition of claim 1, wherein the arginine salt is arginine hydrochloride, arginine citrate, arginine oxalate, arginine phosphate, arginine succinate, or arginine tartrate, optionally wherein the composition comprises about 150 mM arginine hydrochloride.

4. (canceled)

5. The composition of claim 1, wherein the composition comprises a buffer at a concentration of about 1 mM to about 50 mM, about 5 mM to about 25 mM, or about 10 mM to about 20 mM.

6. The composition of claim 5, wherein the buffer is histidine, acetate, citrate, oxalate, phosphate, succinate, or tartrate, optionally wherein the composition comprises about 10 mM histidine.

7. (canceled)

8. The composition of claim 1, wherein the composition further comprises a stabilizer at a concentration of about 1% to about 8% (w/v) or about 1% to about 5% (w/v).

9. The composition of claim 8, wherein the stabilizer is sucrose, sorbitol, or trehalose, optionally wherein the composition comprises about 3% (w/v) sucrose.

10. (canceled)

11. The composition of claim 1, wherein the composition further comprises a chelator, optionally wherein the chelator is at a concentration of about 1 μM to about 50 μM or about 5 μM to about 25 μM or about 10 μM to about 25 μM.

12. The composition of claim 11, wherein the chelator is diethylenetriamine pentaacetate (DTPA), ethylenediaminetetraacetic acid (EDTA), or methionine, optionally wherein the composition comprises about 10 μM DTPA.

13. (canceled)

14. The composition of claim 1, wherein the composition further comprises a surfactant at a concentration of about 0.01% to about 0.1% (w/v) or about 0.03% to about 0.06% (w/v).

15. The composition of claim 14, wherein the surfactant is polysorbate 80 (PS80) or poloxamer 188 (P188), optionally wherein the composition comprises about 0.06% (w/v) PS80.

16. (canceled)

17. The composition of claim 1, wherein the composition has a pH of about 6 to about 7.5, about 6 to about 7, about 6.5 to about 7.5, or about 6.5 to about 7.1, optionally wherein the composition has a pH of about 6.8.

18. (canceled)

19. The composition of claim 1, wherein the composition comprises about 50 mg/mL to about 250 mg/mL, or about 100 mg/mL to about 200 mg/mL of the anti-C1s antibody, optionally wherein the composition comprises about 150 mg/mL of the anti-C1s antibody.

20. (canceled)

21. The composition of claim 1, wherein the composition comprises:
(a) about 50 mg/mL to about 250 mg/mL of the antibody;
(b) about 50 mM to about 200 mM arginine HCl;
(c) about 1 mM to about 50 mM histidine;
(d) about 1% to about 8% (w/v) sucrose;
(e) about 1 μM to about 50 μM diethylenetriamine pentaacetate (DTPA); and
(f) about 0.01% to about 0.1% (w/v) polysorbate 80 (PS80), and
wherein the composition has a pH of about 6 to about 7.5.

22. The composition of claim 21, wherein the composition comprises:
(a) about 150 mg/mL of the antibody;
(b) about 150 mM L-arginine HC1;
(c) about 10 mM histidine;
(d) about 3% (w/v) sucrose;
(e) about 10 μM DTPA; and
(f) about 0.06% (w/v) PS80, and
wherein the composition has a pH of about 6.5 to about 7.1, or about 6.8.

23. The composition of claim 1, wherein the antibody comprises a light chain variable region (VL) comprising the amino acid sequence of SEQ ID NO: 7 and a heavy chain variable region (VH) comprising the amino acid sequence of SEQ ID NO: 8.

24. The composition of claim 1, wherein the antibody is a Fab fragment, a F(ab′)₂ fragment, a scFv, or a Fv.

25. The composition of claim 1, wherein the antibody comprises a heavy chain constant region of the isotype IgG4, optionally wherein the IgG4 constant region comprises a proline, a glutamic acid, a leucine, and a serine substitutions at amino acid residues 108, 115, 308, and 314, respectively, relative to the IgG4 constant region sequence of SEQ ID NO: 11.

26. (canceled)

27. The composition of claim 1, wherein the antibody comprises a light chain comprising the amino acid sequence of SEQ ID NO: 9 and a heavy chain comprising the amino acid sequence of SEQ ID NO: 10.

28. The composition of claim 1, wherein the antibody has a lower isomerization rate at D32 of the light chain CDR1 as compared to the antibody in a corresponding formulation without arginine or a salt thereof, optionally wherein the D32 isomerization is determined by total peptide map analysis.

29. The composition of claim 1, wherein the antibody:
(i) has an isomerization rate that is at least about 2-3% lower per week at 40° C., at least about 1-3% lower per month at 25° C. or at least about 0.4-0.6% lower per month at 5° C., as compared to the antibody in a corresponding formulation without arginine or a salt thereof;
(ii) has an isomerization rate after 12 weeks of storage at 25° C. that is about 10% lower as compared to the antibody in a corresponding formulation without arginine or a salt thereof; and/or
(iii) has an isomerization rate of less than 7.5% after 12 weeks of storage at 5° C., less than 20% after 12 weeks of storage at 25° C., or less than 35% after 12 weeks of storage at 40° C.

30. (canceled)

31. The composition of claim 1, wherein the composition is in liquid, lyophilized, or reconstituted lyophilized form.

32. (canceled)

33. A container containing the composition of claim 1.

34-35. (canceled)

36. A kit or an article of manufacture, comprising the container of claim 33.

37. A pharmaceutical unit dosage form suitable for parenteral administration to a human, comprising a composition according to claim 1 in a container.

38. A method comprising administering to a human the composition according to claim 1.

39. A method of reducing the level of a complement component cleavage product in a human, the method comprising administering to the human the composition according to claim 1.

40. A method of inhibiting C1s-mediated cleavage of a complement component in a human, the method comprising administering to the human the composition of claim 1.

41. (canceled)

42. A method of treating a complement-mediated disease in a human in need thereof, comprising administering to the human the composition of claim 1.

43-44. (canceled)

45. A drug delivery device comprising a primary container containing a composition of claim 1, wherein the drug delivery device is a sleeve-triggered auto-injector with manual needle insertion.
